SEQUENCE LISTING (32 sequences)

SEQ ID NO: 1 - DNA, 9591 bp, Solanum lycopersicum
  misc_feature 1..9591: note = genomic MeR1
  source 1..9591: mol_type = genomic DNA
tttcgcacttcactgaaacattaaattgggcaacattactctgttatactaataatacagattttaggtgccttgatatcaaacatagagtaaattcaattattttcgtgttcatcacattcctcatctatttttctcttacttattttcctcctttttagcaggagagtatcgaggcatggtgatgcatttgcagtctcagttttgattacctgttccaataaagaaacttgttacaagtatgttttgttgattcaaacatgccaagtttgaaaacgaagttttatattaatgtttttgcgttttctttcagtgctgaataatttgtctaatctgtagttgatgtttggatatctatgtattgttcgattgacacgagttgtttatactatcatttgttgtttatgaatatgtaaaacatatttgatttatgtttgcaacgtcaacgtgaacattcaattagtctaatcaaagtctagatctaaaattgattaatttaagttcataatttattttgaattattatctaaaaccgaagactttctactcgaaaatcgaaatcatagctgacagtctccataaatatatttgcaattgaataacataataagcatttatcagttaattttatggagtttctggatttacgtgatattcaaacatctcccgcgcatattaattatgtgaagtaatgaagtgtgtaacataagtcaaaaggtgcataaataaagagtcaagggtaataagactttagtttactttaggtgtgtctctgaaatttcgattataatctacgatatattgtgtcttgtcccaacatttaaataaaatgaacaaataattttaattaccaaatggatccatagtctgtttagttgactaatggaaagatttataggaaggtgagattgttttatccctgattagaggttttggttcgagttttgtgtatacgaaaaaattgtgttgaaagcgtcacttatgaataggccctataatgtatgattcgaatttagtctgagttctaatatgaattttgaacacattgaaaaattatatcctttgttaaatttcatggttttaacatcttggaagtaaagaaaaaggtaaaatattttttttttctatttcatttttttacatgtaatatttgaattcaaaggctgagagagtaataccccccttttatttttatttgtggattacatatatacataagaactagaaaaatgtttatattttcgacttgacaatgatagaatgtaaatgttggatatatagaataatttgttggagcttctaccattttttgcttaccgtcttaaggttgaaggcgtagatgtttattatccgaataattcctttttgtcgataatcaagcatcaatattagattagtagccgaccctcttctcttacctctctaaacaatttttcaatatttccattttggttggtaaaaatattagagaacattttctctatagaaatatcatgtgtttattcagaattgttattgactccatatatttttaataatccaaaacctataaatttaatagtttacattcacaattattgcttgttgctgttcttttaccttgtgtttctaatttttaccgttattaaatatgcatgtgattagttgaaatattaaatctctagaaattgaaggctggctcgaggttagagattaaatggaaaaatgaaagtcaacgaagcatggtagctgtcaaatacgctccaactcaaagcattactgtcatgatggagttcctattgatccatcgtgaaaaattgaacgatatgttggcacatgtcggaatacttaaaatatcgaattaatataattgaatttaactttgaaaattaatcaaattaactttcgaaaagcgctacctgacaattaaaaatacaaggagtgagcagtgaacttaaactaatctatttcagatctctaggctttgataaactaattgacagttcaacatttgcagatctaacttcaacgtttgcttcttgtagatctgcaacaaggcaaactaccatttgagagtaaagaaaaaggtataatataatgtttttctagttttcattttacatgtatttttgtgattcacttgatttgaatgaaacatacattacaagaactagaaaacgaatttgtatttttgacttagataattataaaatgttagatgattttgtcataatttgtagtattcactcttgtttttagtaaatatacctctttcttaaatgtgattgatttacagattatatattttagaatttttgcttattaaaagaaaaattaattagtggcggaaatttgaggtgaatttgtgaagaatggaagaagttggaaattcgagagtttgttcatgtgttttttaaggtagaagaaacaaaaaaaatccatttgatctaaaattccaattgaaatgtattacaagttgtttggggtaaaaaataatgacatgaatgtgtttttctcaaatgataatgacatagatgagccaaagttttaactgatgacataaatgaacctttctttttctacaaagttcgataacatatttgagccaaaatttagaatttataacagctgacattcttttaactctttcttcttgctgttacttttacttgtgtttctaattcttttatccttattaaatgtggatgtgattagtttgaaaaatcaaattttgtagatattgaaggctggttggatggaaaaaggagaaaaatcattggtatgtctattgtctaccaatactgtagtttctagcgttaagagccaatattttcaactgaattttgctgatgcatatgtttttgatttagcagctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacgggtatgggaaatatacagggaaggagaggaatttaaaaagtttagaacttgaaaggttgggaacttgaaaagtactctaatgctttattgaaaaagataatagtccctcatcggtaatggaaatgaaaataggagagtttaaataaataaacactcctattaaattatttttttttacaaataaatttctgttcatcttatttactggttctaaaaatctcagttacttcgtgtttctgaattatttattataaccagtaacttctgattttcttaacacagattggcaacagagataactaagttcgtttaatttatataatttgcatttgtgtagtgtgttcattgaagttgttagtagagttcattcttctttctgcacacactaccacaaaaacaactttttagaggcaataaacaaatcatccactgatttgactagtcgtaacattatgagaaaattcaatggatttgttttgtattgcaggtggagaatgtacttgtgaagaactataatacttagatcctgaatgtttgatttgtgcttatacatagttggtggagtgattgcactgattctcgtggttatgaatcgcagtggttgatgaagttggatttctcatggaaaaatcacaacaatgaagaacgaaacaacgcaacgcctccatctgattactgcttccaataaacaaacttgttactggttttctcattgctggttctggacgtattttgatctaatgtgtcgtgtgttttctttcctgtttttttgttagttatgaatcttatttcgtctgtaatctctcatataagattctagtttgtttgtttttttaattggaaggtttcaatgccaaattgtgaatcaatataggtcccatttggcatgaaaaaggcaactaactatcactattgatatggacagttaattagataactactgtgccaacaagcattgtggtggagaggcagtactctctgatccttaatcagaggtctcgcgttcgagccctaccagatatagaatcgcctatgtatgtcttactctcaatgtgatactatttttcaatattaaatatgtatcaaatatagaatataattatcgaaactatcttaaatccttgcaattaggtcctaaactattattattattattatataataaactaataaagtaacatcaaacttgccattttttattcttgtcacctaattacatgattgaataatgttttttcccgactattattgatccactagcaaatatttatatggacagtaggaaattatatagaaagactttgctttaataattcaaaatcatagtaaactttgcttaaactaatctatttcacatctagtctttaattgcttcttgcttttgctcactcaattttgtcagtgaaagttttgtaaatctgcaaactaccatttaggatgaagtaactcttttaacagctgacattcttttaactctttcttcttgctgttacttttacttgtgtttctaattcttttatccttattacaagtgcatgtgattagtttgaaaaatcaaattttgtagatattgaaggctggttggatggaaaagggagaaaaatcattggtatgtctattgtctaccaatactgtagtttctagcgttaagagccaatattttcaactgaattttgctgatgaatatgtttttgatttagcagctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgccttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagttaatagagtaacacaattctgttttgatctttggacaagaaagtataaagactttgacaatgagtatgccttctctgaatgttcttccaagatcacttctctactcatcgacataatccctcttgagttggaggttctacacatttctacacattgttaagcaaatcctaaaagcatctccaaggattcttcaaaagcatctcattcatctccaaggacgcatggtagccgtcagttactctacaactcaaagcattaatgtcatgatggagttcctattgatctttctcactgatataccaaagcgctttatccatcgtggcaaattgaactctatgttggcacatgtcggactacttacaaggaagatatctattctcatggaagagagctctaatatgaatgaagcagacttttcagctacatacttgttgcaagaaatcgaacgaatgaagagagatatcaaacagattattttgaaagcgccagagtcatcccaactttgccttcctatggatgatggtttcctcttcatgaatcttctactccgacatttaaatgatttactcacttccaattcttattcagtttctctgataaaaaaagaaattgagatggtgaaacaatgccttgaattcctaacaacatctttcaggcaaacattggatgagagcactagtggagttgttaaagattgttggatgcgtgctttggatg

SEQ ID NO: 2 - DNA, 3840 bp, Solanum lycopersicum
  misc_feature 1..3840: note = CDS MeR1 (isoform 1)
  source 1..3840: mol_type = genomic DNA
atggaaaaaggagaaaaatcattgctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacgggtatgggaaatatacagggaaggagaggaatttaa

SEQ ID NO: 3 - DNA, 3843 bp, Solanum lycopersicum
  misc_feature 1..3843: note = CDS MeR1 (isoform 2)
  source 1..3843: mol_type = genomic DNA
atggaaaaaggagaaaaatcattgcagctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacgggtatgggaaatatacagggaaggagaggaatttaa

SEQ ID NO: 4 - DNA, 3834 bp, Solanum lycopersicum
  misc_feature 1..3834: note = CDS MeR1 (isoform 3)
  source 1..3834: mol_type = genomic DNA
atggaaaaaggagaaaaatcattgctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacgggtggagaatgtacttgtgaagaactataa

SEQ ID NO: 5 - DNA, 3837 bp, Solanum lycopersicum
  misc_feature 1..3837: note = CDS MeR1 (isoform 4)
  source 1..3837: mol_type = genomic DNA
atggaaaaaggagaaaaatcattgcagctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacgggtggagaatgtacttgtgaagaactataa

SEQ ID NO: 6 - DNA, 1899 bp, Solanum lycopersicum
  misc_feature 1..1899: note = CDS MeR1 (isoform 5)
  source 1..1899: mol_type = genomic DNA
atggaaaaaggagaaaaatcattgctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtggagaatgtacttgtga

SEQ ID NO: 7 - DNA, 1902 bp, Solanum lycopersicum
  misc_feature 1..1902: note = CSD MeR1 (isoform 6)
  source 1..1902: mol_type = genomic DNA
atggaaaaaggagaaaaatcattgcagctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtggagaatgtacttgtga

SEQ ID NO: 8 - DNA, 3849 bp, Solanum lycopersicum
  misc_feature 1..3849: note = CDS MeR1 (isoform 7)
  source 1..3849: mol_type = genomic DNA
atggaaaaaggagaaaaatcattgctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacggtggttgatgaagttggatttctcatggaaaaatcacaacaatga

SEQ ID NO: 9 - DNA, 3852 bp, Solanum lycopersicum
  misc_feature 1..3852: note = CDS MeR1 (isoform 8)
  source 1..3852: mol_type = genomic DNA
atggaaaaaggagaaaaatcattgcagctactatttgaggaacaaaaaacaagaatcacgaatcttattgatgatttcttgattcgcttgaagcaaataaagaatgaattcattgcttcaaaattggatgcctttgaaaatctaagaatggaactgagattcctaagaacatttgtcctgtttgggaattcgatgaatttggatgacttttatgagaggatgtcactgagtataagcaaattcgatcaatctacggagtacgtagataaaataatcctagagaaatacaacatggaatgtcttgcccctctgctgcttgaagagataagaaattatttgagtttgaagaatgattatgtagccacaactacagagataaaattgtttgaatacctcatcagaaacctccatgatctaccaaagtattgttctgatttgcttctaccactcatgagtgaatacaagattcttcggcaagtatgcacacatctcagagatttctatcagttggaatgcaacaaaacaacaaaaacagaatttctctatactcggtatcaagtgacagccgatagagtaacacaattctgttttgatctttggacaggaatgtataaatactctgacaatgagtatgccttctctgaatgttcttccaagatcacttctctactcattgacataatccctcttgagttggaggttctatacatttctacttctaagctcatcaaagagtcaacgtcaaaagaactagaaggatttgttaagcaaatcttaaaagcatctccaaggattcttcaacattatctcattcatctccaaggacgcatggccggtgtagaagccgtcaatttcgctccaactcgaagcattagtgtcatgatggagttcctattgatctttctcaccgatatgccaaagcggtttatccatcgtgaaaaattgaacgatatgttggcacatgtcagaatacttacaaggaaaatatctactctggtgagtaaactgttggaggagatctctgaggataatatcaatgaagcggacttttcagctccggactttttgcaagaaattgaacaaatgaagggagatatcagacacatctttttaaaagctcctgagtcatctcaacttcggtttcctatggatgatggtttcctcttcatgaatcttctactcagacatttaaatgatttactcatttccaatgcttattcagtttttctcataaaaaaagaaattgggatggtgaaagaaagccttgaattcctaatatcatctttcaggaaagtcaggcaaacattggatgagagtactagtggagtagttaaaaattgttgggtgcgtgctttggatgtggcatatgaggcagaacatatcattaattccattcttgtcagagataaagctctctcacatctcctcttctcacttccgagtgtcactgataagatcaaacttatcgtggaacaagtcaccaggtttcagctggaggataagaatggggatggcccccttgatgcaaagtcttccttcgagccaactcagtcaacttcatcaccttttgttgaggtaacagtaggtcacgagaaagaagaatcccagatcattgaccagctccttgatgaacatgaatctgagcttgatgtcatttccattgtcggaatgccaggactcggtaaaactactctggccaacaaagtgtataaagatacattagttgctaggcatttccatgtccgtgcttggtgcactgtttcgcaaaagtataacaagtcaaaggtgttgcgggagattcttcagcaagttactggctcgggaggaaaagaaagtgaagatgacctggctgaaaagctacgaagagcactactcgataaaaggtacctaatcgtcttggatgatgtgtgggatattgcaacaggggagatgttaatagcatgttttcctaagggtaagagaggaaatagaatcatcttaactagccgaagtagaaaggtaggtttgaaagttaaatgccgtagtgatcctctcgaccttaaacttttaacatctgaagaaagttgggatttattcgaaaaaagggtatttggagatgaaggaagctgccctgctgaactgtccgaagttggacaccaaatagttgagaaatgtaagggtcttcccttggctattgttttaattgctggagtaattgttagagggaagaaaaaggaaaaggatttttggcttaagatactgcataatctggattcctttatttctaccaacatcaatttggttatgcaattaagttatgaccatttaccatgccacctgaagccgttgctgctttactttgcaacaactcaaaagagccaacaaactccagtctctacattgatgcagttgtggatggccgaagggtttgtggatcatgatagtttagaggaagtaactcaaagttacttggatgctctaatttccagtagcctgataatggtggatcatatcccctccaagagttattggtggacgtctttaatgatcaaggtttgctatgtgcatgatgttgtgcacgatttttgttcagaaaaagccaaaaaggagaagtttctcaagttaatcaattcaggtgatccatttcatgcttcagatttcctacaccatcgtctaaccattcatactgacaacggccaactccacaaaaaatgtgttttattcaattctaataagtgcttagctgttagtaagcatgtcatatctttgaaagtgagtggtccactagatgagttcaggtatatctgtcacacaagacactttggacttgttagagtgttgcaactggatgacatcattctggaagattctttaatggaagaaatagggtccctatttcatttgaggttcttaagcattgagactgctgtaggtgtaatagctatcccagtgtcgtggttgaacctccagaatctggaaacgctgttgatttatacaacttattccaccatggtattactgcccagaatattgcaactgtcaaagctgaaacatgtgaaaattaaggaatgttctttctttgaagagaaagaggatatccaacgtagaatattggaagctgggaattcttcaaacttgacaactctatccggagttgttatctcatattctgaaggcatgagtgatgatgctctggagaagttcccaattcttcagcaccttgattgcatcatcatggaatcgcagaatgctcctacacacgactattggtttctcaagcttgatgtacttaataaactcgaatcattcgtagcaagatacaagcgcaatggacatccctcgttaaaccgacaaccgtatggatttcacttccctacaagcttgaaagagttacggttgactggttttttcctgagacctgatttgttgtcagtaatcgcagcgttgcctgagcttgagattatggagttttccggctgttatttcgtggatactaaatgggacgcaagtgaggacatctatctaagtcttaagactttgattttgcgagatgtccatttatcagaatggcaagttgaggggggaacttttcccaagcttgagaaattaatactaaaattttgttccacacttggggagatcccttgtgcatttatggatgtagaaactttaaagtccattgatttaagttttgttgggcgtaagcttcgagattcagccattgagattaagaaaaatgtagcagatttcacaggagaggacagagttgatgtccacacatcacatttgtttgcaaccaacgtgaaagaacagattatgagaatgacggtggttgatgaagttggatttctcatggaaaaatcacaacaatga

SEQ ID NO: 10 - protein, 1279 aa, Solanum lycopersicum
  REGION 1..1279: note = protein MeR1 (isoform 1)
MEKGEKSLLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRYLIVLDDVWDIATGEMLIACFPKGKRGNRIILTSRSRKVGLKVKCRSDPLDLKLLTSEESWDLFEKRVFGDEGSCPAELSEVGHQIVEKCKGLPLAIVLIAGVIVRGKKKEKDFWLKILHNLDSFISTNINLVMQLSYDHLPCHLKPLLLYFATTQKSQQTPVSTLMQLWMAEGFVDHDSLEEVTQSYLDALISSSLIMVDHIPSKSYWWTSLMIKVCYVHDVVHDFCSEKAKKEKFLKLINSGDPFHASDFLHHRLTIHTDNGQLHKKCVLFNSNKCLAVSKHVISLKVSGPLDEFRYICHTRHFGLVRVLQLDDIILEDSLMEEIGSLFHLRFLSIETAVGVIAIPVSWLNLQNLETLLIYTTYSTMVLLPRILQLSKLKHVKIKECSFFEEKEDIQRRILEAGNSSNLTTLSGVVISYSEGMSDDALEKFPILQHLDCIIMESQNAPTHDYWFLKLDVLNKLESFVARYKRNGHPSLNRQPYGFHFPTSLKELRLTGFFLRPDLLSVIAALPELEIMEFSGCYFVDTKWDASEDIYLSLKTLILRDVHLSEWQVEGGTFPKLEKLILKFCSTLGEIPCAFMDVETLKSIDLSFVGRKLRDSAIEIKKNVADFTGEDRVDVHTSHLFATNVKEQIMRMTGMGNIQGRRGI

SEQ ID NO: 11 - protein, 1280 aa, Solanum lycopersicum
  REGION 1..1280: note = protein MeR1 (isoform 2)
MEKGEKSLQLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRYLIVLDDVWDIATGEMLIACFPKGKRGNRIILTSRSRKVGLKVKCRSDPLDLKLLTSEESWDLFEKRVFGDEGSCPAELSEVGHQIVEKCKGLPLAIVLIAGVIVRGKKKEKDFWLKILHNLDSFISTNINLVMQLSYDHLPCHLKPLLLYFATTQKSQQTPVSTLMQLWMAEGFVDHDSLEEVTQSYLDALISSSLIMVDHIPSKSYWWTSLMIKVCYVHDVVHDFCSEKAKKEKFLKLINSGDPFHASDFLHHRLTIHTDNGQLHKKCVLFNSNKCLAVSKHVISLKVSGPLDEFRYICHTRHFGLVRVLQLDDIILEDSLMEEIGSLFHLRFLSIETAVGVIAIPVSWLNLQNLETLLIYTTYSTMVLLPRILQLSKLKHVKIKECSFFEEKEDIQRRILEAGNSSNLTTLSGVVISYSEGMSDDALEKFPILQHLDCIIMESQNAPTHDYWFLKLDVLNKLESFVARYKRNGHPSLNRQPYGFHFPTSLKELRLTGFFLRPDLLSVIAALPELEIMEFSGCYFVDTKWDASEDIYLSLKTLILRDVHLSEWQVEGGTFPKLEKLILKFCSTLGEIPCAFMDVETLKSIDLSFVGRKLRDSAIEIKKNVADFTGEDRVDVHTSHLFATNVKEQIMRMTGMGNIQGRRGI

SEQ ID NO: 12 - protein, 1277 aa, Solanum lycopersicum
  REGION 1..1277: note = protein MeR1 (isoform 3)
MEKGEKSLLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRYLIVLDDVWDIATGEMLIACFPKGKRGNRIILTSRSRKVGLKVKCRSDPLDLKLLTSEESWDLFEKRVFGDEGSCPAELSEVGHQIVEKCKGLPLAIVLIAGVIVRGKKKEKDFWLKILHNLDSFISTNINLVMQLSYDHLPCHLKPLLLYFATTQKSQQTPVSTLMQLWMAEGFVDHDSLEEVTQSYLDALISSSLIMVDHIPSKSYWWTSLMIKVCYVHDVVHDFCSEKAKKEKFLKLINSGDPFHASDFLHHRLTIHTDNGQLHKKCVLFNSNKCLAVSKHVISLKVSGPLDEFRYICHTRHFGLVRVLQLDDIILEDSLMEEIGSLFHLRFLSIETAVGVIAIPVSWLNLQNLETLLIYTTYSTMVLLPRILQLSKLKHVKIKECSFFEEKEDIQRRILEAGNSSNLTTLSGVVISYSEGMSDDALEKFPILQHLDCIIMESQNAPTHDYWFLKLDVLNKLESFVARYKRNGHPSLNRQPYGFHFPTSLKELRLTGFFLRPDLLSVIAALPELEIMEFSGCYFVDTKWDASEDIYLSLKTLILRDVHLSEWQVEGGTFPKLEKLILKFCSTLGEIPCAFMDVETLKSIDLSFVGRKLRDSAIEIKKNVADFTGEDRVDVHTSHLFATNVKEQIMRMTGGECTCEEL

SEQ ID NO: 13 - protein, 1278 aa, Solanum lycopersicum
  REGION 1..1278: note = protein MeR1 (isoform 4)
MEKGEKSLQLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRYLIVLDDVWDIATGEMLIACFPKGKRGNRIILTSRSRKVGLKVKCRSDPLDLKLLTSEESWDLFEKRVFGDEGSCPAELSEVGHQIVEKCKGLPLAIVLIAGVIVRGKKKEKDFWLKILHNLDSFISTNINLVMQLSYDHLPCHLKPLLLYFATTQKSQQTPVSTLMQLWMAEGFVDHDSLEEVTQSYLDALISSSLIMVDHIPSKSYWWTSLMIKVCYVHDVVHDFCSEKAKKEKFLKLINSGDPFHASDFLHHRLTIHTDNGQLHKKCVLFNSNKCLAVSKHVISLKVSGPLDEFRYICHTRHFGLVRVLQLDDIILEDSLMEEIGSLFHLRFLSIETAVGVIAIPVSWLNLQNLETLLIYTTYSTMVLLPRILQLSKLKHVKIKECSFFEEKEDIQRRILEAGNSSNLTTLSGVVISYSEGMSDDALEKFPILQHLDCIIMESQNAPTHDYWFLKLDVLNKLESFVARYKRNGHPSLNRQPYGFHFPTSLKELRLTGFFLRPDLLSVIAALPELEIMEFSGCYFVDTKWDASEDIYLSLKTLILRDVHLSEWQVEGGTFPKLEKLILKFCSTLGEIPCAFMDVETLKSIDLSFVGRKLRDSAIEIKKNVADFTGEDRVDVHTSHLFATNVKEQIMRMTGGECTCEEL

SEQ ID NO: 14 - protein, 632 aa, Solanum lycopersicum
  REGION 1..632: note = protein MeR1 (isoform 5)
MEKGEKSLLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRWRMYL

SEQ ID NO: 15 - protein, 633 aa, Solanum lycopersicum
  REGION 1..633: note = protein MeR1 (isoform 6)
MEKGEKSLQLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRWRMYL

SEQ ID NO: 16 - protein, 1282 aa, Solanum lycopersicum
  REGION 1..1282: note = protein MeR1 (isoform 7)
MEKGEKSLLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRYLIVLDDVWDIATGEMLIACFPKGKRGNRIILTSRSRKVGLKVKCRSDPLDLKLLTSEESWDLFEKRVFGDEGSCPAELSEVGHQIVEKCKGLPLAIVLIAGVIVRGKKKEKDFWLKILHNLDSFISTNINLVMQLSYDHLPCHLKPLLLYFATTQKSQQTPVSTLMQLWMAEGFVDHDSLEEVTQSYLDALISSSLIMVDHIPSKSYWWTSLMIKVCYVHDVVHDFCSEKAKKEKFLKLINSGDPFHASDFLHHRLTIHTDNGQLHKKCVLFNSNKCLAVSKHVISLKVSGPLDEFRYICHTRHFGLVRVLQLDDIILEDSLMEEIGSLFHLRFLSIETAVGVIAIPVSWLNLQNLETLLIYTTYSTMVLLPRILQLSKLKHVKIKECSFFEEKEDIQRRILEAGNSSNLTTLSGVVISYSEGMSDDALEKFPILQHLDCIIMESQNAPTHDYWFLKLDVLNKLESFVARYKRNGHPSLNRQPYGFHFPTSLKELRLTGFFLRPDLLSVIAALPELEIMEFSGCYFVDTKWDASEDIYLSLKTLILRDVHLSEWQVEGGTFPKLEKLILKFCSTLGEIPCAFMDVETLKSIDLSFVGRKLRDSAIEIKKNVADFTGEDRVDVHTSHLFATNVKEQIMRMTVVDEVGFLMEKSQQ

SEQ ID NO: 17 - protein, 1283 aa, Solanum lycopersicum
  REGION 1..1283: note = protein MeR1 (isoform 8)
MEKGEKSLQLLFEEQKTRITNLIDDFLIRLKQIKNEFIASKLDAFENLRMELRFLRTFVLFGNSMNLDDFYERMSLSISKFDQSTEYVDKIILEKYNMECLAPLLLEEIRNYLSLKNDYVATTTEIKLFEYLIRNLHDLPKYCSDLLLPLMSEYKILRQVCTHLRDFYQLECNKTTKTEFLYTRYQVTADRVTQFCFDLWTGMYKYSDNEYAFSECSSKITSLLIDIIPLELEVLYISTSKLIKESTSKELEGFVKQILKASPRILQHYLIHLQGRMAGVEAVNFAPTRSISVMMEFLLIFLTDMPKRFIHREKLNDMLAHVRILTRKISTLVSKLLEEISEDNINEADFSAPDFLQEIEQMKGDIRHIFLKAPESSQLRFPMDDGFLFMNLLLRHLNDLLISNAYSVFLIKKEIGMVKESLEFLISSFRKVRQTLDESTSGVVKNCWVRALDVAYEAEHIINSILVRDKALSHLLFSLPSVTDKIKLIVEQVTRFQLEDKNGDGPLDAKSSFEPTQSTSSPFVEVTVGHEKEESQIIDQLLDEHESELDVISIVGMPGLGKTTLANKVYKDTLVARHFHVRAWCTVSQKYNKSKVLREILQQVTGSGGKESEDDLAEKLRRALLDKRYLIVLDDVWDIATGEMLIACFPKGKRGNRIILTSRSRKVGLKVKCRSDPLDLKLLTSEESWDLFEKRVFGDEGSCPAELSEVGHQIVEKCKGLPLAIVLIAGVIVRGKKKEKDFWLKILHNLDSFISTNINLVMQLSYDHLPCHLKPLLLYFATTQKSQQTPVSTLMQLWMAEGFVDHDSLEEVTQSYLDALISSSLIMVDHIPSKSYWWTSLMIKVCYVHDVVHDFCSEKAKKEKFLKLINSGDPFHASDFLHHRLTIHTDNGQLHKKCVLFNSNKCLAVSKHVISLKVSGPLDEFRYICHTRHFGLVRVLQLDDIILEDSLMEEIGSLFHLRFLSIETAVGVIAIPVSWLNLQNLETLLIYTTYSTMVLLPRILQLSKLKHVKIKECSFFEEKEDIQRRILEAGNSSNLTTLSGVVISYSEGMSDDALEKFPILQHLDCIIMESQNAPTHDYWFLKLDVLNKLESFVARYKRNGHPSLNRQPYGFHFPTSLKELRLTGFFLRPDLLSVIAALPELEIMEFSGCYFVDTKWDASEDIYLSLKTLILRDVHLSEWQVEGGTFPKLEKLILKFCSTLGEIPCAFMDVETLKSIDLSFVGRKLRDSAIEIKKNVADFTGEDRVDVHTSHLFATNVKEQIMRMTVVDEVGFLMEKSQQ

SEQ ID NO: 18 - DNA, 8391 bp, Solanum lycopersicum
  misc_feature 1..8391: note = Genomic NRC6-2a
  source 1..8391: mol_type = genomic DNA
attcaattttaaaaaattattcagtaaaattattaaattttattttgtatcgttaaaattactcaattttgatatatttaaatcagtgaaattattaaactaaatttatcattaaaataataaaataattttttaaatatataattacaaataaaataataaataaatacttaaaattactactttattatatattgtaagataaaataataaaagataattttatcaatttcatttggtacactgcttcattattgcaatgacgactgtcgattttgctccggtagtttaatcaatttgtagtggtttaatcaattttttgtttgtgtgaatcccttatttaatttttttttaaaaaaaataaataaagtgaggtttcatgtatcaatggtagtttggacaattttgagtagtttgataaatttattttttgttttgaatttttattattttataatgaaatagtaatttcgagtcatgtactatgtagtttaatcataaacatagattgagtgattttaatgatataaaaacctaaattgagtattttactggtacaaaatgaagtttagttgctagatatatttcaaaagttgagtgaccttttgagaaattaactcctctgtcagatctcctaatttgataaattttcaaaacttcataatataacattttccttttttataatgattacaaactatatcaaacgacgagttccccctgtcacttatacttttaattcacaaattcctcttatcagtgtgaccacatccccacaaccatacttgttacgatccaaaaatgagcgtgatgacactcgtcttattcacatagattagtcagcctaaaactcaattattatgaagaagagaggaaataaactataaagtggaatcagattcatgagttgtgtttgagtctgaattattgaatattaaagtctgatcaattaaagtcgagagtaattagtctgtataatttgttctatgtttattactgagttgtaataatttctatattttgtaaccaaaaacggatttgactttttgaagagagtttgagagaattgtaacaagaaatgtgtttgacttatcgcagagtagagttaagagagagttagagagaattgtaataagaaatgagtttgacttcttagtgagtagagagttgagagagagtttgagagaattataacaagaagtttgacttcttggagaatagagtttccgattataattaatcgttgaagagtaaagatactagacttagtccctttgatgattgaattgttattgaagttaataaaattgtgatgtgattttttccttccttgagggaggaaggcttccacggtaaattaagtgttgtttaaattctgttctaaacgactataggaaccaggttcctagaaataattgttggcgcatagtatctttcaaggaaaaatgcagaatgccacgtaagctcgaaaagggtagaaaattatctataaaataagttcgggtggaaggataataagagttgtataagtgtgtctctgaaatttgagacatagattaggaggtacttatgtattttcccttcttccagttatttgtttcacctcttccataatgtcaaatccttaattatatatgtctacctctgtacgtcatattatagatagagaaacaatttaaatgtgtctttataatcaatatcactgaaagctctagcgacgttaaatacaacaatattatttttttattttgctaccttgaataataattctcaattgtttacaagctttccaacaacacccatttatttcaaccaatgtgaacctctttccaacaacacttaatttgttttctagtaatttttcaatcaacatgaacttaattattcaaatgttagctgtcattggattattaaattgttaatgttgtaaggtgatggtgtattatattaagaattaacaaataatacttaaagacttggttaatctaattcagagtcaacttgaagttatttcattccacatgtttataaacacacaactatggcaataacaacatagcaattgtcatctctaaattactactactattcttcttctccatcaatttaatcctcagaaatggatccgtttacggcggctgcattaaccactgcggtgacagccacggtgagccttctggtggagaacttgtcgcatcttataagttataattggaagttgtatacaggattgaagaaatcatgcgaagatttgtatgatgaagtgaagcgattaaatgcatttttagtcgataacgcgaatcagagaagtaatagtacgcaatgggatgtattagtcgataaaattcgacgtacagtatataaagcagaggatgttgttgataaattattgattcagggcaagttagaccaagagagtaatatagctaaaaagatgtttcacaaaacttacaaaaacaggaattttactgaggaaatcaatgagatacttgtagaggtgaggaaaatccttgaggaaaatcaacatctgtttgaggcaaacccgacgattgatcatcatcagcctgaaaaagttgtccaggaggaacaggtacgtttacacgaaatttcgattttaatgtgatttattgaaatatgaaatgaactatgatttgctcgttcagtaaaactgtataagagttggagaaatagtacttcctctgttttaatatgcattatttgtttgaacttagttaaagtttaatctaaattattcagatctaggacaaggcattaggcataagtaaggcaaagggtagaagtacctttctagattatgactcaaattccgtagaattttactttagttaaggtgtgtctctgaaattcagacaattaaaggtcaccaccaaccattattacatgcacaaatcactatcgataattgccaccattggtcaacattatatatcgctgacctccatctatcattaactgtcagtatgattccactattaattatcaaatggtcatcaacaacaaccacaattaatcactaaggccaatcactatactgtccacaaccactatcatctgtcaacactatctaaaatcgatacacacaatcactatcattagtcattaccaccaccgattgtcacattttcttgtagtgaatgatggggattgaaaatatgaagaataaaatagacatgcataggataaggggacatatttttcatagttgaatgggacattgaaaattaataactttcacatgatcataactcatttggtacctgatggaataatttcttgttatgatgatacaatcaagacaagtagccttaataacttgtttaatttgtccatcttacagggttcgtcattggaaaatcacgaagtggttggatttgatgaagaagcaacgaaagtgatcaatcgtctggttgaaggagcagagtgtctagatgttatcccggttgtaggaatgccgggacttggtaaaaccacactggcaagaaaaatctttaatgatcctaagatttcgcgagaatttttcagctacatttgggttttcatcggacaatcaacgtgtgtaaaaagggatatcctttttaatattctgaaagggttcacaaattcatttgatgaattcaaaaacagaaatgaggcaagcataactgatgaaatacgtaagcgtgtggctaatggaggtaaatgtctcattgtcttggatgatgtgtgggatacaaatgttgtagatattgtcaagacagttttccctgataacaaaaaagcccacaggatcatgatgaccactcgacacgaagacattgctagatctgtcaataaatatccccacaatctgaaatttctggatggagacgaaagtttccagctgctagaaaagagagcttttggcgttagccgttgtcctgttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggatttgatatccccgcttggaaattgattcgactctggattgctgagggactcataatgtccaagttgtcgggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacagaactcaattacttgtcgtcgtgtctctattcaatcatctgttcctcaaaacttcatctcaaaaaagacagttgaagaacatgttaggtcattgttatgtttttcctcaaaacaaaaacaagttgacttttctaatatcgacgtcaagctcatccctaccgcatttccacttatgagagtcttagacattgaatccgtcaagtttagtattcccagggaattttaccagctattgcacttgaggtatatcgctatgtcaggtgatttcgagcaacttcctaaactctttacttctttctgtaatgcacaaactctcattctaaatacttccaagcccacccttgatataaaagctgacatatggaacatgccacgtttgcgtcatctgcacaccaacaaacctgcaatcttaccaccccctacaagtagtagtagtagtagtacaaattcttgtttgttgcaaactctatctctggttacaccagaaagctgcaagggaaacgttctttcaaaggctcgtaatgtcaagaaaatgagtgttaaaggtaatttgacaccttttcttgaaactagcaagggtgaatttttcagcaattttcaagtgctaaagctcctggaaagtttaacactgctaaatgatgataagagtaataaatctcttcaccttccgtcagcattctccgaatgtttaccaaatttgaagaagttaactctatcaaaaacaaggtttgactggaatcaggcatatagattagggcaggtgaaaaatctccaggtcctaaaactgaaagaaaatgcattcacggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatctttcccatttggataaccttcaagagatgacgttggaaaacacaagcaaagcaagcaaatctgcaagagaaatagaatgcgagaaaaagaagaagcaagctgatgatccagaaactggcaaattcaagctcactattccctactgaagctgatttcaatgccacaaagtgaagttgttataaggttagtccaattctctccctctcgtatcagtaaatatcgaataagatatttttcaataagattgcatctatccctatagtctctccacgcagaattagtaggtagcagtgtgttgtcttgctgtctcgttcatatcaatagtcatagtattgctcctatagtttcttatccttcgatttttcgttactatacgttgtcccttatacctcgatattagtaatgcttgttgtagtttctcttatgttactatctgctgtttttgttattacctgttgtttcttgtatttccgtcatttctttttgggactcctttgaactttctccttgagacaaaggtcaatcggaaacaacctacctctctttctttgagataaggttcctccccgactctattttgtgggactacactgagtatattgttttgggtttcgcacttcactgaaacattaaattgggcaacattactctgttatactaataatacagattttaggtgccttgatatcaaacatagagtaaattcaattattttcgtgttcatcacattcctcatctatttttctcttacttattttcctcctttttagcaggagagtatcgaggcatggtgatgcatttgcagtctcagttttgattacctgttccaataaagaaacttgttacaagtatgttttgttgattcaaacatgccaagtttgaaaacgaagttttatattaatgtttttgcgttttctttcagtgctgaataatttgtctaatctgtagttgatgtttggatatctatgtattgttcgattgacacgagttgtttatactatcatttgttgtttatgaatatgtaaaacatatttgatttatgtttgcaacgtcaacgtgaacattcaattagtctaatcaaagtctagatctaaaattgattaatttaagttcataatttattttgaattattatctaaaaccgaagactttctactcgaaaatcgaaatcatagctgacagtctccataaatatatttgcaattgaataacataataagcatttatcagttaattttatggagtttctggatttacgtgatattcaaacatctcccgcgcatattaattatgtgaagtaatgaagtgtgtaacataagtcaaaaggtgcataaataaagagtcaagggtaataagactttagtttactttaggtgtgtctctgaaatttcgattataatctacgatatattgtgtcttgtcccaacatttaaataaaatgaacaaataattttaattaccaaatggatccatagtctgtttagttgactaatggaaagatttataggaaggtgagattgttttatccctgattagaggttttggttcgagttttgtgtatacgaaaaaattgtgttgaaagcgtcacttatgaataggccctataatgtatgattcgaatttagtctgagttctaatatgaattttgaacacattgaaaaattatatcctttgttaaatttcatggttttaacatcttggaagtaaagaaaaaggtaaaatattttttttttctatttcatttttttacatgtaatatttgaattcaaaggctgagagagtaataccccccttttatttttatttgtggattacatatatacataagaactagaaaaatgtttatattttcgacttgacaatgatagaatgtaaatgttggatatatagaataatttgttggagcttctaccattttttgcttaccgtcttaaggttgaaggcgtagatgtttattatccgaataattcctttttgtcgataatcaagcatcaatattagattagtagccgaccctcttctcttacctctctaaacaatttttcaatatttccattttggttggtaaaaatattagagaacattttctctatagaaatatcatgtgtttattcagaattgttattgactccatatatttttaataatccaaaacctataaatttaatagtttacattcacaattattgcttgttgctgttcttttaccttgtgtttctaatttttaccgttattaaatatgcatgtgattagttgaaatattaaatctctagaaattgaaggctggctcgaggttagagattaaatggaaaaatgaaagtcaacgaagcatggtagctgtcaaatacgctccaactcaaagcattactgtcatgatggagttcctattgatccatcgtgaaaaattgaacgatatgttggcacatgtcggaatacttaaaatatcgaattaatataattgaatttaactttgaaaattaatcaaattaactttcgaaaagcgctacctgacaattaaaaatacaaggagtgagcagtgaacttaaactaatctatttcagatctctaggctttgataaactaattgacagttcaacatttgcagatctaacttcaacgtttgcttcttgtagatctgcaacaaggcaaactaccatttgagagtaaagaaaaaggtataatataatgtttttctagttttcattttacatgtatttttgtgattcacttgatttgaatgaaacatacattacaagaactagaaaacgaatttgtatttttgacttagataattataaaatgttagatgattttgtcataatttgtagtattcactcttgtttttagtaaatatacctctttcttaaatgtgattgatttacagattatatattttagaatttttgcttattaaaagaaaaattaattagtggcggaaatttgaggtgaatttgtgaagaatggaagaagttggaaattc

SEQ ID NO: 19 - DNA, 2670 bp, Solanum lycopersicum
  misc_feature 1..2670: note = CDS NRC6-2a
  source 1..2670: mol_type = genomic DNA
atggatccgtttacggcggctgcattaaccactgcggtgacagccacggtgagccttctggtggagaacttgtcgcatcttataagttataattggaagttgtatacaggattgaagaaatcatgcgaagatttgtatgatgaagtgaagcgattaaatgcatttttagtcgataacgcgaatcagagaagtaatagtacgcaatgggatgtattagtcgataaaattcgacgtacagtatataaagcagaggatgttgttgataaattattgattcagggcaagttagaccaagagagtaatatagctaaaaagatgtttcacaaaacttacaaaaacaggaattttactgaggaaatcaatgagatacttgtagaggtgaggaaaatccttgaggaaaatcaacatctgtttgaggcaaacccgacgattgatcatcatcagcctgaaaaagttgtccaggaggaacagggttcgtcattggaaaatcacgaagtggttggatttgatgaagaagcaacgaaagtgatcaatcgtctggttgaaggagcagagtgtctagatgttatcccggttgtaggaatgccgggacttggtaaaaccacactggcaagaaaaatctttaatgatcctaagatttcgcgagaatttttcagctacatttgggttttcatcggacaatcaacgtgtgtaaaaagggatatcctttttaatattctgaaagggttcacaaattcatttgatgaattcaaaaacagaaatgaggcaagcataactgatgaaatacgtaagcgtgtggctaatggaggtaaatgtctcattgtcttggatgatgtgtgggatacaaatgttgtagatattgtcaagacagttttccctgataacaaaaaagcccacaggatcatgatgaccactcgacacgaagacattgctagatctgtcaataaatatccccacaatctgaaatttctggatggagacgaaagtttccagctgctagaaaagagagcttttggcgttagccgttgtcctgttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggatttgatatccccgcttggaaattgattcgactctggattgctgagggactcataatgtccaagttgtcgggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacagaactcaattacttgtcgtcgtgtctctattcaatcatctgttcctcaaaacttcatctcaaaaaagacagttgaagaacatgttaggtcattgttatgtttttcctcaaaacaaaaacaagttgacttttctaatatcgacgtcaagctcatccctaccgcatttccacttatgagagtcttagacattgaatccgtcaagtttagtattcccagggaattttaccagctattgcacttgaggtatatcgctatgtcaggtgatttcgagcaacttcctaaactctttacttctttctgtaatgcacaaactctcattctaaatacttccaagcccacccttgatataaaagctgacatatggaacatgccacgtttgcgtcatctgcacaccaacaaacctgcaatcttaccaccccctacaagtagtagtagtagtagtacaaattcttgtttgttgcaaactctatctctggttacaccagaaagctgcaagggaaacgttctttcaaaggctcgtaatgtcaagaaaatgagtgttaaaggtaatttgacaccttttcttgaaactagcaagggtgaatttttcagcaattttcaagtgctaaagctcctggaaagtttaacactgctaaatgatgataagagtaataaatctcttcaccttccgtcagcattctccgaatgtttaccaaatttgaagaagttaactctatcaaaaacaaggtttgactggaatcaggcatatagattagggcaggtgaaaaatctccaggtcctaaaactgaaagaaaatgcattcacggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatctttcccatttggataaccttcaagagatgacgttggaaaacacaagcaaagcaagcaaatctgcaagagaaatagaatgcgagaaaaagaagaagcaagctgatgatccagaaactggcaaattcaagctcactattccctactga

SEQ ID NO: 20 - protein, 889 aa, Solanum lycopersicum
  REGION 1..889: note = Protein NRC6-2a
MDPFTAAALTTAVTATVSLLVENLSHLISYNWKLYTGLKKSCEDLYDEVKRLNAFLVDNANQRSNSTQWDVLVDKIRRTVYKAEDVVDKLLIQGKLDQESNIAKKMFHKTYKNRNFTEEINEILVEVRKILEENQHLFEANPTIDHHQPEKVVQEEQGSSLENHEVVGFDEEATKVINRLVEGAECLDVIPVVGMPGLGKTTLARKIFNDPKISREFFSYIWVFIGQSTCVKRDILFNILKGFTNSFDEFKNRNEASITDEIRKRVANGGKCLIVLDDVWDTNVVDIVKTVFPDNKKAHRIMMTTRHEDIARSVNKYPHNLKFLDGDESFQLLEKRAFGVSRCPVELVEHGEAIVAKCSGVPLTIVVIAGALRGRTSEIDWKVVRENVGKHLIQEDKLQRCVNVVRLSYNHLPQEKKSCFLYFGAFPQGFDIPAWKLIRLWIAEGLIMSKLSGNEIEEIAEYYLNDFANRNLVMVMKKKSNDRIKTCRVHDMLHEFCVEEATRLTLFKQVCLTSDQDIQNSITCRRVSIQSSVPQNFISKKTVEEHVRSLLCFSSKQKQVDFSNIDVKLIPTAFPLMRVLDIESVKFSIPREFYQLLHLRYIAMSGDFEQLPKLFTSFCNAQTLILNTSKPTLDIKADIWNMPRLRHLHTNKPAILPPPTSSSSSSTNSCLLQTLSLVTPESCKGNVLSKARNVKKMSVKGNLTPFLETSKGEFFSNFQVLKLLESLTLLNDDKSNKSLHLPSAFSECLPNLKKLTLSKTRFDWNQAYRLGQVKNLQVLKLKENAFTGPSWRMEPGGFKKLQVLWIEMADFVSWEASNCPFPRLRSLFLISCLNLEAVPLDLSHLDNLQEMTLENTSKASKSAREIECEKKKKQADDPETGKFKLTIPY

SEQ ID NO: 21 - DNA, 8421 bp, Solanum lycopersicum
  misc_feature 1..8421: note = Genomic NRC6-2c
  source 1..8421: mol_type = genomic DNA
agtttgtcaaatcatcaaaactacaaataacagttttgattttggactctcttttttttaaaaaaattttaatattatattatatttttaaattaattttttattggcccacgggccggccctaccaatatttctcaagccccccaaatgagcaggcttattcaggctggacaaaaaaatcattttcttaaatgggttccaaaaatcttagcccaatcctattaaatctcgggttaggccaggccgacccaacggacctagcccatattgacggctctagttagaaccatgaaaattatgtgatatattttggtcaaatgtttctcacaatgaaacttaaaaacctaatgaaggctagcttgaatatatggccatacaacaaaataaaatgacaaaactgatcccttatctttgctagtagtttcaatatagtccttttaagtatcaatcaaatacttttgatcatttttagtaagataaattttgtagataaataacaaattcgttgctaattctgtttagtgataaattatcaaagatattttttaaccacaagcaatttaacgatgaaattcgtacctaattctattttatttttatataaaatattatcgaaactttaatgttttaaattcaatttaaactgtgaaaatatttttaaaaaatctatttattttattcttctatattttgtgctcaatatagttggatgtagttcacacttcacaattgtattacataattttcttatatccttaatctttcttgcattgttttgggaaaaaaatgcaattttataataataattatcgtcataataataataattagtcaagtgaataataaaaataatttctgttaagaattattttaatcaaattatccaccttcttaagaatctcatcatttcaagtaaacgtggatctgccaataagataaaggacctaggaagtgatgataaaaaatatttatttcaaattgggatgatttcaataaacgaataaaattcttgtaacttaggaattaattttttaactttttcttttataacaactctattataagactgaataaaataatgactacattaccccccactatattaattaattctatttaatataaactttacagaaaaacctcggcaaagcgaacaccttgtttcccgtgctcaccaaccacaacaattacgcggttaatatctcaaatggttatacaattttaaaaaattattcggtaaaatcattaaattttattttatattattagagttactcaatttaagtatatttaaatcggtgaaatcactaaactaaatttatcgataaattaagtgttgttaaaattttgttctaaacgactataggaaataggtttctacaaataactgtcggcgcatagtttctttcaaggaaaaatgcagagtgccaagtaaggccgaaaagggtaaaaaattatctataaaataagtccggatgggggaataataagacttgtataagtgtgtctctgaaatttcggacataaattaggagatacttatgtattttcctttgtttcagttatttgtttcacctcttccataatgttaaatccttaattatatatgtctacctctgtacgtcatattatagatagaaaaaaaaattaaatatgtgtctttataatcaacactctatataagtatcactgaaagatctagcgacgttaaatacaactaaatattttttattttgctaccttgaataataattctcaattgtttacaagctttccaacaacacccatttattttcaaccaatgtgaacctctttccaacaacacttaatttttctatcaatatgaacttaattattcaaatgttagctgtcattggattattaaattgttaatgttgtaaggtgatggtgtattatattaagaattaactaataatacttaaagacttggttaatctaattcagagtcaacataaagttaattcattccacatctttatatacacacaactatggcaataacaacatagcaattgtcatctctaaattactactactattcttcttcttcatcaatttaatcctcagaaatggatccgtttacggccgctgccttaaccactgcagtgacagccacggtgagtcttctggtggagaacttgtcgcatcttataagttataattggaagttgtatacaggattgaagaaatcatgcgaagatttgtatgatgaagtgaagcgattaaatgcatttttagccgataacgcgaatcagagaagtaatagtacgcaatgggatgtattagtcgataaaattcgacgtacagtatataaagcagaggatgttgttgataaattattgattcagggcaagttagaccaagagagtaatatagctagaaaaatgtttcacaaaacttacaaaaacaggaattttactgaggaaatcaatgagatacttgtagaggtgaggaaaatccttgatgaaaatcaacatctgtttgaggcaaacccgacgattgatcatcatcagcctgaaaaagttgtccaggaggaacaggtacgtttacacgaaatttcgattttaatgtgatttattgaaatatgaaatgaactatgatttgctcgtttagtaaaactgtataagagttggagaaatagtacttcctctgttttaatatgcattatttgtttgaacttagttaaagtttaatctaaattattcagatctaggacaaggcataagtaaggcaaaggatagaagtacctttctagattatgactcaaattccgtagaattttactttagttaaggtgtgtctctgaaattcagacaattaaaggtcaccaccaaccattattacatgcacaaatcactatcgataattgccaccattggtcaacattatatatcgctgacctccatctatcattaactgtcagtatgattccactattaattatcaaatggtcaccaacaacaaccacaattaatcactaaggccaatcactatactgttcacaaccactatcatctgtcaacactatctaaaatcgatacacacaatcactatcattagtcattaccaccaccgattgtcacattttcttgtagtgaatgatggggattgaaaatatgaagaataaaatagacatgcataggataaggggacatatttttcatagttgaatgggacattgaaaattaataactttcacatgatcataactcatttggtacctgatggaataatttcttgttatgatgatacaatcaagacaagtagccttaataaattgtttaatttgtccatcttacagggttcgtcattggaaaatcacgaagtggttggatttgatgaagaagcaacgaaagtgatcaatcgtctggttgaaggagcagagtgtctagatgttatcccggttgtaggaatgccgggacttggtaaaaccacactggcaagaaaaatctttaatgatcctaagatttcgcgagaatttttcagctacatttgggttttcatcggacaatcaacgtgtgtaaaaagggatatcctttttaatattctgaaagggttcacaaattcatttgatgaattcaaaaacagaaatgaggcaagcataactgatgaaatacgtaagcgtgtggctaatggaggtaaatgtctcattgtcttggatgatgtgtgggatacaaatgttgtagatattgtcaagacagttttccctgataacaaaaaagcccacaggatcatgatgaccactcgacacgaagacattgctagatctgtcaataaatatccccacaatctgaaatttctggatggagatgaaagtttccagctgctagaaaagagagcttttggcgttagccgttgtcctgttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggatttgatatccccgcttggaaattgattcgactctggattgctgagggactcataatgtccaagttgtcgggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacagaactcaattacttgtcgtcgtgtctctattcaatcatctgttcctcaaaacttcatctcaaaaaagacagttgaagaacatgttaggtcattgttatgtttttcctcaaaacaaaaacaagttgacttttctaatatcgacgtcaagctcatccctaccgcatttccacttatgagagtcttagacattgaatccgtcaagtttagtattcccagggaattttaccagctattgcacttgaggtatatcgctatgtcaggtgatttcgagcaacttcctaaactctttacttctttctgtaatgcacaaactctcattctaaatacttccaagcccacccttgatataaaagctgacatatggaacatgccacgtttgcgtcatctgcacaccaacaaacctgcaatcttaccaccccctacaagtagtagtagtagtagtacaaattcttgtttgttgcaaactctatctctggttacaccagaaagctgcaagggaaacgttctttcaaaggctcgtaatgtcaagaaaatgagtgttaaaggtaatttgacaccttttcttgaaactagcaagggtgaatttttcagcaattttcaagtgctaaagctcctggaaagtttaacactgctaaatgatgataagagtaataaatctcttcaccttccgtcagcattctccgaatgtttaccaaatttgaagaagttaactctatcaaaaacaaggtttgactggaatcaggcatatagattagggcaggtgaaaaatctccaggtcctaaaactgaaagaaaatgcattcacggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatctttcccatttggataaccttcaagagatgacgttggaaaacacaagcaaagcaagcaaatctgcaagagaaatagaatgcgagaaaaagaagaagcaagctgatcatccagaaactggcaaattcaagctcactattccctactgaagctgatttcaatgccacaaagtgaagttgttataaggttagtccaattctctccctctcgtatcagtaaatatcgaataagatatttttcaataagattgcatctatccctatagtctctccacgcagaattagtaggtagcagtgtgttgtcttgctgtctcgttcatatcaatagtcatagtattgctcctatagtttcttatccttcgatttttcgttactatacgttgtcccttatacctcgatattagtaatgcttgttgtagtttctcttatgttactatctgctgtttttgttattacctgttgtttcttgtatttccgtcatttctttttgggactcctttgaactttctccttgagacaaaggtcaatcggaaacaacctacctctctttctttgagataaggttcctccccagactctattttgtgggactacactgagtatattgttttgggtttcgcacttcactgaaacattaaattgggcaacattactctgttatactaataatacagattttaggtgccttgatatcaaacatagagtaaattcaattattttcgtgttcatcacattcctcatctatttttctcttacttattttcctcctttttagcaggagagtatcgaggcatggtgatgcatttgcagtctcagttttgattacctgttccaataaagaaacttgttacaagtatgttttgttgattcaaacatgccaagtttgaaaacgaagttttatattaatgtttttgcgttttctttcagtgctgaataatttgtctaatctgtagttgatgtttggatatctatgtattgttcgattgacacgagttgtttatactatcatttgttgtttatgaatatgtaaaacatatttgatttatgtttgcaacgtcaacgtgaacattcaattagtctaatcaaagtctagatctaaaattgattaatttaagtttataatttattttgaattattatctaaaaccgaagactttctactcgaaaatcgaaatcatagctgacagtctccataaatatatttgcaattgaataacataataagcatttatcagttaattttatggagtttttggatttacgtgatattcaaacatctcccgcgcatattaattatgtgaagtaatgaagtgtgtaacataagtcaaaaggtgcataaataaagagtcaagggtaataagactttagtttactttaggtgtgtctctgaaatttcgattataatctacgagatattgtgtcttgtcccaacatttaaataaaatgaacaaataattttaattaccaaatggatccatagtctgtttagttgactaatggaaagatttataggaaggtgagattgttttatccctgattagaggttttggttcgagttttgtgtatacgaaaaaattgtgttgaaagcgtcacttatgaataggccctataatgtatgattcgaatttagtctgagttctaatatgaattttgaacacattgaaaaattatatcctttgttaaatttcatggttttaacatcttggaagtaaagaaaaaggtaaaatattttttttttctatttcatttttttacatgtaatatttgaattcaaaggcagagagagtaataccccccttttatttttatttgtggattacatatatacataagaactagaaaaatgtttatattttcgacttgacaatgatagaatgtaaatgttggatatatagaataatttgttggagcttctaccattttttgcttaccgtcttaaggttgaaggcgtagatgtttattatccgaataattcctttttgtcgataatcaagcatcaatattagattagtagccgaccctcttctcttacctctctaaacaatttttcaatatttccattttggttggtaaaaatattagagaacattttctctatagaaatatcatgtgtttattcagaattgttatttactacatatatttttaataattaaaaacctataaatttaatagtttacattcacaattattgcttgttgctgttcttttgccttgtgtttctaatttttaccgttattaaatatgcatgtgattagttgaaatatcaaatctcatattaaatggaaaaatgaaagtcaacgaagcatggtagctgtcaaatacgctccaactcaaagcattactgtcatgatggatttcctattgatccatcgtgaaaaattgaacgatatgttggcacatgtcagaatgcttaaaatatcgaattaatataattgaatttaactttgaaaattaatcaaattaactttcgaaaagcgctacctgacaattaaaaatagaaggagtgagcagtgaacttaaactaatctatttcagatctctagagtttgataaactaattgacagttcaacatttgcagatctaacttcaacatttgcttcttgtaaatctgcaacaaggcaaactaccatttgagagtaaagaaaaaggtataatataatgtttttctagttttcattttacatgtatttttgtgattcacttgatttgaatgaaacatacattacaagaactagaaaacgaatttgtatttttgacttagataattataaaatgttagatgattgtcataatttgtagtattcactcttgtttttagtaaatatactctttcttaaatgtgattgatttacagattatatattttagaatttttgcttattaaaagaaaaattaattagtggcggaaatttgaggtgaattcgtgaagaatggaagaagttggaaatttgagagttttacatgtgttttttaaggtagaagaaacaaaaagaatccatttgatctaaaattccaattgaaaagtattaaaagttgtttgggatct

SEQ ID NO: 22 - DNA, 2670 bp, Solanum lycopersicum
  misc_feature 1..2670: note = CDS NRC6-2c
  source 1..2670: mol_type = genomic DNA
atggatccgtttacggccgctgccttaaccactgcagtgacagccacggtgagtcttctggtggagaacttgtcgcatcttataagttataattggaagttgtatacaggattgaagaaatcatgcgaagatttgtatgatgaagtgaagcgattaaatgcatttttagccgataacgcgaatcagagaagtaatagtacgcaatgggatgtattagtcgataaaattcgacgtacagtatataaagcagaggatgttgttgataaattattgattcagggcaagttagaccaagagagtaatatagctagaaaaatgtttcacaaaacttacaaaaacaggaattttactgaggaaatcaatgagatacttgtagaggtgaggaaaatccttgatgaaaatcaacatctgtttgaggcaaacccgacgattgatcatcatcagcctgaaaaagttgtccaggaggaacagggttcgtcattggaaaatcacgaagtggttggatttgatgaagaagcaacgaaagtgatcaatcgtctggttgaaggagcagagtgtctagatgttatcccggttgtaggaatgccgggacttggtaaaaccacactggcaagaaaaatctttaatgatcctaagatttcgcgagaatttttcagctacatttgggttttcatcggacaatcaacgtgtgtaaaaagggatatcctttttaatattctgaaagggttcacaaattcatttgatgaattcaaaaacagaaatgaggcaagcataactgatgaaatacgtaagcgtgtggctaatggaggtaaatgtctcattgtcttggatgatgtgtgggatacaaatgttgtagatattgtcaagacagttttccctgataacaaaaaagcccacaggatcatgatgaccactcgacacgaagacattgctagatctgtcaataaatatccccacaatctgaaatttctggatggagatgaaagtttccagctgctagaaaagagagcttttggcgttagccgttgtcctgttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggatttgatatccccgcttggaaattgattcgactctggattgctgagggactcataatgtccaagttgtcgggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacagaactcaattacttgtcgtcgtgtctctattcaatcatctgttcctcaaaacttcatctcaaaaaagacagttgaagaacatgttaggtcattgttatgtttttcctcaaaacaaaaacaagttgacttttctaatatcgacgtcaagctcatccctaccgcatttccacttatgagagtcttagacattgaatccgtcaagtttagtattcccagggaattttaccagctattgcacttgaggtatatcgctatgtcaggtgatttcgagcaacttcctaaactctttacttctttctgtaatgcacaaactctcattctaaatacttccaagcccacccttgatataaaagctgacatatggaacatgccacgtttgcgtcatctgcacaccaacaaacctgcaatcttaccaccccctacaagtagtagtagtagtagtacaaattcttgtttgttgcaaactctatctctggttacaccagaaagctgcaagggaaacgttctttcaaaggctcgtaatgtcaagaaaatgagtgttaaaggtaatttgacaccttttcttgaaactagcaagggtgaatttttcagcaattttcaagtgctaaagctcctggaaagtttaacactgctaaatgatgataagagtaataaatctcttcaccttccgtcagcattctccgaatgtttaccaaatttgaagaagttaactctatcaaaaacaaggtttgactggaatcaggcatatagattagggcaggtgaaaaatctccaggtcctaaaactgaaagaaaatgcattcacggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatctttcccatttggataaccttcaagagatgacgttggaaaacacaagcaaagcaagcaaatctgcaagagaaatagaatgcgagaaaaagaagaagcaagctgatcatccagaaactggcaaattcaagctcactattccctactga

SEQ ID NO: 23 - protein, 889 aa, Solanum lycopersicum
  REGION 1..889: note = Protein NRC6-2c
MDPFTAAALTTAVTATVSLLVENLSHLISYNWKLYTGLKKSCEDLYDEVKRLNAFLADNANQRSNSTQWDVLVDKIRRTVYKAEDVVDKLLIQGKLDQESNIARKMFHKTYKNRNFTEEINEILVEVRKILDENQHLFEANPTIDHHQPEKVVQEEQGSSLENHEVVGFDEEATKVINRLVEGAECLDVIPVVGMPGLGKTTLARKIFNDPKISREFFSYIWVFIGQSTCVKRDILFNILKGFTNSFDEFKNRNEASITDEIRKRVANGGKCLIVLDDVWDTNVVDIVKTVFPDNKKAHRIMMTTRHEDIARSVNKYPHNLKFLDGDESFQLLEKRAFGVSRCPVELVEHGEAIVAKCSGVPLTIVVIAGALRGRTSEIDWKVVRENVGKHLIQEDKLQRCVNVVRLSYNHLPQEKKSCFLYFGAFPQGFDIPAWKLIRLWIAEGLIMSKLSGNEIEEIAEYYLNDFANRNLVMVMKKKSNDRIKTCRVHDMLHEFCVEEATRLTLFKQVCLTSDQDIQNSITCRRVSIQSSVPQNFISKKTVEEHVRSLLCFSSKQKQVDFSNIDVKLIPTAFPLMRVLDIESVKFSIPREFYQLLHLRYIAMSGDFEQLPKLFTSFCNAQTLILNTSKPTLDIKADIWNMPRLRHLHTNKPAILPPPTSSSSSSTNSCLLQTLSLVTPESCKGNVLSKARNVKKMSVKGNLTPFLETSKGEFFSNFQVLKLLESLTLLNDDKSNKSLHLPSAFSECLPNLKKLTLSKTRFDWNQAYRLGQVKNLQVLKLKENAFTGPSWRMEPGGFKKLQVLWIEMADFVSWEASNCPFPRLRSLFLISCLNLEAVPLDLSHLDNLQEMTLENTSKASKSAREIECEKKKKQADHPETGKFKLTIPY

SEQ ID NO: 24 - DNA, 2673 bp, Solanum lycopersicum
  misc_feature 1..2673: note = CDS NRC6-2b
  source 1..2673: mol_type = genomic DNA
atggatccgtttacggcggctgcattaaccactgcggtgacagccacggtgagccttctggtggagaacttgtcgcatcttataagttataattggaagttgtatacaggattgaagaaatcatgcgaagatttgtatgatgaagtgaagcgattaaatgcatttttagtcgataacgcgaatcagagaagtaatagtacgcaatgggatgtattagtcgataaaattcgacgtacagtatataaagcagaggatgttgttgataaattattgattcagggcaagttagaccaagagagtaatatagctaaaaagatgtttcacaaaacttacaaaaacaggaattttactgaggaaatcaatgagatacttgtagaggtgaggaaaatccttgaggaaaatcaacatctgtttgaggcaaacccgacgattgatcatcatcagcctgaaaaagttgtccaggaggaacagggttcgtcattggaaaatcacgaagtggttggatttgatgaagaagcaacgaaagtgatcaatcgtctggttgaaggagcagagtgtctagatgttatcccggttgtaggaatgccgggacttggtaaaaccacactggcaagaaaaatctttaatgatcctaagatttcgcgagaatttttcagctacatttgggttttcatcggacaatcaacgtgtgtaaaaagggatatcctttttaatattctgaaagggttcacaaattcatttgatgaattcaaaaacagaaatgaggcaagcataactgatgaaatacgtaagcgtgtggctaatggaggtaaatgtctcattgtcttggatgatgtgtgggatacaaatgttgtagatattgtcaagacagttttccctgataacaaaaaagcccacaggatcatgatgaccactcgacacgaagacattgctagatctgtcaataaatatccccacaatctgaaatttctggatggagatgaaagtttccagctgctagaaaagagagcttttggcgttagccgttgtcctgttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggatttgatatccccgcttggaaattgattcgactctggattgctgagggactcataatgtccaagttgtcgggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacagaactcaattacttgtcgtcgtgtctctattcaatcatctgttcctcaaaacttcatctcaaaaaagacagttgaagaacatgttaggtcattgttatgtttttcctcaaaacaaaaacaagttgacttttctaatatcgacgtcaagctcatccctaccgcatttccacttatgagagtcttagacattgaatccgtcaagtttagtattcccagggaattttaccagctattgcacttgaggtatatcgctatgtcaggtgatttcgagcaacttcctaaactctttacttctttctgtaatgcacaaactctcattctaaatacttccaagcccacccttgatataaaagctgacatatggaacatgccacgtttgcgtcatctgcacaccaacaaacctgcaatcttaccaccccctacaagtagtagtagtagtagtagtacaaattcttgtttgttgcaaactctatctctggttacaccagaaagctgcaagggaaacgttctttcaaaggctcgtaatgtcaaaaaaatgagtgttaaaggtaatttgacgccttttcttgaaactagcaagggtgaatttttcagcaattttcaagtgctaaagctcctggaaagtttaacactgctaaatgatgataagagtaataaatctcttcaccttccgtcagcattctccgaatgtttaccaaatttgaagaagttaactctatcaaaaacaaggtttgactggaatcaggcatatagattagggcaggtgaaaaatctccaggtcctaaaactgaaagaaaatgcattcacggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatctttcccatttggataaccttcaagagatgacgttggaaaacacaagcaaagcaagcaaatctgcaagagaaatagaatgcgagaaaaagaagaagcaagctgatcatccagaaactggcaaattcaagctcactattccctactga

SEQ ID NO: 25 - protein, 890 aa, Solanum lycopersicum
  REGION 1..890: note = Protein NRC6-2b
MDPFTAAALTTAVTATVSLLVENLSHLISYNWKLYTGLKKSCEDLYDEVKRLNAFLVDNANQRSNSTQWDVLVDKIRRTVYKAEDVVDKLLIQGKLDQESNIAKKMFHKTYKNRNFTEEINEILVEVRKILEENQHLFEANPTIDHHQPEKVVQEEQGSSLENHEVVGFDEEATKVINRLVEGAECLDVIPVVGMPGLGKTTLARKIFNDPKISREFFSYIWVFIGQSTCVKRDILFNILKGFTNSFDEFKNRNEASITDEIRKRVANGGKCLIVLDDVWDTNVVDIVKTVFPDNKKAHRIMMTTRHEDIARSVNKYPHNLKFLDGDESFQLLEKRAFGVSRCPVELVEHGEAIVAKCSGVPLTIVVIAGALRGRTSEIDWKVVRENVGKHLIQEDKLQRCVNVVRLSYNHLPQEKKSCFLYFGAFPQGFDIPAWKLIRLWIAEGLIMSKLSGNEIEEIAEYYLNDFANRNLVMVMKKKSNDRIKTCRVHDMLHEFCVEEATRLTLFKQVCLTSDQDIQNSITCRRVSIQSSVPQNFISKKTVEEHVRSLLCFSSKQKQVDFSNIDVKLIPTAFPLMRVLDIESVKFSIPREFYQLLHLRYIAMSGDFEQLPKLFTSFCNAQTLILNTSKPTLDIKADIWNMPRLRHLHTNKPAILPPPTSSSSSSSTNSCLLQTLSLVTPESCKGNVLSKARNVKKMSVKGNLTPFLETSKGEFFSNFQVLKLLESLTLLNDDKSNKSLHLPSAFSECLPNLKKLTLSKTRFDWNQAYRLGQVKNLQVLKLKENAFTGPSWRMEPGGFKKLQVLWIEMADFVSWEASNCPFPRLRSLFLISCLNLEAVPLDLSHLDNLQEMTLENTSKASKSAREIECEKKKKQADHPETGKFKLTIPY

SEQ ID NO: 26 - DNA, 2673 bp, Solanum lycopersicum
  misc_feature 1..2673: note = CDS NRC6-3
  source 1..2673: mol_type = genomic DNA
atggatccgtttacggccgctgccttaaccactgcagtgacagccacggtgagtcttctggtggagaacttgtcgcatcttataagttataattggaagttgtatacaggattgaagaaatcatgcgaagatttgtatgatgaagtgaagcgattaaatgcatttttagtcgataacgcgaatcagagaagtaatagtacgcaatgggatgtattagtcgataaaattcgacgtacagtatataaagcagaggatgttgttgataaattattgattcagggcaagttagaccaagagagtaatatagctaaaaagatgtttcacaaaacttacaaaaacaggaattttactgaggaaatcaatgagatacttgtagaggtgaggaaaatccttgaggaaaatcaacatctgtttgaggcaaacccgacgattgatcatcatcagcctgaaaaagttgtccaggaggaacagggttcatcgttggaaaatcacgaagtggtaggatttgatgaagaagcaacgaaagtgatcaatcgtctggttgaaggagcagagcgtctagatgttatcccggttgtaggaatgccgggacttggtaaaaccacactggcaagaaaaatctttaatgatccgaagatttcgcgagaatttttcagctacatttgggttttcatcggacaatcaacgtgtgtaaaaagggatatcctttttaatattctgaaagggttcacaaattcatttgatgaattcaaaaacagaaatgaggcagacataactgatgaaatacgtaagcgtgtggctaatggaggtaaatgtctcattgtcttggatgatgtgtgggatccaaatgttgtagatattgtcaagacagttttccctgataacaaaaaagcccacaggatcatgatgaccactcgacacgaagacattgctagatctgtcaataaatatcctcacaatctgaaatttctggatggagatgaaagtttccagctgctagaaaagagagcttttggcgttagccgttgtccagttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggattcgatatccccgcttggaagttgattcgactctggattgctgagggactcataatgtccaagttgtcaggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacaaaactcaattgcttgtcgtcgtgtctctattcaatcatctgttcctcaaaacttcatctcaaaaaagacagttgaagagcatgttaggtcatttttatgtttttcctcaaaacaaaaacaagttgacttttctaatatcgacgtcaagctcatccctgccgcatttccacttatgagagtcttagacattgaatccgtcaagtttagtattcccagggaattttaccagctattgcacttgaggtatatcgctatctcaggtgatttcgagcaacttcctaaactctttacttctttctgtaatgcacaaactctcattctaaatacttccaagcccacccttgatataaaagctgacatatggaacatgccacgtttgcgtcatctgcgcaccaacaaacctgcaatcttaccaccccctacaagtagtagtagtagtagtagtacaaattcttgttttttgcaaactctatctctggttacaccagaaagctgcaagggaaatgttctttcaaaagctcgtaatgtcaaaaaaatgagtgttaaaggtaatttgacgccttttcttgaaactagcaagggtgaatttttcagcaattttcaagtgctaaagctcctggaaagtttaacactgctaaatgatgataagagtaacaaatctcttcaccttccgtcagcattctccgaatgtttaccaaatttgaagaagttaactctatcaaaaacaaggtttgactggaatcaggcatatagattggggcaggtgaaaaatctccaggtcctaaaactgaaagaaaatgcattcatggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatcttgcccatttggataaccttcaagagatgacgttggaaaacacaagcaaagcaagcaaatctgcaagagaaatagaatgcgagaaaaagaagaagcaagctgatcatccagaaagtggcaaattcaagctcactattccctactga

SEQ ID NO: 27 - protein, 890 aa, Solanum lycopersicum
  REGION 1..890: note = Protein NRC6-3
MDPFTAAALTTAVTATVSLLVENLSHLISYNWKLYTGLKKSCEDLYDEVKRLNAFLVDNANQRSNSTQWDVLVDKIRRTVYKAEDVVDKLLIQGKLDQESNIAKKMFHKTYKNRNFTEEINEILVEVRKILEENQHLFEANPTIDHHQPEKVVQEEQGSSLENHEVVGFDEEATKVINRLVEGAERLDVIPVVGMPGLGKTTLARKIFNDPKISREFFSYIWVFIGQSTCVKRDILFNILKGFTNSFDEFKNRNEADITDEIRKRVANGGKCLIVLDDVWDPNVVDIVKTVFPDNKKAHRIMMTTRHEDIARSVNKYPHNLKFLDGDESFQLLEKRAFGVSRCPVELVEHGEAIVAKCSGVPLTIVVIAGALRGRTSEIDWKVVRENVGKHLIQEDKLQRCVNVVRLSYNHLPQEKKSCFLYFGAFPQGFDIPAWKLIRLWIAEGLIMSKLSGNEIEEIAEYYLNDFANRNLVMVMKKKSNDRIKTCRVHDMLHEFCVEEATRLTLFKQVCLTSDQDIQNSIACRRVSIQSSVPQNFISKKTVEEHVRSFLCFSSKQKQVDFSNIDVKLIPAAFPLMRVLDIESVKFSIPREFYQLLHLRYIAISGDFEQLPKLFTSFCNAQTLILNTSKPTLDIKADIWNMPRLRHLRTNKPAILPPPTSSSSSSSTNSCFLQTLSLVTPESCKGNVLSKARNVKKMSVKGNLTPFLETSKGEFFSNFQVLKLLESLTLLNDDKSNKSLHLPSAFSECLPNLKKLTLSKTRFDWNQAYRLGQVKNLQVLKLKENAFMGPSWRMEPGGFKKLQVLWIEMADFVSWEASNCPFPRLRSLFLISCLNLEAVPLDLAHLDNLQEMTLENTSKASKSAREIECEKKKKQADHPESGKFKLTIPY

SEQ ID NO: 28 - DNA, 2673 bp, Solanum lycopersicum
  misc_feature 1..2673: note = NRC6-1a
  source 1..2673: mol_type = genomic DNA
atggatccgtttacggccgctgcattaaccactgcggtgacagccacggtgagccttctggtggagaacttgtcgcatcttataagttataattggaagttgtatacaggattgaagaaatcatgcgaagatttgtatgatgaagtgaagcgattaaatgcatttttagtcgataacgcgaatcagagaagtaatagtacgcaatgggatgtactagtcgataaaattcgacgtacagtatataaagcagaggatgttgttgataaattattgattcagggcaagttagaccaagagagtaatatagctaaaaagatgtttcacaaaacttacaaaaacaggaattttactgaggaaatcaatgagatacttgtagaggtgaggaaaatccttgaggaaaatcaacatctgtttgaggcaaacccgacgattgatcatcatcagcctgaaaaagttgtccaggaggaacagggttcgtcattggaaaatcacgaagtggttggatttgatgaagaagcaacgaaagtgatcaatcgtctggttgaaggagcagagtgtctagatgttatcccggttgtaggaatgccgggacttggtaaaaccacactggcaagaaaaatctttaatgatcctaagatttcgcgagaatttttcagctacatttgggttttcatcggacaatcaacgtgtgtaaaaagggatatcctttttaatattctgaaagggttcacaaattcatttgatgaattcaaaaacagaaatgaggcaagcataactgatgaaatacgtaagcgtgtggctaatggaggtaaatgtctcattgtcttggatgatgtgtgggatccaaatgttgtagatattgtcaagacagttttccctgataacaaaaaagcccacaggatcatgatgaccactcgacacgaagacattgctagatctgtcaataaatatccccacaatctgaaatttctggatggagatgaaagtttccagctgctagaaaagagagcttttggcgttagccgttgtcctgttgagttagtagaacatggagaagccattgtagcaaaatgtagtggagtaccacttacaattgtggtaattgcaggagctttaagaggtcgtacgagtgaaattgattggaaagtagttagggaaaatgtggggaagcatcttatacaagaagacaaacttcagagatgtgtgaatgttgtgagattgagttacaatcatttgcctcaagaaaaaaaatcttgcttcttgtattttggtgcctttcctcaaggatttgatatccccgcttggaaattgattcgactctggattgctgagggactcataatgtccaagttgtcaggcaacgaaattgaagagatagcagagtattatcttaatgactttgccaacaggaacttagtgatggtgatgaaaaagaaatctaatgatcgaataaaaacatgtcgtgttcacgacatgttacatgagttttgcgttgaagaggctactagattgactcttttcaaacaagtatgtctcacatctgatcaagacatacagaactcaattacttgtcgtcgtgtctctattcaatcatctgttcctcaaaacttcatctcaaaaaagacagttgaagaacatgttaggtcattgttatgtttttcctcaaaacaaaaacaagttgacttttctaatatcgacgtcaagctcatccctaccgcatttccacttatgagagtcttagacattgaatccgtcaagtttagtattaccagggaattttaccagctattgcacttgaggtatatcgctatctcaggtgatttcgagcaacttcctaaactctttacttctttctgtaatgcacaaactctcattctaaatacttccaagcccacccttgatataaaagctgacatatggaacatgccacgtttgcgtcatctgcgcaccaacaaacctgcaatcttaccaccccctacaagtagtagtagtagtagtagtacaaattcttgttttttgcaaactctatctctggttacaccagaaagctgcaagggaaatgttctttcaaaagctcgtaatgtcaaaaaaatgagtgttaaaggtaatttgacgccttttcttgaaactagcaagggtgaatttttcagcaattttcaagtgctaaagctcctggaaagtttaacactgctaaatgatgataagagtaacaaatctcttcaccttccgtcagcattctccgaatgtttaccaaatttgaagaagttaactctatcaaaaacaaggtttgactggaatcaggcatatagattggggcaggtgaaaaatctccaggtcctaaaaatgaaagaaaatgcattcatggggccgtcctggaggatggagccaggaggtttcaagaaacttcaggtcttgtggattgaaatggcagatttcgtgtcgtgggaggcatcaaactgtcctttcccaagacttaggagccttttcctgatctcctgtcttaatcttgaggctgtgccactcgatcttgcccatttggataaccttcaagagatgacgttggaaaacacaagcaaagcaagcaaatctgcaagagaaatagaatgcgagaaaaagaagaagcaagctgatcatccagaaagtggcaaattcaagctcactattccctactga

SEQ ID NO: 29 - protein, 890 aa, Solanum lycopersicum
  REGION 1..890: note = Protein NRC6-1a
MDPFTAAALTTAVTATVSLLVENLSHLISYNWKLYTGLKKSCEDLYDEVKRLNAFLVDNANQRSNSTQWDVLVDKIRRTVYKAEDVVDKLLIQGKLDQESNIAKKMFHKTYKNRNFTEEINEILVEVRKILEENQHLFEANPTIDHHQPEKVVQEEQGSSLENHEVVGFDEEATKVINRLVEGAECLDVIPVVGMPGLGKTTLARKIFNDPKISREFFSYIWVFIGQSTCVKRDILFNILKGFTNSFDEFKNRNEASITDEIRKRVANGGKCLIVLDDVWDPNVVDIVKTVFPDNKKAHRIMMTTRHEDIARSVNKYPHNLKFLDGDESFQLLEKRAFGVSRCPVELVEHGEAIVAKCSGVPLTIVVIAGALRGRTSEIDWKVVRENVGKHLIQEDKLQRCVNVVRLSYNHLPQEKKSCFLYFGAFPQGFDIPAWKLIRLWIAEGLIMSKLSGNEIEEIAEYYLNDFANRNLVMVMKKKSNDRIKTCRVHDMLHEFCVEEATRLTLFKQVCLTSDQDIQNSITCRRVSIQSSVPQNFISKKTVEEHVRSLLCFSSKQKQVDFSNIDVKLIPTAFPLMRVLDIESVKFSITREFYQLLHLRYIAISGDFEQLPKLFTSFCNAQTLILNTSKPTLDIKADIWNMPRLRHLRTNKPAILPPPTSSSSSSSTNSCFLQTLSLVTPESCKGNVLSKARNVKKMSVKGNLTPFLETSKGEFFSNFQVLKLLESLTLLNDDKSNKSLHLPSAFSECLPNLKKLTLSKTRFDWNQAYRLGQVKNLQVLKMKENAFMGPSWRMEPGGFKKLQVLWIEMADFVSWEASNCPFPRLRSLFLISCLNLEAVPLDLAHLDNLQEMTLENTSKASKSAREIECEKKKKQADHPESGKFKLTIPY

SEQ ID NO: 30 - protein, 4 aa, Solanum lycopersicum
  REGION 1..4: note = N-terminal motif
MADA

SEQ ID NO: 31 - DNA, 23 bp, Lactuca sativa
  misc_feature 1..23: note = Forward primer
  source 1..23: mol_type = genomic DNA
aggaatgtataaatactctgaca

SEQ ID NO: 32 - DNA, 20 bp, Lactuca sativa
  misc_feature 1..20: note = Reverse primer
  source 1..20: mol_type = genomic DNA
gcctagcaactaatgtatct

(19) United States
(12) Patent Application Publication
(10) Pub. No.: US 2026/0215388 A1
(43) Pub. Date: Jul. 30, 2026

(54) ROOT-KNOT NEMATODE RESISTANCE CONFERRING GENE

(71) Applicant: RIJK ZWAAN ZAADTEELT EN ZAADHANDEL B.V., De Lier (NL)

(72) Inventors: Raoul Jacobus Johannes Maria FRIJTERS, De Lier (NL); Jonathan KALISVAART, De Lier (NL); Adriaan VERHAGE, De Lier (NL)

(21) Appl. No.: 19/424,785

(22) Filed: Dec. 18, 2025

Related U.S. Application Data
(62) Division of application No. 18/064,456, filed on Dec. 12, 2022, now Pat. No. 12,588,644.
Application No. 18/064,456 is a continuation-in-part of application No. PCT/EP2021/066682, filed on Jun. 18, 2021.

(30) Foreign Application Priority Data
Jun. 18, 2020 (WO) PCT/EP2020/067028

(51) Int. Cl. A01H 6/82; A01H 1/00

(52) U.S. Cl. CPC A01H 6/825; A01H 1/1265

(57) ABSTRACT

A nucleic acid encoding an MeR1 protein, which confers resistance to root-knot nematode when present in a Solanaceae plant is provided. Said nucleic acid has a nucleotide sequence that may comprise a coding sequence according to SEQ ID NO: 2, 3, 4, 5, 6, 7, 8 or 9, or a coding sequence having at least 90% sequence identity with SEQ ID NO: 2, 3, 4, 5, 6, 7, 8 or 9; or a nucleotide sequence encoding a protein having an amino acid sequence according to SEQ ID NO: 10, 11, 12, 13, 14, 15, 16 or 17, or a protein having an amino acid sequence having at least 85% sequence identity with SEQ ID NO: 10, 11, 12, 13, 14, 15, 16 or 17. The invention further relates to plants and seeds which may comprise the nucleic acid, methods for making and identifying such plants and use of the nucleic acid.

### RELATED APPLICATIONS AND INCORPORATION BY REFERENCE

This application is a divisional of U.S. application Ser. No. 18/064,456 filed Dec. 12, 2022, now allowed, which is a continuation-in-part application of international patent application Serial No. PCT/EP2021/066682 filed 18 Jun. 2021, which published as PCT Publication No. WO 2021/255272 on 23 Dec. 2021, which claims benefit of international patent application Serial No. PCT/EP2020/067028 filed 18 Jun. 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing, which has been submitted electronically and is hereby incorporated by reference in its entirety. Said XML copy, was created Dec. 9, 2022, is named Y7954_00560SL.xml and is 120,906 bytes in size.

### FIELD OF THE INVENTION

The present invention relates to a nucleic acid molecule providing resistance to a root-knot nematode and to a root-knot nematode resistant plant of the Solanaceae family which may comprise the nucleic acid molecule(s), and a method for the identification of the presence of the nucleic acid molecule(s) in a plant. The invention further relates to a method for producing such plant and methods for identification and selection of such a plant. The invention also relates to a seed of the root-knot nematode resistant plant of the Solanaceae family.

### BACKGROUND OF THE INVENTION

One of the problems that is encountered when growing plants of the Solanaceae family is the occurrence of various plant parasitic nematodes. A number of resistance genes against some nematode species have been identified, which resistances are incorporated in suitable varieties through breeding. This allows the growers to obtain a good yield even when a certain nematode is present during production.

Regularly, however, new nematodes or strains of known nematodes are identified, that in certain instances can break the available resistance. For instance in tomato (Solanum lycopersicum) the known Mi1-2 resistance gene confers resistance against some nematodes, but not against the root-knot nematode Meloidogyne enterolobii (M. enterolobii).

Citation or identification of any document in this application is not an admission that such document is available as prior art to the present invention.

### SUMMARY OF THE INVENTION

The nematodes to which the invention relates use the plant root for their life and therefore the infection by root-knot nematodes such as M. enterolobii leads to root galls that can lead to the death of the infected plant or to a reduction of yield.

Surprisingly, it was found after extensive research that an NBS-LRR gene, herein called MeR1 (Meloidogyne enterolobii resistant 1) is expressed in the root and is responsible for the resistance to a root-knot nematode in a plant of the Solanaceae family.

The invention relates to a nucleic acid molecule encoding an MeR1 protein, which confers resistance to root-knot nematode, in particular to M. enterolobii, when present in a plant of the Solanaceae family, in particular in a Solanum lycopersicum plant, wherein said nucleic acid molecule has a nucleotide sequence which is:
a) a nucleotide sequence which may comprise a coding sequence according to SEQ ID NO: 2, 3, 4, 5, 6, 7, 8 or 9, or a coding sequence which has in order of increased preference 90%, 91%, 92%, 93%, 94%, 95%, 96%, 97%, 98%, 99% sequence identity with the sequence according to SEQ ID NO: 2, 3, 4, 5, 6, 7, 8 or 9; or
b) a nucleotide sequence encoding a protein having an amino acid sequence according to SEQ ID NO: 10, 11, 12, 13, 14, 15, 16 or 17, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 10, 11, 12, 13, 14, 15, 16 or 17.

The invention relates in particular to a nucleic acid molecule encoding an MeR1 protein, which confers resistance to root-knot nematode, in particular to M. enterolobii, when present in a plant of the Solanaceae family, in particular in a Solanum lycopersicum plant, wherein said nucleic acid molecule has a nucleotide sequence which is:
a) a nucleotide sequence which may comprise a coding sequence according to SEQ ID NO: 2, or a coding sequence which has in order of increased preference 90%, 91%, 92%, 93%, 94%, 95%, 96%, 97%, 98%, 99% sequence identity with the sequence according to SEQ ID NO: 2; or
b) a nucleotide sequence encoding a protein having an amino acid sequence according to SEQ ID NO: 10, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 10; or
a) a nucleotide sequence which may comprise a coding sequence according to SEQ ID NO: 3, or a coding sequence which has in order of increased preference 90%, 91%, 92%, 93%, 94%, 95%, 96%, 97%, 98%, 99% sequence identity with the sequence according to SEQ ID NO: 3; or
b) a nucleotide sequence encoding a protein having an amino acid sequence according to SEQ ID NO: 11, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 11; or
a) a nucleotide sequence which may comprise a coding sequence according to SEQ ID NO: 4, or a coding sequence which has in order of increased preference 90%, 91%, 92%, 93%, 94%, 95%, 96%, 97%, 98%, 99% sequence identity with the sequence according to SEQ ID NO: 4; or
b) a nucleotide sequence encoding a protein having an amino acid sequence according to SEQ ID NO: 12, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 12; or
a) a nucleotide sequence which may comprise a coding sequence according to SEQ ID NO: 5, or a coding sequence which has in order of increased preference 90%, 91%, 92%, 93%, 94%, 95%, 96%, 97%, 98%, 99% sequence identity with the sequence according to SEQ ID NO: 5; or
b) a nucleotide sequence encoding a protein having an amino acid sequence according to SEQ ID NO: 13, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 13; or
a) a nucleotide sequence which may comprise a coding sequence according to SEQ ID NO: 6, or a coding sequence which has in order of increased preference 90%, 91%, 92%, 93%, 94%, 95%, 96%, 97%, 98%, 99% sequence identity with the sequence according to SEQ ID NO: 6; or
b) a nucleotide sequence encoding a protein having an amino acid sequence according to SEQ ID NO: 14, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 14; or
a) a nucleotide sequence which may comprise a coding sequence according to SEQ ID NO: 7, or a coding sequence which has in order of increased preference 90%, 91%, 92%, 93%, 94%, 95%, 96%, 97%, 98%, 99% sequence identity with the sequence according to SEQ ID NO: 7; or
b) a nucleotide sequence encoding a protein having an amino acid sequence according to SEQ ID NO: 15, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 15; or
a) a nucleotide sequence which may comprise a coding sequence according to SEQ ID NO: 8, or a coding sequence which has in order of increased preference 90%, 91%, 92%, 93%, 94%, 95%, 96%, 97%, 98%, 99% sequence identity with the sequence according to SEQ ID NO: 8; or
b) a nucleotide sequence encoding a protein having an amino acid sequence according to SEQ ID NO: 16, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 16; or
a) a nucleotide sequence which may comprise a coding sequence according to SEQ ID NO: 9, or a coding sequence which has in order of increased preference 90%, 91%, 92%, 93%, 94%, 95%, 96%, 97%, 98%, 99% sequence identity with the sequence according to SEQ ID NO: 9; or
b) a nucleotide sequence encoding a protein having an amino acid sequence according to SEQ ID NO: 17, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 17.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSIT

Seed of tomato Solanum lycopersicum comprising the nucleic acid molecules of the invention homozygously, resulting in a M. enterolobii resistant plant, was deposited with NCIMB Ltd, Ferguson Building, Craibstone Estate, Bucksburn, Aberdeen AB21 9YA, UK on Nov. 18, 2019, under deposit accession numbers NCIMB 43515.

The Deposits with NCIMB Ltd, under deposit accession number NCIMB 43515 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Results of the M. enterolobii disease testing of plants with a modified MeR1 gene of the invention; Amount of root knots on the source, susceptible line 1 and 2 and the mutated lines with the deletion leading to a non-functional MeR1 gene (4) and without the deletion with a functional MeR1 gene (+).

### DETAILED DESCRIPTION OF THE INVENTION

During the research leading to the invention it has been found that the MeR1 gene which may comprise the nucleic acid molecule having the genomic sequence according to SEQ ID NO: 1 was found in wild tomato species, in particular in Solanum pimpinellifolium species. When this nucleic acid molecule was transferred to a root-knot nematode susceptible Solanum lycopersicum plant, the Solanum lycopersicum plant became resistant to the root-knot nematode, in particular to M. enterolobii. Therefore, the MeR1 gene is capable of encoding a protein which confers resistance to a root-knot nematode to a plant of the Solanaceae family. In particular, the MeR1 gene is capable of encoding a protein which confers resistance to M. enterolobii to a Solanum lycopersicum plant. The genomic sequence which may comprise SEQ ID NO: 1 leads to 8 different isoforms which may comprise the coding sequence (CDS sequence) selected from the group consisting of SEQ ID Nos. 2, 3, 4, 5, 6, 7, 8 and 9. The isoforms are different splice variants of the gene. After splicing the genomic sequence which may comprise SEQ ID NO: 1 of the MeR1 gene of the invention leads to the 8 different isoforms which may comprise the coding sequence (CDS sequence) of the MeR1 gene of the invention selected from the group consisting of SEQ ID Nos. 2, 3, 4, 5, 6, 7, 8 and 9. These CDS sequences encode a protein which may comprise the amino acid sequence selected from the group consisting of SEQ ID Nos 10, 11, 12, 13, 14, 15, 16 and 17; SEQ ID NO: 2 encodes the MeR1 protein with an amino acid sequence which may comprise SEQ ID NO: 10, SEQ ID NO: 3 encodes the MeR1 protein with an amino acid sequence which may comprise SEQ ID NO: 11, SEQ ID NO: 4 encodes the MeR1 protein with an amino acid sequence which may comprise SEQ ID NO: 12, SEQ ID NO: 5 encodes the MeR1 protein with an amino acid sequence which may comprise SEQ ID NO: 13, SEQ ID NO: 6 encodes the MeR1 protein with an amino acid sequence which may comprise SEQ ID NO: 14, SEQ ID NO: 7 encodes the MeR1 protein with an amino acid sequence which may comprise SEQ ID NO: 15, SEQ ID NO: 8 encodes the MeR1 protein with an amino acid sequence which may comprise SEQ ID NO: 16 and SEQ ID NO: 9 encodes the MeR1 protein with an amino acid sequence which may comprise SEQ ID NO: 17.

During the research leading to the invention it was found that the MeR1 gene of the invention segregates together with another resistance gene called NRL (Nucleotide-binding domain and leucine-rich repeat) required for cell death 6 NRC6. The genome of a Solanum lycopersicum plant can comprise one or more NRC6 gene(s) herein called NRC6-2a, NRC6-2b etc. The nucleic acid molecules NRC6-2a, NRC6-2c, NRC6-2b, NRC6-3 and NRC6-1a are alleles of the NRC6 gene which encode a protein which when present with the MeR1 gene of the invention contributes to resistance to a root-knot nematode to a plant of the Solanaceae family. The nucleic acid molecule NRC6-2a which may comprise the genomic sequence according to SEQ ID NO: 18, encodes a protein which when present with the MeR1 gene of the invention contributes to resistance to a root-knot nematode within a plant of the Solanaceae family. The CDS sequence of the NRC6-2a allele of the invention may comprise SEQ ID NO: 19 and encodes the NRC6 protein with the amino acid sequence which may comprise SEQ ID NO: 20. The nucleic acid molecule NRC6-2c which may comprise the genomic sequence according to SEQ ID NO: 21 encodes a protein which when present with the MeR1 gene of the invention contributes to resistance to a root-knot nematode to a plant of the Solanaceae family. The CDS sequence of the NRC6-2c allele of the invention may comprise SEQ ID NO: 22 and encodes the NRC6 protein with the amino acid sequence which may comprise SEQ ID NO: 23. The nucleic acid molecule NRC6-2b which may comprise the CDS sequence according to SEQ ID NO: 24 encodes an NRC6 protein with the amino acid sequence which may comprise SEQ ID NO: 25, which when present with the MeR1 gene of the invention contributes to resistance to a root-knot nematode to a plant of the Solanaceae family. The nucleic acid molecule NRC6-3 which may comprise the CDS sequence according to SEQ ID NO: 26 encodes an NRC6 protein with the amino acid sequence which may comprise SEQ ID NO: 27, which when present with the MeR1 gene of the invention contributes to resistance to a root-knot nematode to a plant of the Solanaceae family. The nucleic acid molecule NRC6-1a which may comprise the CDS sequence according to SEQ ID NO: 28 encodes an NRC6 protein with the amino acid sequence which may comprise SEQ ID NO: 29, which when present with the MeR1 gene of the invention contributes to resistance to a root-knot nematode to a plant of the Solanaceae family.

The invention further relates to a nucleic acid molecule encoding a NRC6 protein, which contributes to resistance to root-knot nematode, in particular to M. enterolobii, when present in a plant of the Solanaceae family, in particular in a Solanum lycopersicum plant, together with the nucleic acid molecule encoding an MeR1 protein as defined above, wherein said nucleic acid molecule has a nucleotide sequence which is:
a) a nucleotide sequence which may comprise a coding sequence according to SEQ ID NO: 19, or a coding sequence having 98%, preferably 99% sequence identity with the sequence according to SEQ ID NO: 19; or
b) a nucleotide sequence encoding a protein having an amino acid sequence according to SEQ ID NO: 20, or a protein having an amino acid sequence which has 98%, preferably 99% sequence identity with the amino acid sequence according to SEQ ID NO: 20.

In one embodiment the invention relates to a nucleic acid molecule encoding a NRC6 protein, which contributes to resistance to root-knot nematode, in particular to M. enterolobii, when present in a plant of the Solanaceae family, in particular in a Solanum lycopersicum plant, together with the nucleic acid molecule encoding an MeR1 protein as defined above, wherein said nucleic acid molecule has a nucleotide sequence which is:
a) a nucleotide sequence which may comprise a coding sequence according to SEQ ID NO: 22, or a coding sequence having 98%, preferably 99% sequence identity with the sequence according to SEQ ID NO: 22; or
b) a nucleotide sequence encoding a protein having an amino acid sequence according to SEQ ID NO: 23, or a protein having an amino acid sequence which has 98%, preferably 99% sequence identity with the amino acid sequence according to SEQ ID NO: 23.

In a further embodiment, the invention relates to a nucleic acid molecule encoding a NRC6 protein, which contributes to resistance to root-knot nematode, in particular to M. enterolobii, when present in a plant of the Solanaceae family, in particular in a Solanum lycopersicum plant, together with the nucleic acid molecule encoding an MeR1 protein as defined above, wherein said nucleic acid molecule has a nucleotide sequence which is:
a) a nucleotide sequence which may comprise a coding sequence according to SEQ ID NO: 24, or a coding sequence having 98%, preferably 99% sequence identity with the sequence according to SEQ ID NO: 24; or
b) a nucleotide sequence encoding a protein having an amino acid sequence according to SEQ ID NO: 25, or a protein having an amino acid sequence which has 98%, preferably 99% sequence identity with the amino acid sequence according to SEQ ID NO: 25.

In a further embodiment, the invention relates to a nucleic acid molecule encoding a NRC6 protein, which contributes to resistance to root-knot nematode, in particular to M. enterolobii, when present in a plant of the Solanaceae family, in particular in a Solanum lycopersicum plant, together with the nucleic acid molecule encoding an MeR1 protein as defined above, wherein said nucleic acid molecule has a nucleotide sequence which is:
a) a nucleotide sequence which may comprise a coding sequence according to SEQ ID NO: 26, or a coding sequence having 98%, preferably 99% sequence identity with the sequence according to SEQ ID NO: 26; or
b) a nucleotide sequence encoding a protein having an amino acid sequence according to SEQ ID NO: 27, or a protein having an amino acid sequence which has 98%, preferably 99% sequence identity with the amino acid sequence according to SEQ ID NO: 27.

In a further embodiment, the invention relates to a nucleic acid molecule encoding a NRC6 protein, which contributes to resistance to root-knot nematode, in particular to M. enterolobii, when present in a plant of the Solanaceae family, in particular in a Solanum lycopersicum plant, together with the nucleic acid molecule encoding an MeR1 protein as defined above, wherein said nucleic acid molecule has a nucleotide sequence which is:
a) a nucleotide sequence which may comprise a coding sequence according to SEQ ID NO: 28, or a coding sequence having 98%, preferably 99% sequence identity with the sequence according to SEQ ID NO: 28; or
b) a nucleotide sequence encoding a protein having an amino acid sequence according to SEQ ID NO: 29, or a protein having an amino acid sequence which has 98%, preferably 99% sequence identity with the amino acid sequence according to SEQ ID NO: 29.

The nucleic acid molecules MeR1 and NRC6 of the invention each encode an NBS-LRR protein, which is also called NLR protein, and may comprise a nucleotide binding site and a leucine rich repeat domain. In recent research, it was shown that NBS-LRR proteins in the plant often communicate with each other to induce an immunity response to a pathogen, therefore in the plant genome so called “sensor” NBS-LRR proteins exist which detect the pathogen and need a “helper” NBS-LRR to induce the immune response after binding of the pathogen effector to the sensor NLR. The genomes of the Solanaceae plants comprise numerous NBS-LRR proteins and the network is very complex. It is therefore difficult to predict if an NBS-LRR protein is a helper or a sensor protein and which NBS-LRR helper protein interacts with which NBS-LRR sensor protein(s). Recently, it has been discovered by Adachi et al. ((2019) An N-terminal motif in NLR immune receptors is functionally conserved across distantly related plant species; Elife. 27; 8) that the so-called helper NLR proteins comprise a specific motif: the MADA-motif (SEQ ID NO:30). When analyzing the sequences of NRC6 it was found that the encoded protein may comprise the MADA-motif and can therefore be characterized as being a helper NLR protein. The MeR1 protein does not comprise the motif and can therefore be considered as a candidate for the sensor NLR protein which is able to communicate with a helper NLR protein.

The invention also relates to a plant of the Solanaceae family, in particular a Solanum lycopersicum plant, which may comprise the nucleic acid molecule encoding an MeR1 protein, which confers resistance to root-knot nematode, in particular to M. enterolobii, when present in a plant of the Solanaceae family, wherein said nucleic acid molecule has a nucleotide sequence which is:
a) a nucleotide sequence which may comprise a coding sequence according to SEQ ID NO: 2, 3, 4, 5, 6, 7, 8 or 9, or a coding sequence which has in order of increased preference 90%, 91%, 92%, 93%, 94%, 95%, 96%, 97%, 98%, 99% sequence identity with the sequence according to SEQ ID NO: 2, 3, 4, 5, 6, 7, 8 or 9; or
b) a nucleotide sequence encoding a protein having an amino acid sequence according to SEQ ID NO: 10, 11, 12, 13, 14, 15, 16 or 17, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 10, 11, 12, 13, 14, 15, 16 or 17;
and optionally at least one nucleic acid molecule encoding an NRC6-2a protein, wherein said nucleic acid molecule has a nucleotide sequence which is:
a) a nucleotide sequence which may comprise a coding sequence according to SEQ ID NO: 19, or a coding sequence having 98%, preferably 99% sequence identity with the sequence according to SEQ ID NO: 19; or
b) a nucleotide sequence encoding an NRC6-2a protein having an amino acid sequence according to SEQ ID NO: 20, or a protein having an amino acid sequence which has 98%, preferably 99% sequence identity with the amino acid sequence according to SEQ ID NO: 20;
and/or a nucleic acid molecule encoding an NRC6-2c protein, wherein said nucleic acid molecule has a nucleotide sequence which is:
a) a nucleotide sequence which may comprise a coding sequence according to SEQ ID NO: 22, or a coding sequence having 98%, preferably 99% sequence identity with the sequence according to SEQ ID NO: 22; or
b) a nucleotide sequence encoding an NRC6-2c protein having an amino acid sequence according to SEQ ID NO: 23, or a protein having an amino acid sequence which has 98%, preferably 99% sequence identity with the amino acid sequence according to SEQ ID NO: 23;
and/or a nucleic acid molecule encoding an NRC6-2b protein, wherein said nucleic acid molecule has a nucleotide sequence which is:
a) a nucleotide sequence which may comprise a coding sequence according to SEQ ID NO: 24, or a coding sequence having 98%, preferably 99% sequence identity with the sequence according to SEQ ID NO: 24; or
b) a nucleotide sequence encoding an NRC6-2b protein having an amino acid sequence according to SEQ ID NO: 25, or a protein having an amino acid sequence which has 98%, preferably 99% sequence identity with the amino acid sequence according to SEQ ID NO: 25;
and/or a nucleic acid molecule encoding an NRC6-3 protein, wherein said nucleic acid molecule has a nucleotide sequence which is:
a) a nucleotide sequence which may comprise a coding sequence according to SEQ ID NO: 26, or a coding sequence having 98%, preferably 99% sequence identity with the sequence according to SEQ ID NO: 26; or
b) a nucleotide sequence encoding an NRC6-3 protein having an amino acid sequence according to SEQ ID NO: 27, or a protein having an amino acid sequence which has 98%, preferably 99% sequence identity with the amino acid sequence according to SEQ ID NO: 27;
and/or a nucleic acid molecule encoding an NRC6-1a protein, wherein said nucleic acid molecule has a nucleotide sequence which is:
a) a nucleotide sequence which may comprise a coding sequence according to SEQ ID NO: 28, or a coding sequence having 98%, preferably 99% sequence identity with the sequence according to SEQ ID NO: 28; or
b) a nucleotide sequence encoding an NRC6-1a protein having an amino acid sequence according to SEQ ID NO: 29, or a protein having an amino acid sequence which has 98%, preferably 99% sequence identity with the amino acid sequence according to SEQ ID NO: 29.

In particular, the invention relates to a plant of the Solanaceae family, in particular a Solanum lycopersicum plant, which may comprise a nucleic acid molecule encoding an MeR1 protein, which confers resistance to root-knot nematode, in particular to M. enterolobii, when present in a plant of the Solanaceae family, wherein said nucleic acid molecule has a nucleotide sequence which is:
a) a nucleotide sequence which may comprise a coding sequence according to SEQ ID NO: 2, 3, 4, 5, 6, 7, 8 or 9, or a coding sequence which has in order of increased preference 90%, 91%, 92%, 93%, 94%, 95%, 96%, 97%, 98%, 99% sequence identity with the sequence according to SEQ ID NO: 2, 3, 4, 5, 6, 7, 8 or 9; or
b) a nucleotide sequence encoding a protein having an amino acid sequence according to SEQ ID NO: 10, 11, 12, 13, 14, 15, 16 or 17, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 10, 11, 12, 13, 14, 15, 16 or 17;
and a nucleic acid molecule encoding an NRC6-2a protein, wherein said nucleic acid molecule has a nucleotide sequence which is:
a) a nucleotide sequence which may comprise a coding sequence according to SEQ ID NO: 19, or a coding sequence having 98%, preferably 99% sequence identity with the sequence according to SEQ ID NO: 19; or
b) a nucleotide sequence encoding an NRC6-2a protein having an amino acid sequence according to SEQ ID NO: 20, or a protein having an amino acid sequence which has 98%, preferably 99% sequence identity with the amino acid sequence according to SEQ ID NO: 20;
and a nucleic acid molecule encoding an NRC6-2c protein, wherein said nucleic acid molecule has a nucleotide sequence which is:
a) a nucleotide sequence which may comprise a coding sequence according to SEQ ID NO: 22, or a coding sequence having 98%, preferably 99% sequence identity with the sequence according to SEQ ID NO: 22; or
b) a nucleotide sequence encoding an NRC6-2c protein having an amino acid sequence according to SEQ ID NO: 23, or a protein having an amino acid sequence which has 98%, preferably 99% sequence identity with the amino acid sequence according to SEQ ID NO: 23.

The invention also relates to a plant of the Solanaceae family, in particular a Solanum lycopersicum plant, which may comprise the nucleic acid molecule encoding an MeR1 protein as described herein, which confers resistance to root-knot nematode, in particular to M. enterolobii, when present in a plant of the Solanaceae family, and one NRC6 gene as described herein and selected from the group consisting of NRC6-2a, NRC6-2c, NRC6-2b, NRC6-3 and NRC6-1a.

The invention also relates to a plant of the Solanaceae family, in particular a Solanum lycopersicum plant, which may comprise the nucleic acid molecule encoding an MeR1 protein as described herein, which confers resistance to root-knot nematode, in particular to M. enterolobii, when present in a plant of the Solanaceae family, and two NRC6 genes as described herein and selected from the group consisting of NRC6-2a, NRC6-2c, NRC6-2b, NRC6-3 and NRC6-1a.

The invention also relates to a plant of the Solanaceae family, in particular a Solanum lycopersicum plant, which may comprise the nucleic acid molecule encoding an MeR1 protein as described herein, which confers resistance to root-knot nematode, in particular to M. enterolobii, when present in a plant of the Solanaceae family, and three NRC6 genes as described herein and selected from the group consisting of NRC6-2a, NRC6-2c, NRC6-2b, NRC6-3 and NRC6-1a.

The invention also relates to a plant of the Solanaceae family, in particular a Solanum lycopersicum plant, which may comprise the nucleic acid molecule encoding an MeR1 protein as described herein, which confers resistance to root-knot nematode, in particular to M. enterolobii, when present in a plant of the Solanaceae family, and four NRC6 genes as described herein and selected from the group consisting of NRC6-2a, NRC6-2c, NRC6-2b, NRC6-3 and NRC6-1a.

The invention also relates to a plant of the Solanaceae family, in particular a Solanum lycopersicum plant, which may comprise the nucleic acid molecule encoding an MeR1 protein as described herein, which confers resistance to root-knot nematode, in particular to M. enterolobii, when present in a plant of the Solanaceae family, and five NRC6 genes as described herein and selected from the group consisting of NRC6-2a, NRC6-2c, NRC6-2b, NRC6-3 and NRC6-1a.

A plant of the Solanaceae family, can be a plant of the species Solanum lycopersicum, Capsicum annuum, Solanum melongena, Solanum tuberosum, or Nicotiana tabacum.

Preferably, the invention also relates to a Solanum lycopersicum plant which may comprise one or more of the NRC6 alleles as defined above which allele(s) contribute(s) to resistance to M. enterolobii when present in said plant together with the MeR1 nucleic acid molecule of the invention. In this application the term tomato and Solanum lycopersicum are used interchangeably.

The phenotype of the invention inherits in a dominant manner. As used herein, dominant means that when the nucleic acid molecule(s) of the invention is/are heterozygously present in a plant, the resistance level to root-knot nematode of the plant is the same as if the nucleic acid molecule(s) of the invention would be homozygously present in a plant. A Solanum lycopersicum plant that has the nucleic acid molecules of the invention homozygously can be grown from seed deposited as NCIMB 43515. Such plants show M. enterolobii resistance.

A Solanum lycopersicum plant which may comprise the MeR1 gene of the invention and two NRC6 alleles (NRC6-2a and NRC6-2c) can be grown from a seed, a representative sample of which was deposited with the NCIMB under deposit number NCIMB 43515.

The presence of resistance to a root-knot nematode can be determined through a bioassay as described in Example 1. Determining resistance of a Solanum lycopersicum plant to M. enterolobii is done by counting the egg masses. The Solanum lycopersicum plants to be tested are sown in trays in a greenhouse, after 2 weeks the plants are transferred to 660 cc pots filled with a sandy soil mixture. As a susceptible control plant Dometica RZ is introduced in the test and as a positive control a plant grown from for a seed of the deposit NCIMB 43515 is introduced in the test. Next to the roots, holes are made with a 1 ml pipet tip and filled with 1 ml suspension which may comprise 500 of the second stage juveniles (J2) M. enterolobii inoculum per plant. After inoculation, the plants are grown in a growth chamber (23° C., 14 h. light) and watered three times a week. Six weeks after inoculation the plants are removed from the soil and the roots are washed with tap water. The egg masses are counted and the scoring is done according to the symptoms as presented in Table 2. An M. enterolobii resistant tomato plant has a score of 0, 1 or 2, preferably 0 or 1, when scoring according to Table 2 is used.

A plant of the present invention is optionally a cultivated plant having improved agronomic characteristics that make it suitable for commercial cultivation. This is thus an agronomically elite plant. The invention also relates to a fruit of a plant of the Solanaceae family harvested from a plant of the invention, wherein the fruit may comprise the nucleic acid molecule(s) of the invention in its genome which leads to resistance to a root-knot nematode, in particular to M. enterolobii, in the plant. This fruit is also referred to herein as ‘the fruit of the invention’. Preferably, the fruit is a tomato fruit of a Solanum lycopersicum plant.

The present invention relates to a rootstock or scion of a plant of the Solanaceae family which may comprise the nucleic acid molecule(s) of the invention. Said rootstock or scion can be used in a grafting process to develop a grafted plant of the Solanaceae family which shows the phenotype of the invention and which grafted plant is also part of the invention. In one embodiment, the invention further relates to a rootstock which results from an interspecific cross between plants belonging to different species of the Solanaceae family wherein one parent plant of the interspecific cross is preferably a plant of the species Solanum lycopersicum which may comprise the nucleic acid molecule(s) of the invention and the other parent is selected from the group consisting of Solanum arcanum, Solanum cheesmaniae, Solanum chilense, Solanum chmielewskii, Solanum corneliomulleri, Solanum galapagense, Solanum habrochaites, Solanum huaylasense, Solanum juglandifolium, Solanum lycopersicum cerasiforme, Solanum lycopersicoides, Solanum neorickii, Solanum ochrantum, Solanum pennellii, Solanum peruvianum, Solanum pimpinellifolium and Solanum sitiens.

The present invention further relates to the use of the plant of the invention in a grafting process, wherein the plant of the invention can either be used as a scion or preferably as a rootstock in the grafting process. The other plant used in the grafting process can be any plant of the Solanaceae family and preferably belongs to the Solanum genus.

The present invention relates to a method for producing plant of the Solanaceae family which is resistant to a root-knot nematode, in particular to M. enterolobii, which may comprise introducing the nucleic acid molecule(s) of the invention into the plant.

The nucleic acid molecule(s) of the invention can be introduced from another plant, which may comprise the nucleic acid molecule(s), through commonly used breeding techniques, such as crossing and selection, when the plants are sexually compatible. Such introduction can be from a plant of the same species, that usually can be crossed easily, or from a plant of a related species. Difficulties in crossing can be overcome through techniques known in the art such as embryo rescue, or cis-genesis. Suitably methods for the identification of the nucleic acid molecule are used to follow the incorporation of the nucleic acid molecule into another plant.

Alternatively, the nucleic acid molecule(s) of the invention can be transferred or introduced from another, sexually incompatible, plant, for example by using a transgenic approach. Techniques that can suitably be used comprise general plant transformation techniques known to the skilled person, such as the use of an Agrobacterium-mediated transformation method. Genome editing methods such as the use of a CRISPR/Cas system might also be employed to obtain a plant of the invention. The CRISPR/Cas system may, for example, comprise a Cas9, Cpf1, Cms1, MAD7, C2c2, CasX and/or CasY protein.

The invention further relates to a plant of the invention which may comprise the nucleic acid molecule(s) of the invention leading to resistance to a root-knot nematode, in particular to M. enterolobii, either homozygously or heterozygously, which plant is a plant of an inbred line, a hybrid, a doubled haploid, or a plant of a segregating population. Preferably, the plant of the invention is a non-transgenic plant.

The invention also relates to a seed of a plant of the Solanaceae family which may comprise the nucleic acid molecule(s) of the invention, wherein the plant of the Solanaceae family grown from the seed is a plant of the invention that is resistant to a root-knot nematode, in particular to M. enterolobii. Preferably, the plant is a Solanum lycopersicum plant. The invention also relates to seed produced by a plant of the invention wherein the seed harbors the nucleic acid molecule(s) of the invention, and as such, a plant grown from said seed is a plant of the invention.

Moreover, the invention also relates to a food product or a processed food product which may comprise the fruit of the invention or part thereof. The food product may have undergone one or more processing steps. Such a processing step might comprise but is not limited to any one of the following treatments or combinations thereof: peeling, cutting, washing, juicing, cooking, cooling. A processed food product may also be a salad mixture which may comprise the fruit of the invention. The fruit is a fruit of a plant of a species of the Solanaceae family, in particular pepper, eggplant, potato, tomato. Preferably, the fruit is a tomato fruit of the invention. The processed form that is obtained is also part of this invention.

The invention also relates to propagation material suitable for producing a plant of the invention, wherein the propagation material is suitable for sexual reproduction, and is in particular selected from a microspore, pollen, an ovary, an ovule, an embryo sac, and an egg cell; or is suitable for vegetative reproduction, and is in particular selected from a cutting, a root, a stem, a cell, a protoplast; or is suitable for tissue culture of regenerable cells, and is in particular selected from a leaf, pollen, an embryo, a cotyledon, a hypocotyl, a meristematic cell, a root, a root tip, an anther, a flower, a seed, a rootstock and a stem; wherein the plant produced from the propagation material may comprise the nucleic acid molecule(s) of the invention that confers resistance to a root-knot nematode, preferably resistance to M. enterolobii. A plant of the invention may be used as a source of the propagation material.

The invention further relates to a cell which may comprise the nucleic acid molecule(s) of the invention as defined herein. A cell of the invention can be obtained from, or be present in, a plant of the invention. Such a cell may either be in isolated form, or is a part of a complete plant, or from a part thereof, and still constitutes a cell of the invention because such a cell may comprise the nucleic acid molecule(s) that encode(s) the protein(s) that confer(s) resistance to a root-knot nematode, in particular to M. enterolobii, to a cultivated plant of the invention. Each cell of a plant of the invention carries the nucleic acid molecule(s) encoding the protein(s) conferring resistance to a root-knot nematode, in particular to M. enterolobii, to a plant. A cell of the invention may also be a regenerable cell that can regenerate into a new plant of the invention.

The invention further relates to plant tissue of a plant of the invention, which may comprise the nucleic acid molecule(s) of the invention. The tissue can be undifferentiated tissue or already differentiated tissue. Undifferentiated tissue is for example a stem tip, an anther, a petal, pollen, and can be used in micropropagation to obtain new plantlets that are grown into new plants of the invention. The tissue can also be grown from a cell of the invention.

The invention further relates to tissue culture of a plant of the invention, which is also propagation material and which may comprise the nucleic acid molecule(s) of the invention in its genome. The tissue culture may comprise regenerable cells. Such tissue culture can be selected or derived from any part of the plant, in particular from a leaf, pollen, an embryo, a cotyledon, a hypocotyl, a meristematic cell, a root, a root tip, an anther, a flower, a seed, a stem or a rootstock. The tissue culture can be regenerated into a plant of the invention which may comprise the nucleic acid molecule(s) of the invention, wherein the regenerated plant expresses resistance to a root-knot nematode, in particular to M. enterolobii, and is also part of the invention.

The invention additionally relates to the use of a plant of the invention in plant breeding. The invention thus also relates to a breeding method for the development of a cultivated plant of the invention that is resistant to a root-knot nematode, in particular to M. enterolobii, wherein a plant which may comprise the nucleic acid molecule(s) of the invention for conferring said resistance to another plant is used. Preferably, the plant of the invention used in plant breeding is a Solanum lycopersicum plant. Seed of a Solanum lycopersicum being representative for a plant that can be used in plant breeding to develop another plant with resistance to a root-knot nematode, in particular to M. enterolobii, was deposited with the NCIMB under accession numbers NCIMB 43515.

The invention also concerns the use of the nucleic acid molecule(s) of the invention for the development of a plant that has resistance to a root-knot nematode. Preferably, the developed plant is a Solanum lycopersicum plant that has resistance to M. enterolobii.

The invention also relates to a method of testing a plant for the presence in its genome of the nucleic acid molecule(s) of the invention, which may comprise detecting the presence of the nucleic acid molecule(s) of the invention in the genome of a plant.

The method of testing a plant for the presence in its genome of the nucleic acid molecule(s) of the invention encoding the protein(s) conferring resistance to a root-knot nematode, in particular to M. enterolobii, optionally further may comprise selecting a plant that may comprise said nucleic acid molecule(s) as a plant resistant to a root-knot nematode.

The present invention relates to a method for selecting a root-knot nematode resistant plant of the Solanaceae family, in particular a Solanum lycopersicum plant, wherein the selection may comprise determining the presence of a nucleic acid molecule(s) of the invention in the genome of a plant of the Solanaceae family, and selecting the plant if it may comprise the nucleic acid molecule(s) of the invention. A plant in which the nucleic acid molecule(s) is identified is subsequently selected as a plant resistant to a root-knot nematode. In one embodiment, the root-knot nematode is M. enterolobii which can be detected by performing a bio-assay as described in Example 1. The selected plant obtained by such method is also a part of this invention. The invention further relates to a method for selecting a plant of the Solanaceae family, in particular a Solanum lycopersicum plant, carrying the nucleic acid molecule(s) of the invention, which may comprise determining the presence of the nucleic acid molecule(s) of the invention by determining its genomic nucleotide sequence or a functional part thereof in the genome of a plant, wherein said sequence has in order of increased preference 80%, 81%, 82%, 83%, 84%, 85%, 86%, 87%, 88%, 89%, 90%, 91%, 92%, 93%, 94%, 95%, 96%, 97%, 98%, 99%, 100% sequence identity to a SEQ ID NO: selected from the group consisting of SEQ ID NO:1, 18 and 21.

The invention further relates to a method for selecting a plant of the Solanaceae family, in particular a Solanum lycopersicum plant, carrying the nucleic acid molecule(s) of the invention, which may comprise determining the presence of the nucleic acid molecule(s) of the invention by determining its coding sequence or a part thereof in the plant, wherein said sequence has in order of increased preference 90%, 91%, 92%, 93%, 94%, 95%, 96%, 97%, 98%, 99%, 100% sequence identity to a SEQ ID NO: selected from the group consisting of SEQ ID NO: 2, 3, 4, 5, 6, 7, 8, 9, 19, 22, 24, 26 and 28.

The invention further relates to a method for selecting a plant of the Solanaceae, in particular a Solanum lycopersicum plant, family carrying the nucleic acid molecule(s) of the invention, which may comprise determining the presence of the nucleic acid molecule(s) of the invention by determining the amino acid sequence of the encoded protein(s), wherein said sequence has in order of increased preference 85%, 86%, 87%, 88%, 89%, 90%, 91%, 92%, 93%, 94%, 95%, 96%, 97%, 98%, 99%, 100% sequence identity to a SEQ ID NO: selected from the group consisting of SEQ ID NO: 10, 11, 12, 13, 14, 14, 16, 17, 20, 23, 25, 27 and 29.

The invention also relates to a method for the production of a plant, which is resistant to a root-knot nematode, in particular to M. enterolobii, said method which may comprise:
crossing a plant of the invention, which may comprise the nucleic acid molecule(s) of the invention, with another plant;
optionally performing one or more rounds of selfing and/or crossing of the plant resulting from the cross to obtain a further generation population;
selecting from the plants resulting from the cross, or from the further generation population, a plant that may comprise the nucleic acid molecule(s) of the invention, which plant is resistant against a root-knot nematode, in particular against M. enterolobii.

Selection of a plant which may comprise the nucleic acid molecule(s) of the invention is suitably done by comparing the sequences to any one of the sequences selected from the group consisting of SEQ ID Nos 1, 2, 3, 4, 5, 6, 7, 8, 9, 18, 19, 21, 22, 24, 26 and 28. The plant can alternatively, or in addition, be phenotypically selected for having resistance to a root-knot nematode, in particular by performing a bio-assay for M. enterolobii resistance.

In one embodiment of the invention, the plant of the invention used in the method for the production of a plant, which is resistant against a root-knot nematode, is a Solanum lycopersicum plant. In a further embodiment of the invention, the plant of the invention used in the method for the production of a Solanum lycopersicum plant which is resistant against M. enterolobii, is a plant grown from seed deposited under NCIMB accession number NCIMB 43515, or a progeny plant thereof which is a direct or further descendant through crossing a plant grown from the deposited seed with itself or with another plant for one or more subsequent generations.

The invention additionally provides for a method of introducing another desired trait into a plant which may comprise resistance to a root-knot nematode, in particular to M. enterolobii, which may comprise:
crossing a plant of the invention which may comprise the nucleic acid molecule(s) of the invention with a second plant that may comprise the other desired trait to produce F1 progeny;
optionally selecting in the F1 for a plant that may comprise resistance to a root-knot nematode and the other desired trait;
crossing the optionally selected F1 progeny with either parent, to produce backcross progeny;
selecting backcross progeny which may comprise resistance to a root-knot nematode and the other desired trait; and
optionally repeating steps c) and d) one or more times in succession to produce selected fourth or higher backcross progeny that may comprise the other desired trait and has resistance to a root-knot-nematode. Backcrossing is optionally done until the backcross progeny is stable and can be used as a parent line, which can be reached after up to 10 backcrosses.

In one embodiment of the invention, the plant of the invention used in the method of introducing another desired trait into a plant which may comprise resistance to a root-knot nematode is a Solanum lycopersicum plant which may comprise resistance to M. enterolobii. In a further embodiment of the invention, the Solanum lycopersicum plant of the invention used in the method of introducing another desired trait into a plant which may comprise resistance to M. enterolobii is a plant grown from seed deposited under NCIMB accession number NCIMB 43515, or a progeny plant thereof which is a direct or further descendant through crossing a plant grown from the deposited seed with itself or with another plant for one or more subsequent generations.

Optionally, selfing steps are performed after any of the crossing or backcrossing steps. Selection of a plant which may comprise the nucleic acid molecule(s) of the invention that leads to resistance to a root-knot nematode and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method and the fruit obtained therefrom.

The invention further relates to a method for the production of a plant which may comprise the nucleic acid molecule(s) of the invention, wherein the presence of said nucleic acid molecule(s) leads to resistance to a root-knot nematode, in particular to M. enterolobii, by using vegetative reproduction, by using tissue culture of the plant material that may comprise the nucleic acid molecule(s) of the invention or by using a doubled haploid generation technique to generate a doubled haploid line and wherein the method may comprise growing a seed which may comprise said nucleic acid molecule into the said plant.

The invention further relates to a method for seed production which may comprise growing a plant from a seed of the invention, allowing the plant to produce a fruit with seed, harvesting the fruit, and extracting the seed. Production of the seed is suitably done by crossing with itself or with another plant that is optionally also a plant of the invention. The seed that is so produced has the capability to grow into a plant that may comprise the nucleic acid molecule(s) of the invention and is resistant to a root-knot nematode, in particular to M. enterolobii.

The invention further relates to hybrid seed and to a method for producing said hybrid seed, which may comprise crossing a first parent plant with a second parent plant and harvesting the resultant hybrid seed, wherein the first parent plant and/or the second parent plant is a plant of the invention which may comprise the nucleic acid molecule(s) of the invention. The resulting hybrid plant that can be grown from the hybrid seed, which may comprise said nucleic acid molecule(s), which hybrid plant has resistance to a root-knot nematode, in particular to M. enterolobii, is also a plant of the invention.

The parent that provides the resistance to a root-knot nematode, in particular to M. enterolobii, can be a plant grown directly from the deposited seed. The parent can also be a progeny plant from the deposited seed which is a direct or further descendant obtained by crossing with itself or with another plant one or more times, or a progeny plant from seed that is identified to have obtained the nucleic acid molecule(s) of the invention and thereby the resistance to a root-knot nematode, in particular to M. enterolobii, by other means.

Introgression of the nucleic acid molecule(s) of the invention as used herein means introduction of the nucleic acid molecule(s) of the invention from a donor plant which may comprise said nucleic acid molecule(s) into a recipient plant not carrying said nucleic acid molecule(s) by standard breeding techniques, wherein selection for plants which may comprise the nucleic acid molecule(s) of the invention can be performed phenotypically by means of observation of the resistance to a root-knot nematode, in particular to M. enterolobii, or selection can be performed by comparing the sequence to the any one of the sequences selected from the group consisting of SEQ ID Nos 1, 2, 3, 4, 5, 6, 7, 8, 9, 18, 19, 21, 22, 24, 26 and 26, as described herein.

The nucleic acid molecule(s) of the invention is/are introgressed into a plant of the Solanaceae family from another plant of the Solanaceae family. In one embodiment, the nucleic acid molecule(s) of the invention is/are introgressed into Solanum lycopersicum from a plant of the species Solanum pimpinellifolium. In another embodiment, the nucleic acid molecule(s) is/are introgressed from a Solanum lycopersicum plant which may comprise the nucleic acid molecule(s) of the invention into a Solanum lycopersicum plant lacking the nucleic acid molecule(s) of the invention. In a further embodiment of the invention, the nucleic acid molecule(s) is/are introgressed from a Solanum lycopersicum plant or a Solanum pimpinellifolium, which may comprise the nucleic acid molecule(s) of the invention, into a plant lacking the nucleic acid molecule(s) of the invention and wherein the plant is selected from the group consisting of Solanum arcanum, Solanum cheesmaniae, Solanum chilense, Solanum chmielewskii, Solanum corneliomulleri, Solanum galapagense, Solanum habrochaites, Solanum huaylasense, Solanum juglandifolium, Solanum lycopersicum cerasiforme, Solanum lycopersicoides, Solanum neorickii, Solanum ochrantum, Solanum pennellii, Solanum peruvianum, Solanum pimpinellifolium and Solanum sitiens. In particular the MeR1 gene of the invention is introgressed into a plant of the Solanaceae family from another plant of the Solanaceae family. In one particular embodiment, the MeR1 gene of the invention is introgressed into Solanum lycopersicum from a plant of the species Solanum pimpinellifolium.

As used herein, the “phenotype of the invention”, “the resistance of the invention” is resistance to a root-knot nematode. Preferably, the resistance to a root-knot nematode is resistance to the root-knot nematode Meloidogyne enterolobii (Yang & Eisenback, 1983). A synonym of M. enterolobii is Meloidogyne mayaguensis or M. mayaguensis.

As used herein the term “nucleic acid molecule” is used to designate the nucleotide sequence of a gene or of an allele of the gene which is a specific sequence of a gene linked to as a specific phenotype, i.e. the phenotype of the invention. Optionally, the nucleic acid molecule is isolated. As used herein “MeR1 gene of the invention” or “MeR1 nucleic acid molecule of the invention” is used to designate the MeR1 gene, which leads to the phenotype of the invention. The invention relates to a gene or an allele, which encodes a functional protein leading to the resistance. More specifically, “MeR1 gene of the invention” or “MeR1 nucleic acid molecule of the invention” is used to designate a nucleic acid molecule encoding an MeR1 protein, which confers resistance to root-knot nematode, in particular to M. enterolobii, when present in a plant of the Solanaceae family, in particular in a Solanum lycopersicum plant, wherein said nucleic acid molecule has a nucleotide sequence which is:
a) a nucleotide sequence which may comprise a coding sequence according to SEQ ID NO: 2, 3, 4, 5, 6, 7, 8 or 9, or a coding sequence which has in order of increased preference 90%, 91%, 92%, 93%, 94%, 95%, 96%, 97%, 98%, 99% sequence identity with the sequence according to SEQ ID NO: 2, 3, 4, 5, 6, 7, 8 or 9; or
b) a nucleotide sequence encoding a protein having an amino acid sequence according to SEQ ID NO: 10, 11, 12, 13, 14, 15, 16 or 17, or a protein having an amino acid sequence which has in order of increased preference 85%, 86%, 87%, 88%, 89%, 90%, 91%, 92%, 93%, 94%, 95%, 96%, 97%, 98%, 99% sequence identity with the amino acid sequence according to SEQ ID NO: 10, 11, 12, 13, 14, 15, 16 or 17.

As used herein the term “NRC6 allele” is used to designate an allele of the NRC6 gene which contributes to root-knot nematode resistance, in particular to M. enterolobii, in a plant when present together with the MeR1 gene of the invention. More specifically, “NRC6 allele” is used to designate nucleic acid molecule encoding a protein, which contributes to resistance to root-knot nematode, in particular to M. enterolobii, when present in a plant of the Solanaceae family, in particular a Solanum lycopersicum, together with the MeR1 nucleic acid molecule of the invention, wherein said nucleic acid molecule has a nucleotide sequence which is:
a) a nucleotide sequence which may comprise a coding sequence according to SEQ ID NO: 19, 22, 24, 26 or 28 or a coding sequence having 98%, preferably 99% sequence identity with the sequence according to SEQ ID NO: 19, 22, 24, 26 or 28; or
b) a nucleotide sequence encoding a protein having an amino acid sequence according to SEQ ID NO: 20, 23, 25, 27 or 29 or a protein having an amino acid sequence which has 98%, preferably 99% sequence identity with the amino acid sequence according to SEQ ID NO: 20, 23, 25, 27 or 29.

In general the plants of the Solanum genus and in particular the Solanum lycopersicum plants comprise an NRC6 allele of the NRC6 gene. Examples of alleles are given herein. The presence of a NRC6 gene alone in a plant does not lead to M. enterolobii resistance. For having a plant of the Solanaceae family, in particular a Solanum lycopersicum, resistant to root-knot nematode, in particular to M. enterolobii, at least the MeR1 gene of the invention should be present in said plant and preferably in combination with a NRC6 gene.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment, whereby in the present case a sequence represents a gene or an allele that at least may comprise a start codon and a stop codon, or a complete protein encoded by such a gene or allele.

In the context of this invention “nucleic acid molecules of the invention” is used to designate the nucleic acid molecule defined as “MeR1 gene of the invention” as defined above and at least one nucleic acid molecule defined as “NRC6 allele” as defined above, which can be the NRC6-2a, NRC6-2c, NRC6-2b, NRC6-3 or NRC6-1a allele as defined herein or any combination thereof.

As used herein “nucleic acid molecule(s) of the invention” means the MeR1 gene of the invention or the MeR1 gene of the invention optionally in combination with at least one NRC6 allele which can be the NRC6-2a, NRC6-2c, NRC6-2b, NRC6-3 or NRC6-1a allele as defined herein or any combination thereof.

As used herein, the “plant of the invention” is a plant of the family of the Solanaceae family, which may comprise the nucleic acid molecule(s) of the invention and which is resistant to root-knot nematode, preferably the plant of the invention belongs to the genus Solanum, even more preferably the plant is a Solanum lycopersicum plant, which is resistant to M. enterolobii.

As used herein “progeny” is intended to mean the first and all further descendants from a cross with a plant of the invention, wherein a cross may comprise a cross with itself or a cross with another plant, and wherein a descendant that is determined to be progeny may comprise the nucleic acid molecule(s) of the invention as defined herein leading to resistance to a root-knot nematode, in particular to M. enterolobii. “Progeny” also encompasses a plant that carries the nucleic acid molecule(s) of the invention and is resistant to a root-knot nematode, in particular to M. enterolobii, and is obtained from another plant, or progeny of a plant, of the invention by vegetative propagation or another form of multiplication.

TABLE 1
Sequence information
The genomic sequences correspond to the mRNA with +2 kb upstream and downstream
from the start and the end.

| | |
|---|---|
| SEQ ID NO: | TTTCGCACTTCACTGAAACATTAAATTGGGCAACATTACTCTGT |
| 1: genomic | TATACTAATAATACAGATTTTAGGTGCCTTGATATCAAACATA |
| MeR1MeR1 | GAGTAAATTCAATTATTTTCGTGTTCATCACATTCCTCATCTAT |
| | TTTTCTCTTACTTATTTTCCTCCTTTTTAGCAGGAGAGTATCGAG |
| | GCATGGTGATGCATTTGCAGTCTCAGTTTTGATTACCTGTTCCA |
| | ATAAAGAAACTTGTTACAAGTATGTTTTGTTGATTCAAACATG |
| | CCAAGTTTGAAAACGAAGTTTTATATTAATGTTTTTGCGTTTTC |
| | TTTCAGTGCTGAATAATTTGTCTAATCTGTAGTTGATGTTTGGA |
| | TATCTATGTATTGTTCGATTGACACGAGTTGTTTATACTATCAT |
| | TTGTTGTTTATGAATATGTAAAACATATTTGATTTATGTTTGCA |
| | ACGTCAACGTGAACATTCAATTAGTCTAATCAAAGTCTAGATC |
| | TAAAATTGATTAATTTAAGTTCATAATTTATTTTGAATTATTAT |
| | CTAAAACCGAAGACTTTCTACTCGAAAATCGAAATCATAGCTG |
| | ACAGTCTCCATAAATATATTTGCAATTGAATAACATAATAAGC |
| | ATTTATCAGTTAATTTTATGGAGTTTCTGGATTTACGTGATATT |
| | CAAACATCTCCCGCGCATATTAATTATGTGAAGTAATGAAGTG |
| | TGTAACATAAGTCAAAAGGTGCATAAATAAAGAGTCAAGGGT |
| | AATAAGACTTTAGTTTACTTTAGGTGTGTCTCTGAAATTTCGAT |
| | TATAATCTACGATATATTGTGTCTTGTCCCAACATTTAAATAAA |
| | ATGAACAAATAATTTTAATTACCAAATGGATCCATAGTCTGTTT |
| | AGTTGACTAATGGAAAGATTTATAGGAAGGTGAGATTGTTTTA |
| | TCCCTGATTAGAGGTTTTGGTTCGAGTTTTGTGTATACGAAAAA |
| | ATTGTGTTGAAAGCGTCACTTATGAATAGGCCCTATAATGTAT |
| | GATTCGAATTTAGTCTGAGTTCTAATATGAATTTTGAACACATT |
| | GAAAAATTATATCCTTTGTTAAATTTCATGGTTTTAACATCTTG |
| | GAAGTAAAGAAAAAGGTAAAATATTTTTTTTTTCTATTTCATTT |
| | TTTTACATGTAATATTTGAATTCAAAGGCTGAGAGAGTAATAC |
| | CCCCCTTTTATTTTTATTTGTGGATTACATATATACATAAGAAC |
| | TAGAAAAATGTTTATATTTTCGACTTGACAATGATAGAATGTA |
| | AATGTTGGATATATAGAATAATTTGTTGGAGCTTCTACCATTTT |
| | TTGCTTACCGTCTTAAGGTTGAAGGCGTAGATGTTTATTATCCG |
| | AATAATTCCTTTTTGTCGATAATCAAGCATCAATATTAGATTAG |
| | TAGCCGACCCTCTTCTCTTACCTCTCTAAACAATTTTTCAATAT |
| | TTCCATTTTGGTTGGTAAAAATATTAGAGAACATTTTCTCTATA |
| | GAAATATCATGTGTTTATTCAGAATTGTTATTGACTCCATATAT |
| | TTTTAATAATCCAAAACCTATAAATTTAATAGTTTACATTCACA |
| | ATTATTGCTTGTTGCTGTTCTTTTACCTTGTGTTTCTAATTTTTA |
| | CCGTTATTAAATATGCATGTGATTAGTTGAAATATTAAATCTCT |
| | AGAAATTGAAGGCTGGCTCGAGGTTAGAGATTAAATGGAAAA |
| | ATGAAAGTCAACGAAGCATGGTAGCTGTCAAATACGCTCCAAC |
| | TCAAAGCATTACTGTCATGATGGAGTTCCTATTGATCCATCGTG |
| | AAAAATTGAACGATATGTTGGCACATGTCGGAATACTTAAAAT |
| | ATCGAATTAATATAATTGAATTTAACTTTGAAAATTAATCAAA |
| | TTAACTTTCGAAAAGCGCTACCTGACAATTAAAAATACAAGGA |
| | GTGAGCAGTGAACTTAAACTAATCTATTTCAGATCTCTAGGCTT |
| | TGATAAACTAATTGACAGTTCAACATTTGCAGATCTAACTTCA |
| | ACGTTTGCTTCTTGTAGATCTGCAACAAGGCAAACTACCATTTG |
| | AGAGTAAAGAAAAAGGTATAATATAATGTTTTTCTAGTTTTCA |
| | TTTTACATGTATTTTTGTGATTCACTTGATTTGAATGAAACATA |
| | CATTACAAGAACTAGAAAACGAATTTGTATTTTTGACTTAGAT |
| | AATTATAAAATGTTAGATGATTTTGTCATAATTTGTAGTATTCA |
| | CTCTTGTTTTTAGTAAATATACCTCTTTCTTAAATGTGATTGATT |
| | TACAGATTATATATTTTAGAATTTTTGCTTATTAAAAGAAAAAT |
| | TAATTAGTGGCGGAAATTTGAGGTGAATTTGTGAAGAATGGAA |
| | GAAGTTGGAAATTCGAGAGTTTGTTCATGTGTTTTTTAAGGTAG |
| | AAGAAACAAAAAAAATCCATTTGATCTAAAATTCCAATTGAAA |
| | TGTATTACAAGTTGTTTGGGGTAAAAAATAATGACATGAATGT |
| | GTTTTTCTCAAATGATAATGACATAGATGAGCCAAAGTTTTAA |
| | CTGATGACATAAATGAACCTTTCTTTTTCTACAAAGTTCGATAA |
| | CATATTTGAGCCAAAATTTAGAATTTATAACAGCTGACATTCTT |
| | TTAACTCTTTCTTCTTGCTGTTACTTTTACTTGTGTTTCTAATTC |
| | TTTTATCCTTATTAAATGTGGATGTGATTAGTTTGAAAAATCAA |
| | ATTTTGTAGATATTGAAGGCTGGTTGGATGGAAAAAGGAGAAA |
| | AATCATTGGTATGTCTATTGTCTACCAATACTGTAGTTTCTAGC |
| | GTTAAGAGCCAATATTTTCAACTGAATTTTGCTGATGCATATGT |
| | TTTTGATTTAGCAGCTACTATTTGAGGAACAAAAAACAAGAAT |
| | CACGAATCTTATTGATGATTTCTTGATTCGCTTGAAGCAAATAA |
| | AGAATGAATTCATTGCTTCAAAATTGGATGCCTTTGAAAATCT |
| | AAGAATGGAACTGAGATTCCTAAGAACATTTGTCCTGTTTGGG |
| | AATTCGATGAATTTGGATGACTTTTATGAGAGGATGTCACTGA |
| | GTATAAGCAAATTCGATCAATCTACGGAGTACGTAGATAAAAT |
| | AATCCTAGAGAAATACAACATGGAATGTCTTGCCCCTCTGCTG |
| | CTTGAAGAGATAAGAAATTATTTGAGTTTGAAGAATGATTATG |
| | TAGCCACAACTACAGAGATAAAATTGTTTGAATACCTCATCAG |
| | AAACCTCCATGATCTACCAAAGTATTGTTCTGATTTGCTTCTAC |
| | CACTCATGAGTGAATACAAGATTCTTCGGCAAGTATGCACACA |
| | TCTCAGAGATTTCTATCAGTTGGAATGCAACAAAACAACAAAA |
| | ACAGAATTTCTCTATACTCGGTATCAAGTGACAGCCGATAGAG |
| | TAACACAATTCTGTTTTGATCTTTGGACAGGAATGTATAAATAC |
| | TCTGACAATGAGTATGCCTTCTCTGAATGTTCTTCCAAGATCAC |
| | TTCTCTACTCATTGACATAATCCCTCTTGAGTTGGAGGTTCTAT |
| | ACATTTCTACTTCTAAGCTCATCAAAGAGTCAACGTCAAAAGA |
| | ACTAGAAGGATTTGTTAAGCAAATCTTAAAAGCATCTCCAAGG |
| | ATTCTTCAACATTATCTCATTCATCTCCAAGGACGCATGGCCGG |
| | TGTAGAAGCCGTCAATTTCGCTCCAACTCGAAGCATTAGTGTC |
| | ATGATGGAGTTCCTATTGATCTTTCTCACCGATATGCCAAAGCG |
| | GTTTATCCATCGTGAAAAATTGAACGATATGTTGGCACATGTC |
| | AGAATACTTACAAGGAAAATATCTACTCTGGTGAGTAAACTGT |
| | TGGAGGAGATCTCTGAGGATAATATCAATGAAGCGGACTTTTC |
| | AGCTCCGGACTTTTTGCAAGAAATTGAACAAATGAAGGGAGAT |
| | ATCAGACACATCTTTTTAAAAGCTCCTGAGTCATCTCAACTTCG |
| | GTTTCCTATGGATGATGGTTTCCTCTTCATGAATCTTCTACTCA |
| | GACATTTAAATGATTTACTCATTTCCAATGCTTATTCAGTTTTT |
| | CTCATAAAAAAAGAAATTGGGATGGTGAAAGAAAGCCTTGAA |
| | TTCCTAATATCATCTTTCAGGAAAGTCAGGCAAACATTGGATG |
| | AGAGTACTAGTGGAGTAGTTAAAAATTGTTGGGTGCGTGCTTT |
| | GGATGTGGCATATGAGGCAGAACATATCATTAATTCCATTCTT |
| | GTCAGAGATAAAGCTCTCTCACATCTCCTCTTCTCACTTCCGAG |
| | TGTCACTGATAAGATCAAACTTATCGTGGAACAAGTCACCAGG |
| | TTTCAGCTGGAGGATAAGAATGGGGATGGCCCCCTTGATGCAA |
| | AGTCTTCCTTCGAGCCAACTCAGTCAACTTCATCACCTTTTGTT |
| | GAGGTAACAGTAGGTCACGAGAAAGAAGAATCCCAGATCATT |
| | GACCAGCTCCTTGATGAACATGAATCTGAGCTTGATGTCATTTC |
| | CATTGTCGGAATGCCAGGACTCGGTAAAACTACTCTGGCCAAC |
| | AAAGTGTATAAAGATACATTAGTTGCTAGGCATTTCCATGTCC |
| | GTGCTTGGTGCACTGTTTCGCAAAAGTATAACAAGTCAAAGGT |
| | GTTGCGGGAGATTCTTCAGCAAGTTACTGGCTCGGGAGGAAAA |
| | GAAAGTGAAGATGACCTGGCTGAAAAGCTACGAAGAGCACTA |
| | CTCGATAAAAGGTACCTAATCGTCTTGGATGATGTGTGGGATA |
| | TTGCAACAGGGGAGATGTTAATAGCATGTTTTCCTAAGGGTAA |
| | GAGAGGAAATAGAATCATCTTAACTAGCCGAAGTAGAAAGGT |
| | AGGTTTGAAAGTTAAATGCCGTAGTGATCCTCTCGACCTTAAA |
| | CTTTTAACATCTGAAGAAAGTTGGGATTTATTCGAAAAAAGGG |
| | TATTTGGAGATGAAGGAAGCTGCCCTGCTGAACTGTCCGAAGT |
| | TGGACACCAAATAGTTGAGAAATGTAAGGGTCTTCCCTTGGCT |
| | ATTGTTTTAATTGCTGGAGTAATTGTTAGAGGGAAGAAAAAGG |
| | AAAAGGATTTTTGGCTTAAGATACTGCATAATCTGGATTCCTTT |
| | ATTTCTACCAACATCAATTTGGTTATGCAATTAAGTTATGACCA |
| | TTTACCATGCCACCTGAAGCCGTTGCTGCTTTACTTTGCAACAA |
| | CTCAAAAGAGCCAACAAACTCCAGTCTCTACATTGATGCAGTT |
| | GTGGATGGCCGAAGGGTTTGTGGATCATGATAGTTTAGAGGAA |
| | GTAACTCAAAGTTACTTGGATGCTCTAATTTCCAGTAGCCTGAT |
| | AATGGTGGATCATATCCCCTCCAAGAGTTATTGGTGGACGTCT |
| | TTAATGATCAAGGTTTGCTATGTGCATGATGTTGTGCACGATTT |
| | TTGTTCAGAAAAAGCCAAAAAGGAGAAGTTTCTCAAGTTAATC |
| | AATTCAGGTGATCCATTTCATGCTTCAGATTTCCTACACCATCG |
| | TCTAACCATTCATACTGACAACGGCCAACTCCACAAAAAATGT |
| | GTTTTATTCAATTCTAATAAGTGCTTAGCTGTTAGTAAGCATGT |
| | CATATCTTTGAAAGTGAGTGGTCCACTAGATGAGTTCAGGTAT |
| | ATCTGTCACACAAGACACTTTGGACTTGTTAGAGTGTTGCAAC |
| | TGGATGACATCATTCTGGAAGATTCTTTAATGGAAGAAATAGG |
| | GTCCCTATTTCATTTGAGGTTCTTAAGCATTGAGACTGCTGTAG |
| | GTGTAATAGCTATCCCAGTGTCGTGGTTGAACCTCCAGAATCT |
| | GGAAACGCTGTTGATTTATACAACTTATTCCACCATGGTATTAC |
| | TGCCCAGAATATTGCAACTGTCAAAGCTGAAACATGTGAAAAT |
| | TAAGGAATGTTCTTTCTTTGAAGAGAAAGAGGATATCCAACGT |
| | AGAATATTGGAAGCTGGGAATTCTTCAAACTTGACAACTCTAT |
| | CCGGAGTTGTTATCTCATATTCTGAAGGCATGAGTGATGATGC |
| | TCTGGAGAAGTTCCCAATTCTTCAGCACCTTGATTGCATCATCA |
| | TGGAATCGCAGAATGCTCCTACACACGACTATTGGTTTCTCAA |
| | GCTTGATGTACTTAATAAACTCGAATCATTCGTAGCAAGATAC |
| | AAGCGCAATGGACATCCCTCGTTAAACCGACAACCGTATGGAT |
| | TTCACTTCCCTACAAGCTTGAAAGAGTTACGGTTGACTGGTTTT |
| | TTCCTGAGACCTGATTTGTTGTCAGTAATCGCAGCGTTGCCTGA |
| | GCTTGAGATTATGGAGTTTTCCGGCTGTTATTTCGTGGATACTA |
| | AATGGGACGCAAGTGAGGACATCTATCTAAGTCTTAAGACTTT |
| | GATTTTGCGAGATGTCCATTTATCAGAATGGCAAGTTGAGGGG |
| | GGAACTTTTCCCAAGCTTGAGAAATTAATACTAAAATTTTGTTC |
| | CACACTTGGGGAGATCCCTTGTGCATTTATGGATGTAGAAACT |
| | TTAAAGTCCATTGATTTAAGTTTTGTTGGGCGTAAGCTTCGAGA |
| | TTCAGCCATTGAGATTAAGAAAAATGTAGCAGATTTCACAGGA |
| | GAGGACAGAGTTGATGTCCACACATCACATTTGTTTGCAACCA |
| | ACGTGAAAGAACAGATTATGAGAATGACGGGTATGGGAAATA |
| | TACAGGGAAGGAGAGGAATTTAAAAAGTTTAGAACTTGAAAG |
| | GTTGGGAACTTGAAAAGTACTCTAATGCTTTATTGAAAAAGAT |
| | AATAGTCCCTCATCGGTAATGGAAATGAAAATAGGAGAGTTTA |
| | AATAAATAAACACTCCTATTAAATTATTTTTTTTTACAAATAAA |
| | TTTCTGTTCATCTTATTTACTGGTTCTAAAAATCTCAGTTACTTC |
| | GTGTTTCTGAATTATTTATTATAACCAGTAACTTCTGATTTTCTT |
| | AACACAGATTGGCAACAGAGATAACTAAGTTCGTTTAATTTAT |
| | ATAATTTGCATTTGTGTAGTGTGTTCATTGAAGTTGTTAGTAGA |
| | GTTCATTCTTCTTTCTGCACACACTACCACAAAAACAACTTTTT |
| | AGAGGCAATAAACAAATCATCCACTGATTTGACTAGTCGTAAC |
| | ATTATGAGAAAATTCAATGGATTTGTTTTGTATTGCAGGTGGA |
| | GAATGTACTTGTGAAGAACTATAATACTTAGATCCTGAATGTT |
| | TGATTTGTGCTTATACATAGTTGGTGGAGTGATTGCACTGATTC |
| | TCGTGGTTATGAATCGCAGTGGTTGATGAAGTTGGATTTCTCAT |
| | GGAAAAATCACAACAATGAAGAACGAAACAACGCAACGCCTC |
| | CATCTGATTACTGCTTCCAATAAACAAACTTGTTACTGGTTTTC |
| | TCATTGCTGGTTCTGGACGTATTTTGATCTAATGTGTCGTGTGT |
| | TTTCTTTCCTGTTTTTTTGTTAGTTATGAATCTTATTTCGTCTGT |
| | AATCTCTCATATAAGATTCTAGTTTGTTTGTTTTTTTAATTGGA |
| | AGGTTTCAATGCCAAATTGTGAATCAATATAGGTCCCATTTGG |
| | CATGAAAAAGGCAACTAACTATCACTATTGATATGGACAGTTA |
| | ATTAGATAACTACTGTGCCAACAAGCATTGTGGTGGAGAGGCA |
| | GTACTCTCTGATCCTTAATCAGAGGTCTCGCGTTCGAGCCCTAC |
| | CAGATATAGAATCGCCTATGTATGTCTTACTCTCAATGTGATAC |
| | TATTTTTCAATATTAAATATGTATCAAATATAGAATATAATTAT |
| | CGAAACTATCTTAAATCCTTGCAATTAGGTCCTAAACTATTATT |
| | ATTATTATTATATAATAAACTAATAAAGTAACATCAAACTTGC |
| | CATTTTTTATTCTTGTCACCTAATTACATGATTGAATAATGTTTT |
| | TTCCCGACTATTATTGATCCACTAGCAAATATTTATATGGACAG |
| | TAGGAAATTATATAGAAAGACTTTGCTTTAATAATTCAAAATC |
| | ATAGTAAACTTTGCTTAAACTAATCTATTTCACATCTAGTCTTT |
| | AATTGCTTCTTGCTTTTGCTCACTCAATTTTGTCAGTGAAAGTT |
| | TTGTAAATCTGCAAACTACCATTTAGGATGAAGTAACTCTTTTA |
| | ACAGCTGACATTCTTTTAACTCTTTCTTCTTGCTGTTACTTTTAC |
| | TTGTGTTTCTAATTCTTTTATCCTTATTACAAGTGCATGTGATTA |
| | GTTTGAAAAATCAAATTTTGTAGATATTGAAGGCTGGTTGGAT |
| | GGAAAAGGGAGAAAAATCATTGGTATGTCTATTGTCTACCAAT |
| | ACTGTAGTTTCTAGCGTTAAGAGCCAATATTTTCAACTGAATTT |
| | TGCTGATGAATATGTTTTTGATTTAGCAGCTACTATTTGAGGAA |
| | CAAAAAACAAGAATCACGAATCTTATTGATGATTTCTTGATTC |
| | GCTTGAAGCAAATAAAGAATGAATTCATTGCTTCAAAATTGGA |
| | TGCCTTTGAAAATCTAAGAATGGAACTGAGATTCCTAAGAACA |
| | TTTGTCCTGTTTGGGAATTCGATGAATTTGGATGACTTTTATGA |
| | GAGGATGTCACTGAGTATAAGCAAATTCGATCAATCTACGGAG |
| | TACGTAGATAAAATAATCCTAGAGAAATACAACATGGAATGCC |
| | TTGCCCCTCTGCTGCTTGAAGAGATAAGAAATTATTTGAGTTTG |
| | AAGAATGATTATGTAGCCACAACTACAGAGATAAAATTGTTTG |
| | AATACCTCATCAGAAACCTCCATGATCTACCAAAGTATTGTTCT |
| | GATTTGCTTCTACCACTCATGAGTGAATACAAGATTCTTCGGCA |
| | AGTATGCACACATCTCAGAGATTTCTATCAGTTGGAATGCAAC |
| | AAAACAACAAAAACAGAATTTCTCTATACTCGGTATCAAGTGA |
| | CAGTTAATAGAGTAACACAATTCTGTTTTGATCTTTGGACAAG |
| | AAAGTATAAAGACTTTGACAATGAGTATGCCTTCTCTGAATGT |
| | TCTTCCAAGATCACTTCTCTACTCATCGACATAATCCCTCTTGA |
| | GTTGGAGGTTCTACACATTTCTACACATTGTTAAGCAAATCCTA |
| | AAAGCATCTCCAAGGATTCTTCAAAAGCATCTCATTCATCTCC |
| | AAGGACGCATGGTAGCCGTCAGTTACTCTACAACTCAAAGCAT |
| | TAATGTCATGATGGAGTTCCTATTGATCTTTCTCACTGATATAC |
| | CAAAGCGCTTTATCCATCGTGGCAAATTGAACTCTATGTTGGC |
| | ACATGTCGGACTACTTACAAGGAAGATATCTATTCTCATGGAA |
| | GAGAGCTCTAATATGAATGAAGCAGACTTTTCAGCTACATACT |
| | TGTTGCAAGAAATCGAACGAATGAAGAGAGATATCAAACAGA |
| | TTATTTTGAAAGCGCCAGAGTCATCCCAACTTTGCCTTCCTATG |
| | GATGATGGTTTCCTCTTCATGAATCTTCTACTCCGACATTTAAA |
| | TGATTTACTCACTTCCAATTCTTATTCAGTTTCTCTGATAAAAA |
| | AAGAAATTGAGATGGTGAAACAATGCCTTGAATTCCTAACAAC |
| | ATCTTTCAGGCAAACATTGGATGAGAGCACTAGTGGAGTTGTT |
| | AAAGATTGTTGGATGCGTGCTTTGGATG |
| SEQ ID NO: | ATGGAAAAAGGAGAAAAATCATTGCTACTATTTGAGGAACAA |
| 2: | AAAACAAGAATCACGAATCTTATTGATGATTTCTTGATTCGCTT |
| CDS MeR1 | GAAGCAAATAAAGAATGAATTCATTGCTTCAAAATTGGATGCC |
| (isoform 1) | TTTGAAAATCTAAGAATGGAACTGAGATTCCTAAGAACATTTG |
| | TCCTGTTTGGGAATTCGATGAATTTGGATGACTTTTATGAGAGG |
| | ATGTCACTGAGTATAAGCAAATTCGATCAATCTACGGAGTACG |
| | TAGATAAAATAATCCTAGAGAAATACAACATGGAATGTCTTGC |
| | CCCTCTGCTGCTTGAAGAGATAAGAAATTATTTGAGTTTGAAG |
| | AATGATTATGTAGCCACAACTACAGAGATAAAATTGTTTGAAT |
| | ACCTCATCAGAAACCTCCATGATCTACCAAAGTATTGTTCTGAT |
| | TTGCTTCTACCACTCATGAGTGAATACAAGATTCTTCGGCAAGT |
| | ATGCACACATCTCAGAGATTTCTATCAGTTGGAATGCAACAAA |
| | ACAACAAAAACAGAATTTCTCTATACTCGGTATCAAGTGACAG |
| | CCGATAGAGTAACACAATTCTGTTTTGATCTTTGGACAGGAAT |
| | GTATAAATACTCTGACAATGAGTATGCCTTCTCTGAATGTTCTT |
| | CCAAGATCACTTCTCTACTCATTGACATAATCCCTCTTGAGTTG |
| | GAGGTTCTATACATTTCTACTTCTAAGCTCATCAAAGAGTCAAC |
| | GTCAAAAGAACTAGAAGGATTTGTTAAGCAAATCTTAAAAGCA |
| | TCTCCAAGGATTCTTCAACATTATCTCATTCATCTCCAAGGACG |
| | CATGGCCGGTGTAGAAGCCGTCAATTTCGCTCCAACTCGAAGC |
| | ATTAGTGTCATGATGGAGTTCCTATTGATCTTTCTCACCGATAT |
| | GCCAAAGCGGTTTATCCATCGTGAAAAATTGAACGATATGTTG |
| | GCACATGTCAGAATACTTACAAGGAAAATATCTACTCTGGTGA |
| | GTAAACTGTTGGAGGAGATCTCTGAGGATAATATCAATGAAGC |
| | GGACTTTTCAGCTCCGGACTTTTTGCAAGAAATTGAACAAATG |
| | AAGGGAGATATCAGACACATCTTTTTAAAAGCTCCTGAGTCAT |
| | CTCAACTTCGGTTTCCTATGGATGATGGTTTCCTCTTCATGAAT |
| | CTTCTACTCAGACATTTAAATGATTTACTCATTTCCAATGCTTA |
| | TTCAGTTTTTCTCATAAAAAAAGAAATTGGGATGGTGAAAGAA |
| | AGCCTTGAATTCCTAATATCATCTTTCAGGAAAGTCAGGCAAA |
| | CATTGGATGAGAGTACTAGTGGAGTAGTTAAAAATTGTTGGGT |
| | GCGTGCTTTGGATGTGGCATATGAGGCAGAACATATCATTAAT |
| | TCCATTCTTGTCAGAGATAAAGCTCTCTCACATCTCCTCTTCTC |
| | ACTTCCGAGTGTCACTGATAAGATCAAACTTATCGTGGAACAA |
| | GTCACCAGGTTTCAGCTGGAGGATAAGAATGGGGATGGCCCCC |
| | TTGATGCAAAGTCTTCCTTCGAGCCAACTCAGTCAACTTCATCA |
| | CCTTTTGTTGAGGTAACAGTAGGTCACGAGAAAGAAGAATCCC |
| | AGATCATTGACCAGCTCCTTGATGAACATGAATCTGAGCTTGA |
| | TGTCATTTCCATTGTCGGAATGCCAGGACTCGGTAAAACTACT |
| | CTGGCCAACAAAGTGTATAAAGATACATTAGTTGCTAGGCATT |
| | TCCATGTCCGTGCTTGGTGCACTGTTTCGCAAAAGTATAACAA |
| | GTCAAAGGTGTTGCGGGAGATTCTTCAGCAAGTTACTGGCTCG |
| | GGAGGAAAAGAAAGTGAAGATGACCTGGCTGAAAAGCTACGA |
| | AGAGCACTACTCGATAAAAGGTACCTAATCGTCTTGGATGATG |
| | TGTGGGATATTGCAACAGGGGAGATGTTAATAGCATGTTTTCC |
| | TAAGGGTAAGAGAGGAAATAGAATCATCTTAACTAGCCGAAG |
| | TAGAAAGGTAGGTTTGAAAGTTAAATGCCGTAGTGATCCTCTC |
| | GACCTTAAACTTTTAACATCTGAAGAAAGTTGGGATTTATTCG |
| | AAAAAAGGGTATTTGGAGATGAAGGAAGCTGCCCTGCTGAAC |
| | TGTCCGAAGTTGGACACCAAATAGTTGAGAAATGTAAGGGTCT |
| | TCCCTTGGCTATTGTTTTAATTGCTGGAGTAATTGTTAGAGGGA |
| | AGAAAAAGGAAAAGGATTTTTGGCTTAAGATACTGCATAATCT |
| | GGATTCCTTTATTTCTACCAACATCAATTTGGTTATGCAATTAA |
| | GTTATGACCATTTACCATGCCACCTGAAGCCGTTGCTGCTTTAC |
| | TTTGCAACAACTCAAAAGAGCCAACAAACTCCAGTCTCTACAT |
| | TGATGCAGTTGTGGATGGCCGAAGGGTTTGTGGATCATGATAG |
| | TTTAGAGGAAGTAACTCAAAGTTACTTGGATGCTCTAATTTCC |
| | AGTAGCCTGATAATGGTGGATCATATCCCCTCCAAGAGTTATT |
| | GGTGGACGTCTTTAATGATCAAGGTTTGCTATGTGCATGATGTT |
| | GTGCACGATTTTTGTTCAGAAAAAGCCAAAAAGGAGAAGTTTC |
| | TCAAGTTAATCAATTCAGGTGATCCATTTCATGCTTCAGATTTC |
| | CTACACCATCGTCTAACCATTCATACTGACAACGGCCAACTCC |
| | ACAAAAAATGTGTTTTATTCAATTCTAATAAGTGCTTAGCTGTT |
| | AGTAAGCATGTCATATCTTTGAAAGTGAGTGGTCCACTAGATG |
| | AGTTCAGGTATATCTGTCACACAAGACACTTTGGACTTGTTAG |
| | AGTGTTGCAACTGGATGACATCATTCTGGAAGATTCTTTAATG |
| | GAAGAAATAGGGTCCCTATTTCATTTGAGGTTCTTAAGCATTG |
| | AGACTGCTGTAGGTGTAATAGCTATCCCAGTGTCGTGGTTGAA |
| | CCTCCAGAATCTGGAAACGCTGTTGATTTATACAACTTATTCCA |
| | CCATGGTATTACTGCCCAGAATATTGCAACTGTCAAAGCTGAA |
| | ACATGTGAAAATTAAGGAATGTTCTTTCTTTGAAGAGAAAGAG |
| | GATATCCAACGTAGAATATTGGAAGCTGGGAATTCTTCAAACT |
| | TGACAACTCTATCCGGAGTTGTTATCTCATATTCTGAAGGCATG |
| | AGTGATGATGCTCTGGAGAAGTTCCCAATTCTTCAGCACCTTG |
| | ATTGCATCATCATGGAATCGCAGAATGCTCCTACACACGACTA |
| | TTGGTTTCTCAAGCTTGATGTACTTAATAAACTCGAATCATTCG |
| | TAGCAAGATACAAGCGCAATGGACATCCCTCGTTAAACCGACA |
| | ACCGTATGGATTTCACTTCCCTACAAGCTTGAAAGAGTTACGG |
| | TTGACTGGTTTTTTCCTGAGACCTGATTTGTTGTCAGTAATCGC |
| | AGCGTTGCCTGAGCTTGAGATTATGGAGTTTTCCGGCTGTTATT |
| | TCGTGGATACTAAATGGGACGCAAGTGAGGACATCTATCTAAG |
| | TCTTAAGACTTTGATTTTGCGAGATGTCCATTTATCAGAATGGC |
| | AAGTTGAGGGGGGAACTTTTCCCAAGCTTGAGAAATTAATACT |
| | AAAATTTTGTTCCACACTTGGGGAGATCCCTTGTGCATTTATGG |
| | ATGTAGAAACTTTAAAGTCCATTGATTTAAGTTTTGTTGGGCGT |
| | AAGCTTCGAGATTCAGCCATTGAGATTAAGAAAAATGTAGCAG |
| | ATTTCACAGGAGAGGACAGAGTTGATGTCCACACATCACATTT |
| | GTTTGCAACCAACGTGAAAGAACAGATTATGAGAATGACGGG |
| | TATGGGAAATATACAGGGAAGGAGAGGAATTTAA |
| SEQ ID NO: | ATGGAAAAAGGAGAAAAATCATTGCAGCTACTATTTGAGGAA |
| 3: | CAAAAAACAAGAATCACGAATCTTATTGATGATTTCTTGATTC |
| CDS MeR1 | GCTTGAAGCAAATAAAGAATGAATTCATTGCTTCAAAATTGGA |
| (isoform 2) | TGCCTTTGAAAATCTAAGAATGGAACTGAGATTCCTAAGAACA |
| | TTTGTCCTGTTTGGGAATTCGATGAATTTGGATGACTTTTATGA |
| | GAGGATGTCACTGAGTATAAGCAAATTCGATCAATCTACGGAG |
| | TACGTAGATAAAATAATCCTAGAGAAATACAACATGGAATGTC |
| | TTGCCCCTCTGCTGCTTGAAGAGATAAGAAATTATTTGAGTTTG |
| | AAGAATGATTATGTAGCCACAACTACAGAGATAAAATTGTTTG |
| | AATACCTCATCAGAAACCTCCATGATCTACCAAAGTATTGTTCT |
| | GATTTGCTTCTACCACTCATGAGTGAATACAAGATTCTTCGGCA |
| | AGTATGCACACATCTCAGAGATTTCTATCAGTTGGAATGCAAC |
| | AAAACAACAAAAACAGAATTTCTCTATACTCGGTATCAAGTGA |
| | CAGCCGATAGAGTAACACAATTCTGTTTTGATCTTTGGACAGG |
| | AATGTATAAATACTCTGACAATGAGTATGCCTTCTCTGAATGTT |
| | CTTCCAAGATCACTTCTCTACTCATTGACATAATCCCTCTTGAG |
| | TTGGAGGTTCTATACATTTCTACTTCTAAGCTCATCAAAGAGTC |
| | AACGTCAAAAGAACTAGAAGGATTTGTTAAGCAAATCTTAAAA |
| | GCATCTCCAAGGATTCTTCAACATTATCTCATTCATCTCCAAGG |
| | ACGCATGGCCGGTGTAGAAGCCGTCAATTTCGCTCCAACTCGA |
| | AGCATTAGTGTCATGATGGAGTTCCTATTGATCTTTCTCACCGA |
| | TATGCCAAAGCGGTTTATCCATCGTGAAAAATTGAACGATATG |
| | TTGGCACATGTCAGAATACTTACAAGGAAAATATCTACTCTGG |
| | TGAGTAAACTGTTGGAGGAGATCTCTGAGGATAATATCAATGA |
| | AGCGGACTTTTCAGCTCCGGACTTTTTGCAAGAAATTGAACAA |
| | ATGAAGGGAGATATCAGACACATCTTTTTAAAAGCTCCTGAGT |
| | CATCTCAACTTCGGTTTCCTATGGATGATGGTTTCCTCTTCATG |
| | AATCTTCTACTCAGACATTTAAATGATTTACTCATTTCCAATGC |
| | TTATTCAGTTTTTCTCATAAAAAAAGAAATTGGGATGGTGAAA |
| | GAAAGCCTTGAATTCCTAATATCATCTTTCAGGAAAGTCAGGC |
| | AAACATTGGATGAGAGTACTAGTGGAGTAGTTAAAAATTGTTG |
| | GGTGCGTGCTTTGGATGTGGCATATGAGGCAGAACATATCATT |
| | AATTCCATTCTTGTCAGAGATAAAGCTCTCTCACATCTCCTCTT |
| | CTCACTTCCGAGTGTCACTGATAAGATCAAACTTATCGTGGAA |
| | CAAGTCACCAGGTTTCAGCTGGAGGATAAGAATGGGGATGGC |
| | CCCCTTGATGCAAAGTCTTCCTTCGAGCCAACTCAGTCAACTTC |
| | ATCACCTTTTGTTGAGGTAACAGTAGGTCACGAGAAAGAAGAA |
| | TCCCAGATCATTGACCAGCTCCTTGATGAACATGAATCTGAGC |
| | TTGATGTCATTTCCATTGTCGGAATGCCAGGACTCGGTAAAAC |
| | TACTCTGGCCAACAAAGTGTATAAAGATACATTAGTTGCTAGG |
| | CATTTCCATGTCCGTGCTTGGTGCACTGTTTCGCAAAAGTATAA |
| | CAAGTCAAAGGTGTTGCGGGAGATTCTTCAGCAAGTTACTGGC |
| | TCGGGAGGAAAAGAAAGTGAAGATGACCTGGCTGAAAAGCTA |
| | CGAAGAGCACTACTCGATAAAAGGTACCTAATCGTCTTGGATG |
| | ATGTGTGGGATATTGCAACAGGGGAGATGTTAATAGCATGTTT |
| | TCCTAAGGGTAAGAGAGGAAATAGAATCATCTTAACTAGCCGA |
| | AGTAGAAAGGTAGGTTTGAAAGTTAAATGCCGTAGTGATCCTC |
| | TCGACCTTAAACTTTTAACATCTGAAGAAAGTTGGGATTTATTC |
| | GAAAAAAGGGTATTTGGAGATGAAGGAAGCTGCCCTGCTGAA |
| | CTGTCCGAAGTTGGACACCAAATAGTTGAGAAATGTAAGGGTC |
| | TTCCCTTGGCTATTGTTTTAATTGCTGGAGTAATTGTTAGAGGG |
| | AAGAAAAAGGAAAAGGATTTTTGGCTTAAGATACTGCATAATC |
| | TGGATTCCTTTATTTCTACCAACATCAATTTGGTTATGCAATTA |
| | AGTTATGACCATTTACCATGCCACCTGAAGCCGTTGCTGCTTTA |
| | CTTTGCAACAACTCAAAAGAGCCAACAAACTCCAGTCTCTACA |
| | TTGATGCAGTTGTGGATGGCCGAAGGGTTTGTGGATCATGATA |
| | GTTTAGAGGAAGTAACTCAAAGTTACTTGGATGCTCTAATTTC |
| | CAGTAGCCTGATAATGGTGGATCATATCCCCTCCAAGAGTTAT |
| | TGGTGGACGTCTTTAATGATCAAGGTTTGCTATGTGCATGATGT |
| | TGTGCACGATTTTTGTTCAGAAAAAGCCAAAAAGGAGAAGTTT |
| | CTCAAGTTAATCAATTCAGGTGATCCATTTCATGCTTCAGATTT |
| | CCTACACCATCGTCTAACCATTCATACTGACAACGGCCAACTC |
| | CACAAAAAATGTGTTTTATTCAATTCTAATAAGTGCTTAGCTGT |
| | TAGTAAGCATGTCATATCTTTGAAAGTGAGTGGTCCACTAGAT |
| | GAGTTCAGGTATATCTGTCACACAAGACACTTTGGACTTGTTA |
| | GAGTGTTGCAACTGGATGACATCATTCTGGAAGATTCTTTAAT |
| | GGAAGAAATAGGGTCCCTATTTCATTTGAGGTTCTTAAGCATT |
| | GAGACTGCTGTAGGTGTAATAGCTATCCCAGTGTCGTGGTTGA |
| | ACCTCCAGAATCTGGAAACGCTGTTGATTTATACAACTTATTCC |
| | ACCATGGTATTACTGCCCAGAATATTGCAACTGTCAAAGCTGA |
| | AACATGTGAAAATTAAGGAATGTTCTTTCTTTGAAGAGAAAGA |
| | GGATATCCAACGTAGAATATTGGAAGCTGGGAATTCTTCAAAC |
| | TTGACAACTCTATCCGGAGTTGTTATCTCATATTCTGAAGGCAT |
| | GAGTGATGATGCTCTGGAGAAGTTCCCAATTCTTCAGCACCTT |
| | GATTGCATCATCATGGAATCGCAGAATGCTCCTACACACGACT |
| | ATTGGTTTCTCAAGCTTGATGTACTTAATAAACTCGAATCATTC |
| | GTAGCAAGATACAAGCGCAATGGACATCCCTCGTTAAACCGAC |
| | AACCGTATGGATTTCACTTCCCTACAAGCTTGAAAGAGTTACG |
| | GTTGACTGGTTTTTTCCTGAGACCTGATTTGTTGTCAGTAATCG |
| | CAGCGTTGCCTGAGCTTGAGATTATGGAGTTTTCCGGCTGTTAT |
| | TTCGTGGATACTAAATGGGACGCAAGTGAGGACATCTATCTAA |
| | GTCTTAAGACTTTGATTTTGCGAGATGTCCATTTATCAGAATGG |
| | CAAGTTGAGGGGGGAACTTTTCCCAAGCTTGAGAAATTAATAC |
| | TAAAATTTTGTTCCACACTTGGGGAGATCCCTTGTGCATTTATG |
| | GATGTAGAAACTTTAAAGTCCATTGATTTAAGTTTTGTTGGGCG |
| | TAAGCTTCGAGATTCAGCCATTGAGATTAAGAAAAATGTAGCA |
| | GATTTCACAGGAGAGGACAGAGTTGATGTCCACACATCACATT |
| | TGTTTGCAACCAACGTGAAAGAACAGATTATGAGAATGACGG |
| | GTATGGGAAATATACAGGGAAGGAGAGGAATTTAA |
| SEQ ID NO: | ATGGAAAAAGGAGAAAAATCATTGCTACTATTTGAGGAACAA |
| 4: | AAAACAAGAATCACGAATCTTATTGATGATTTCTTGATTCGCTT |
| CDS MeR1 | GAAGCAAATAAAGAATGAATTCATTGCTTCAAAATTGGATGCC |
| (isoform 3) | TTTGAAAATCTAAGAATGGAACTGAGATTCCTAAGAACATTTG |
| | TCCTGTTTGGGAATTCGATGAATTTGGATGACTTTTATGAGAGG |
| | ATGTCACTGAGTATAAGCAAATTCGATCAATCTACGGAGTACG |
| | TAGATAAAATAATCCTAGAGAAATACAACATGGAATGTCTTGC |
| | CCCTCTGCTGCTTGAAGAGATAAGAAATTATTTGAGTTTGAAG |
| | AATGATTATGTAGCCACAACTACAGAGATAAAATTGTTTGAAT |
| | ACCTCATCAGAAACCTCCATGATCTACCAAAGTATTGTTCTGAT |
| | TTGCTTCTACCACTCATGAGTGAATACAAGATTCTTCGGCAAGT |
| | ATGCACACATCTCAGAGATTTCTATCAGTTGGAATGCAACAAA |
| | ACAACAAAAACAGAATTTCTCTATACTCGGTATCAAGTGACAG |
| | CCGATAGAGTAACACAATTCTGTTTTGATCTTTGGACAGGAAT |
| | GTATAAATACTCTGACAATGAGTATGCCTTCTCTGAATGTTCTT |
| | CCAAGATCACTTCTCTACTCATTGACATAATCCCTCTTGAGTTG |
| | GAGGTTCTATACATTTCTACTTCTAAGCTCATCAAAGAGTCAAC |
| | GTCAAAAGAACTAGAAGGATTTGTTAAGCAAATCTTAAAAGCA |
| | TCTCCAAGGATTCTTCAACATTATCTCATTCATCTCCAAGGACG |
| | CATGGCCGGTGTAGAAGCCGTCAATTTCGCTCCAACTCGAAGC |
| | ATTAGTGTCATGATGGAGTTCCTATTGATCTTTCTCACCGATAT |
| | GCCAAAGCGGTTTATCCATCGTGAAAAATTGAACGATATGTTG |
| | GCACATGTCAGAATACTTACAAGGAAAATATCTACTCTGGTGA |
| | GTAAACTGTTGGAGGAGATCTCTGAGGATAATATCAATGAAGC |
| | GGACTTTTCAGCTCCGGACTTTTTGCAAGAAATTGAACAAATG |
| | AAGGGAGATATCAGACACATCTTTTTAAAAGCTCCTGAGTCAT |
| | CTCAACTTCGGTTTCCTATGGATGATGGTTTCCTCTTCATGAAT |
| | CTTCTACTCAGACATTTAAATGATTTACTCATTTCCAATGCTTA |
| | TTCAGTTTTTCTCATAAAAAAAGAAATTGGGATGGTGAAAGAA |
| | AGCCTTGAATTCCTAATATCATCTTTCAGGAAAGTCAGGCAAA |
| | CATTGGATGAGAGTACTAGTGGAGTAGTTAAAAATTGTTGGGT |
| | GCGTGCTTTGGATGTGGCATATGAGGCAGAACATATCATTAAT |
| | TCCATTCTTGTCAGAGATAAAGCTCTCTCACATCTCCTCTTCTC |
| | ACTTCCGAGTGTCACTGATAAGATCAAACTTATCGTGGAACAA |
| | GTCACCAGGTTTCAGCTGGAGGATAAGAATGGGGATGGCCCCC |
| | TTGATGCAAAGTCTTCCTTCGAGCCAACTCAGTCAACTTCATCA |
| | CCTTTTGTTGAGGTAACAGTAGGTCACGAGAAAGAAGAATCCC |
| | AGATCATTGACCAGCTCCTTGATGAACATGAATCTGAGCTTGA |
| | TGTCATTTCCATTGTCGGAATGCCAGGACTCGGTAAAACTACT |
| | CTGGCCAACAAAGTGTATAAAGATACATTAGTTGCTAGGCATT |
| | TCCATGTCCGTGCTTGGTGCACTGTTTCGCAAAAGTATAACAA |
| | GTCAAAGGTGTTGCGGGAGATTCTTCAGCAAGTTACTGGCTCG |
| | GGAGGAAAAGAAAGTGAAGATGACCTGGCTGAAAAGCTACGA |
| | AGAGCACTACTCGATAAAAGGTACCTAATCGTCTTGGATGATG |
| | TGTGGGATATTGCAACAGGGGAGATGTTAATAGCATGTTTTCC |
| | TAAGGGTAAGAGAGGAAATAGAATCATCTTAACTAGCCGAAG |
| | TAGAAAGGTAGGTTTGAAAGTTAAATGCCGTAGTGATCCTCTC |
| | GACCTTAAACTTTTAACATCTGAAGAAAGTTGGGATTTATTCG |
| | AAAAAAGGGTATTTGGAGATGAAGGAAGCTGCCCTGCTGAAC |
| | TGTCCGAAGTTGGACACCAAATAGTTGAGAAATGTAAGGGTCT |
| | TCCCTTGGCTATTGTTTTAATTGCTGGAGTAATTGTTAGAGGGA |
| | AGAAAAAGGAAAAGGATTTTTGGCTTAAGATACTGCATAATCT |
| | GGATTCCTTTATTTCTACCAACATCAATTTGGTTATGCAATTAA |
| | GTTATGACCATTTACCATGCCACCTGAAGCCGTTGCTGCTTTAC |
| | TTTGCAACAACTCAAAAGAGCCAACAAACTCCAGTCTCTACAT |
| | TGATGCAGTTGTGGATGGCCGAAGGGTTTGTGGATCATGATAG |
| | TTTAGAGGAAGTAACTCAAAGTTACTTGGATGCTCTAATTTCC |
| | AGTAGCCTGATAATGGTGGATCATATCCCCTCCAAGAGTTATT |
| | GGTGGACGTCTTTAATGATCAAGGTTTGCTATGTGCATGATGTT |
| | GTGCACGATTTTTGTTCAGAAAAAGCCAAAAAGGAGAAGTTTC |
| | TCAAGTTAATCAATTCAGGTGATCCATTTCATGCTTCAGATTTC |
| | CTACACCATCGTCTAACCATTCATACTGACAACGGCCAACTCC |
| | ACAAAAAATGTGTTTTATTCAATTCTAATAAGTGCTTAGCTGTT |
| | AGTAAGCATGTCATATCTTTGAAAGTGAGTGGTCCACTAGATG |
| | AGTTCAGGTATATCTGTCACACAAGACACTTTGGACTTGTTAG |
| | AGTGTTGCAACTGGATGACATCATTCTGGAAGATTCTTTAATG |
| | GAAGAAATAGGGTCCCTATTTCATTTGAGGTTCTTAAGCATTG |
| | AGACTGCTGTAGGTGTAATAGCTATCCCAGTGTCGTGGTTGAA |
| | CCTCCAGAATCTGGAAACGCTGTTGATTTATACAACTTATTCCA |
| | CCATGGTATTACTGCCCAGAATATTGCAACTGTCAAAGCTGAA |
| | ACATGTGAAAATTAAGGAATGTTCTTTCTTTGAAGAGAAAGAG |
| | GATATCCAACGTAGAATATTGGAAGCTGGGAATTCTTCAAACT |
| | TGACAACTCTATCCGGAGTTGTTATCTCATATTCTGAAGGCATG |
| | AGTGATGATGCTCTGGAGAAGTTCCCAATTCTTCAGCACCTTG |
| | ATTGCATCATCATGGAATCGCAGAATGCTCCTACACACGACTA |
| | TTGGTTTCTCAAGCTTGATGTACTTAATAAACTCGAATCATTCG |
| | TAGCAAGATACAAGCGCAATGGACATCCCTCGTTAAACCGACA |
| | ACCGTATGGATTTCACTTCCCTACAAGCTTGAAAGAGTTACGG |
| | TTGACTGGTTTTTTCCTGAGACCTGATTTGTTGTCAGTAATCGC |
| | AGCGTTGCCTGAGCTTGAGATTATGGAGTTTTCCGGCTGTTATT |
| | TCGTGGATACTAAATGGGACGCAAGTGAGGACATCTATCTAAG |
| | TCTTAAGACTTTGATTTTGCGAGATGTCCATTTATCAGAATGGC |
| | AAGTTGAGGGGGGAACTTTTCCCAAGCTTGAGAAATTAATACT |
| | AAAATTTTGTTCCACACTTGGGGAGATCCCTTGTGCATTTATGG |
| | ATGTAGAAACTTTAAAGTCCATTGATTTAAGTTTTGTTGGGCGT |
| | AAGCTTCGAGATTCAGCCATTGAGATTAAGAAAAATGTAGCAG |
| | ATTTCACAGGAGAGGACAGAGTTGATGTCCACACATCACATTT |
| | GTTTGCAACCAACGTGAAAGAACAGATTATGAGAATGACGGG |
| | TGGAGAATGTACTTGTGAAGAACTATAA |
| SEQ ID NO: | ATGGAAAAAGGAGAAAAATCATTGCAGCTACTATTTGAGGAA |
| 5 | CAAAAAACAAGAATCACGAATCTTATTGATGATTTCTTGATTC |
| CDS MeR1 | GCTTGAAGCAAATAAAGAATGAATTCATTGCTTCAAAATTGGA |
| (isoform 4) | TGCCTTTGAAAATCTAAGAATGGAACTGAGATTCCTAAGAACA |
| | TTTGTCCTGTTTGGGAATTCGATGAATTTGGATGACTTTTATGA |
| | GAGGATGTCACTGAGTATAAGCAAATTCGATCAATCTACGGAG |
| | TACGTAGATAAAATAATCCTAGAGAAATACAACATGGAATGTC |
| | TTGCCCCTCTGCTGCTTGAAGAGATAAGAAATTATTTGAGTTTG |
| | AAGAATGATTATGTAGCCACAACTACAGAGATAAAATTGTTTG |
| | AATACCTCATCAGAAACCTCCATGATCTACCAAAGTATTGTTCT |
| | GATTTGCTTCTACCACTCATGAGTGAATACAAGATTCTTCGGCA |
| | AGTATGCACACATCTCAGAGATTTCTATCAGTTGGAATGCAAC |
| | AAAACAACAAAAACAGAATTTCTCTATACTCGGTATCAAGTGA |
| | CAGCCGATAGAGTAACACAATTCTGTTTTGATCTTTGGACAGG |
| | AATGTATAAATACTCTGACAATGAGTATGCCTTCTCTGAATGTT |
| | CTTCCAAGATCACTTCTCTACTCATTGACATAATCCCTCTTGAG |
| | TTGGAGGTTCTATACATTTCTACTTCTAAGCTCATCAAAGAGTC |
| | AACGTCAAAAGAACTAGAAGGATTTGTTAAGCAAATCTTAAAA |
| | GCATCTCCAAGGATTCTTCAACATTATCTCATTCATCTCCAAGG |
| | ACGCATGGCCGGTGTAGAAGCCGTCAATTTCGCTCCAACTCGA |
| | AGCATTAGTGTCATGATGGAGTTCCTATTGATCTTTCTCACCGA |
| | TATGCCAAAGCGGTTTATCCATCGTGAAAAATTGAACGATATG |
| | TTGGCACATGTCAGAATACTTACAAGGAAAATATCTACTCTGG |
| | TGAGTAAACTGTTGGAGGAGATCTCTGAGGATAATATCAATGA |
| | AGCGGACTTTTCAGCTCCGGACTTTTTGCAAGAAATTGAACAA |
| | ATGAAGGGAGATATCAGACACATCTTTTTAAAAGCTCCTGAGT |
| | CATCTCAACTTCGGTTTCCTATGGATGATGGTTTCCTCTTCATG |
| | AATCTTCTACTCAGACATTTAAATGATTTACTCATTTCCAATGC |
| | TTATTCAGTTTTTCTCATAAAAAAAGAAATTGGGATGGTGAAA |
| | GAAAGCCTTGAATTCCTAATATCATCTTTCAGGAAAGTCAGGC |
| | AAACATTGGATGAGAGTACTAGTGGAGTAGTTAAAAATTGTTG |
| | GGTGCGTGCTTTGGATGTGGCATATGAGGCAGAACATATCATT |
| | AATTCCATTCTTGTCAGAGATAAAGCTCTCTCACATCTCCTCTT |
| | CTCACTTCCGAGTGTCACTGATAAGATCAAACTTATCGTGGAA |
| | CAAGTCACCAGGTTTCAGCTGGAGGATAAGAATGGGGATGGC |
| | CCCCTTGATGCAAAGTCTTCCTTCGAGCCAACTCAGTCAACTTC |
| | ATCACCTTTTGTTGAGGTAACAGTAGGTCACGAGAAAGAAGAA |
| | TCCCAGATCATTGACCAGCTCCTTGATGAACATGAATCTGAGC |
| | TTGATGTCATTTCCATTGTCGGAATGCCAGGACTCGGTAAAAC |
| | TACTCTGGCCAACAAAGTGTATAAAGATACATTAGTTGCTAGG |
| | CATTTCCATGTCCGTGCTTGGTGCACTGTTTCGCAAAAGTATAA |
| | CAAGTCAAAGGTGTTGCGGGAGATTCTTCAGCAAGTTACTGGC |
| | TCGGGAGGAAAAGAAAGTGAAGATGACCTGGCTGAAAAGCTA |
| | CGAAGAGCACTACTCGATAAAAGGTACCTAATCGTCTTGGATG |
| | ATGTGTGGGATATTGCAACAGGGGAGATGTTAATAGCATGTTT |
| | TCCTAAGGGTAAGAGAGGAAATAGAATCATCTTAACTAGCCGA |
| | AGTAGAAAGGTAGGTTTGAAAGTTAAATGCCGTAGTGATCCTC |
| | TCGACCTTAAACTTTTAACATCTGAAGAAAGTTGGGATTTATTC |
| | GAAAAAAGGGTATTTGGAGATGAAGGAAGCTGCCCTGCTGAA |
| | CTGTCCGAAGTTGGACACCAAATAGTTGAGAAATGTAAGGGTC |
| | TTCCCTTGGCTATTGTTTTAATTGCTGGAGTAATTGTTAGAGGG |
| | AAGAAAAAGGAAAAGGATTTTTGGCTTAAGATACTGCATAATC |
| | TGGATTCCTTTATTTCTACCAACATCAATTTGGTTATGCAATTA |
| | AGTTATGACCATTTACCATGCCACCTGAAGCCGTTGCTGCTTTA |
| | CTTTGCAACAACTCAAAAGAGCCAACAAACTCCAGTCTCTACA |
| | TTGATGCAGTTGTGGATGGCCGAAGGGTTTGTGGATCATGATA |
| | GTTTAGAGGAAGTAACTCAAAGTTACTTGGATGCTCTAATTTC |
| | CAGTAGCCTGATAATGGTGGATCATATCCCCTCCAAGAGTTAT |
| | TGGTGGACGTCTTTAATGATCAAGGTTTGCTATGTGCATGATGT |
| | TGTGCACGATTTTTGTTCAGAAAAAGCCAAAAAGGAGAAGTTT |
| | CTCAAGTTAATCAATTCAGGTGATCCATTTCATGCTTCAGATTT |
| | CCTACACCATCGTCTAACCATTCATACTGACAACGGCCAACTC |
| | CACAAAAAATGTGTTTTATTCAATTCTAATAAGTGCTTAGCTGT |
| | TAGTAAGCATGTCATATCTTTGAAAGTGAGTGGTCCACTAGAT |
| | GAGTTCAGGTATATCTGTCACACAAGACACTTTGGACTTGTTA |
| | GAGTGTTGCAACTGGATGACATCATTCTGGAAGATTCTTTAAT |
| | GGAAGAAATAGGGTCCCTATTTCATTTGAGGTTCTTAAGCATT |
| | GAGACTGCTGTAGGTGTAATAGCTATCCCAGTGTCGTGGTTGA |
| | ACCTCCAGAATCTGGAAACGCTGTTGATTTATACAACTTATTCC |
| | ACCATGGTATTACTGCCCAGAATATTGCAACTGTCAAAGCTGA |
| | AACATGTGAAAATTAAGGAATGTTCTTTCTTTGAAGAGAAAGA |
| | GGATATCCAACGTAGAATATTGGAAGCTGGGAATTCTTCAAAC |
| | TTGACAACTCTATCCGGAGTTGTTATCTCATATTCTGAAGGCAT |
| | GAGTGATGATGCTCTGGAGAAGTTCCCAATTCTTCAGCACCTT |
| | GATTGCATCATCATGGAATCGCAGAATGCTCCTACACACGACT |
| | ATTGGTTTCTCAAGCTTGATGTACTTAATAAACTCGAATCATTC |
| | GTAGCAAGATACAAGCGCAATGGACATCCCTCGTTAAACCGAC |
| | AACCGTATGGATTTCACTTCCCTACAAGCTTGAAAGAGTTACG |
| | GTTGACTGGTTTTTTCCTGAGACCTGATTTGTTGTCAGTAATCG |
| | CAGCGTTGCCTGAGCTTGAGATTATGGAGTTTTCCGGCTGTTAT |
| | TTCGTGGATACTAAATGGGACGCAAGTGAGGACATCTATCTAA |
| | GTCTTAAGACTTTGATTTTGCGAGATGTCCATTTATCAGAATGG |
| | CAAGTTGAGGGGGGAACTTTTCCCAAGCTTGAGAAATTAATAC |
| | TAAAATTTTGTTCCACACTTGGGGAGATCCCTTGTGCATTTATG |
| | GATGTAGAAACTTTAAAGTCCATTGATTTAAGTTTTGTTGGGCG |
| | TAAGCTTCGAGATTCAGCCATTGAGATTAAGAAAAATGTAGCA |
| | GATTTCACAGGAGAGGACAGAGTTGATGTCCACACATCACATT |
| | TGTTTGCAACCAACGTGAAAGAACAGATTATGAGAATGACGG |
| | GTGGAGAATGTACTTGTGAAGAACTATAA |
| SEQ ID NO: | ATGGAAAAAGGAGAAAAATCATTGCTACTATTTGAGGAACAA |
| 6: | AAAACAAGAATCACGAATCTTATTGATGATTTCTTGATTCGCTT |
| CDS MeR1 | GAAGCAAATAAAGAATGAATTCATTGCTTCAAAATTGGATGCC |
| (isoform 5) | TTTGAAAATCTAAGAATGGAACTGAGATTCCTAAGAACATTTG |
| | TCCTGTTTGGGAATTCGATGAATTTGGATGACTTTTATGAGAGG |
| | ATGTCACTGAGTATAAGCAAATTCGATCAATCTACGGAGTACG |
| | TAGATAAAATAATCCTAGAGAAATACAACATGGAATGTCTTGC |
| | CCCTCTGCTGCTTGAAGAGATAAGAAATTATTTGAGTTTGAAG |
| | AATGATTATGTAGCCACAACTACAGAGATAAAATTGTTTGAAT |
| | ACCTCATCAGAAACCTCCATGATCTACCAAAGTATTGTTCTGAT |
| | TTGCTTCTACCACTCATGAGTGAATACAAGATTCTTCGGCAAGT |
| | ATGCACACATCTCAGAGATTTCTATCAGTTGGAATGCAACAAA |
| | ACAACAAAAACAGAATTTCTCTATACTCGGTATCAAGTGACAG |
| | CCGATAGAGTAACACAATTCTGTTTTGATCTTTGGACAGGAAT |
| | GTATAAATACTCTGACAATGAGTATGCCTTCTCTGAATGTTCTT |
| | CCAAGATCACTTCTCTACTCATTGACATAATCCCTCTTGAGTTG |
| | GAGGTTCTATACATTTCTACTTCTAAGCTCATCAAAGAGTCAAC |
| | GTCAAAAGAACTAGAAGGATTTGTTAAGCAAATCTTAAAAGCA |
| | TCTCCAAGGATTCTTCAACATTATCTCATTCATCTCCAAGGACG |
| | CATGGCCGGTGTAGAAGCCGTCAATTTCGCTCCAACTCGAAGC |
| | ATTAGTGTCATGATGGAGTTCCTATTGATCTTTCTCACCGATAT |
| | GCCAAAGCGGTTTATCCATCGTGAAAAATTGAACGATATGTTG |
| | GCACATGTCAGAATACTTACAAGGAAAATATCTACTCTGGTGA |
| | GTAAACTGTTGGAGGAGATCTCTGAGGATAATATCAATGAAGC |
| | GGACTTTTCAGCTCCGGACTTTTTGCAAGAAATTGAACAAATG |
| | AAGGGAGATATCAGACACATCTTTTTAAAAGCTCCTGAGTCAT |
| | CTCAACTTCGGTTTCCTATGGATGATGGTTTCCTCTTCATGAAT |
| | CTTCTACTCAGACATTTAAATGATTTACTCATTTCCAATGCTTA |
| | TTCAGTTTTTCTCATAAAAAAAGAAATTGGGATGGTGAAAGAA |
| | AGCCTTGAATTCCTAATATCATCTTTCAGGAAAGTCAGGCAAA |
| | CATTGGATGAGAGTACTAGTGGAGTAGTTAAAAATTGTTGGGT |
| | GCGTGCTTTGGATGTGGCATATGAGGCAGAACATATCATTAAT |
| | TCCATTCTTGTCAGAGATAAAGCTCTCTCACATCTCCTCTTCTC |
| | ACTTCCGAGTGTCACTGATAAGATCAAACTTATCGTGGAACAA |
| | GTCACCAGGTTTCAGCTGGAGGATAAGAATGGGGATGGCCCCC |
| | TTGATGCAAAGTCTTCCTTCGAGCCAACTCAGTCAACTTCATCA |
| | CCTTTTGTTGAGGTAACAGTAGGTCACGAGAAAGAAGAATCCC |
| | AGATCATTGACCAGCTCCTTGATGAACATGAATCTGAGCTTGA |
| | TGTCATTTCCATTGTCGGAATGCCAGGACTCGGTAAAACTACT |
| | CTGGCCAACAAAGTGTATAAAGATACATTAGTTGCTAGGCATT |
| | TCCATGTCCGTGCTTGGTGCACTGTTTCGCAAAAGTATAACAA |
| | GTCAAAGGTGTTGCGGGAGATTCTTCAGCAAGTTACTGGCTCG |
| | GGAGGAAAAGAAAGTGAAGATGACCTGGCTGAAAAGCTACGA |
| | AGAGCACTACTCGATAAAAGGTGGAGAATGTACTTGTGA |
| SEQ ID NO: | ATGGAAAAAGGAGAAAAATCATTGCAGCTACTATTTGAGGAA |
| 7: | CAAAAAACAAGAATCACGAATCTTATTGATGATTTCTTGATTC |
| CDS MeR1 | GCTTGAAGCAAATAAAGAATGAATTCATTGCTTCAAAATTGGA |
| (isoform 6) | TGCCTTTGAAAATCTAAGAATGGAACTGAGATTCCTAAGAACA |
| | TTTGTCCTGTTTGGGAATTCGATGAATTTGGATGACTTTTATGA |
| | GAGGATGTCACTGAGTATAAGCAAATTCGATCAATCTACGGAG |
| | TACGTAGATAAAATAATCCTAGAGAAATACAACATGGAATGTC |
| | TTGCCCCTCTGCTGCTTGAAGAGATAAGAAATTATTTGAGTTTG |
| | AAGAATGATTATGTAGCCACAACTACAGAGATAAAATTGTTTG |
| | AATACCTCATCAGAAACCTCCATGATCTACCAAAGTATTGTTCT |
| | GATTTGCTTCTACCACTCATGAGTGAATACAAGATTCTTCGGCA |
| | AGTATGCACACATCTCAGAGATTTCTATCAGTTGGAATGCAAC |
| | AAAACAACAAAAACAGAATTTCTCTATACTCGGTATCAAGTGA |
| | CAGCCGATAGAGTAACACAATTCTGTTTTGATCTTTGGACAGG |
| | AATGTATAAATACTCTGACAATGAGTATGCCTTCTCTGAATGTT |
| | CTTCCAAGATCACTTCTCTACTCATTGACATAATCCCTCTTGAG |
| | TTGGAGGTTCTATACATTTCTACTTCTAAGCTCATCAAAGAGTC |
| | AACGTCAAAAGAACTAGAAGGATTTGTTAAGCAAATCTTAAAA |
| | GCATCTCCAAGGATTCTTCAACATTATCTCATTCATCTCCAAGG |
| | ACGCATGGCCGGTGTAGAAGCCGTCAATTTCGCTCCAACTCGA |
| | AGCATTAGTGTCATGATGGAGTTCCTATTGATCTTTCTCACCGA |
| | TATGCCAAAGCGGTTTATCCATCGTGAAAAATTGAACGATATG |
| | TTGGCACATGTCAGAATACTTACAAGGAAAATATCTACTCTGG |
| | TGAGTAAACTGTTGGAGGAGATCTCTGAGGATAATATCAATGA |
| | AGCGGACTTTTCAGCTCCGGACTTTTTGCAAGAAATTGAACAA |
| | ATGAAGGGAGATATCAGACACATCTTTTTAAAAGCTCCTGAGT |
| | CATCTCAACTTCGGTTTCCTATGGATGATGGTTTCCTCTTCATG |
| | AATCTTCTACTCAGACATTTAAATGATTTACTCATTTCCAATGC |
| | TTATTCAGTTTTTCTCATAAAAAAAGAAATTGGGATGGTGAAA |
| | GAAAGCCTTGAATTCCTAATATCATCTTTCAGGAAAGTCAGGC |
| | AAACATTGGATGAGAGTACTAGTGGAGTAGTTAAAAATTGTTG |
| | GGTGCGTGCTTTGGATGTGGCATATGAGGCAGAACATATCATT |
| | AATTCCATTCTTGTCAGAGATAAAGCTCTCTCACATCTCCTCTT |
| | CTCACTTCCGAGTGTCACTGATAAGATCAAACTTATCGTGGAA |
| | CAAGTCACCAGGTTTCAGCTGGAGGATAAGAATGGGGATGGC |
| | CCCCTTGATGCAAAGTCTTCCTTCGAGCCAACTCAGTCAACTTC |
| | ATCACCTTTTGTTGAGGTAACAGTAGGTCACGAGAAAGAAGAA |
| | TCCCAGATCATTGACCAGCTCCTTGATGAACATGAATCTGAGC |
| | TTGATGTCATTTCCATTGTCGGAATGCCAGGACTCGGTAAAAC |
| | TACTCTGGCCAACAAAGTGTATAAAGATACATTAGTTGCTAGG |
| | CATTTCCATGTCCGTGCTTGGTGCACTGTTTCGCAAAAGTATAA |
| | CAAGTCAAAGGTGTTGCGGGAGATTCTTCAGCAAGTTACTGGC |
| | TCGGGAGGAAAAGAAAGTGAAGATGACCTGGCTGAAAAGCTA |
| | CGAAGAGCACTACTCGATAAAAGGTGGAGAATGTACTTGTGA |
| SEQ ID NO: | ATGGAAAAAGGAGAAAAATCATTGCTACTATTTGAGGAACAA |
| 8: | AAAACAAGAATCACGAATCTTATTGATGATTTCTTGATTCGCTT |
| CDS MeR1 | GAAGCAAATAAAGAATGAATTCATTGCTTCAAAATTGGATGCC |
| (isoform 7) | TTTGAAAATCTAAGAATGGAACTGAGATTCCTAAGAACATTTG |
| | TCCTGTTTGGGAATTCGATGAATTTGGATGACTTTTATGAGAGG |
| | ATGTCACTGAGTATAAGCAAATTCGATCAATCTACGGAGTACG |
| | TAGATAAAATAATCCTAGAGAAATACAACATGGAATGTCTTGC |
| | CCCTCTGCTGCTTGAAGAGATAAGAAATTATTTGAGTTTGAAG |
| | AATGATTATGTAGCCACAACTACAGAGATAAAATTGTTTGAAT |
| | ACCTCATCAGAAACCTCCATGATCTACCAAAGTATTGTTCTGAT |
| | TTGCTTCTACCACTCATGAGTGAATACAAGATTCTTCGGCAAGT |
| | ATGCACACATCTCAGAGATTTCTATCAGTTGGAATGCAACAAA |
| | ACAACAAAAACAGAATTTCTCTATACTCGGTATCAAGTGACAG |
| | CCGATAGAGTAACACAATTCTGTTTTGATCTTTGGACAGGAAT |
| | GTATAAATACTCTGACAATGAGTATGCCTTCTCTGAATGTTCTT |
| | CCAAGATCACTTCTCTACTCATTGACATAATCCCTCTTGAGTTG |
| | GAGGTTCTATACATTTCTACTTCTAAGCTCATCAAAGAGTCAAC |
| | GTCAAAAGAACTAGAAGGATTTGTTAAGCAAATCTTAAAAGCA |
| | TCTCCAAGGATTCTTCAACATTATCTCATTCATCTCCAAGGACG |
| | CATGGCCGGTGTAGAAGCCGTCAATTTCGCTCCAACTCGAAGC |
| | ATTAGTGTCATGATGGAGTTCCTATTGATCTTTCTCACCGATAT |
| | GCCAAAGCGGTTTATCCATCGTGAAAAATTGAACGATATGTTG |
| | GCACATGTCAGAATACTTACAAGGAAAATATCTACTCTGGTGA |
| | GTAAACTGTTGGAGGAGATCTCTGAGGATAATATCAATGAAGC |
| | GGACTTTTCAGCTCCGGACTTTTTGCAAGAAATTGAACAAATG |
| | AAGGGAGATATCAGACACATCTTTTTAAAAGCTCCTGAGTCAT |
| | CTCAACTTCGGTTTCCTATGGATGATGGTTTCCTCTTCATGAAT |
| | CTTCTACTCAGACATTTAAATGATTTACTCATTTCCAATGCTTA |
| | TTCAGTTTTTCTCATAAAAAAAGAAATTGGGATGGTGAAAGAA |
| | AGCCTTGAATTCCTAATATCATCTTTCAGGAAAGTCAGGCAAA |
| | CATTGGATGAGAGTACTAGTGGAGTAGTTAAAAATTGTTGGGT |
| | GCGTGCTTTGGATGTGGCATATGAGGCAGAACATATCATTAAT |
| | TCCATTCTTGTCAGAGATAAAGCTCTCTCACATCTCCTCTTCTC |
| | ACTTCCGAGTGTCACTGATAAGATCAAACTTATCGTGGAACAA |
| | GTCACCAGGTTTCAGCTGGAGGATAAGAATGGGGATGGCCCCC |
| | TTGATGCAAAGTCTTCCTTCGAGCCAACTCAGTCAACTTCATCA |
| | CCTTTTGTTGAGGTAACAGTAGGTCACGAGAAAGAAGAATCCC |
| | AGATCATTGACCAGCTCCTTGATGAACATGAATCTGAGCTTGA |
| | TGTCATTTCCATTGTCGGAATGCCAGGACTCGGTAAAACTACT |
| | CTGGCCAACAAAGTGTATAAAGATACATTAGTTGCTAGGCATT |
| | TCCATGTCCGTGCTTGGTGCACTGTTTCGCAAAAGTATAACAA |
| | GTCAAAGGTGTTGCGGGAGATTCTTCAGCAAGTTACTGGCTCG |
| | GGAGGAAAAGAAAGTGAAGATGACCTGGCTGAAAAGCTACGA |
| | AGAGCACTACTCGATAAAAGGTACCTAATCGTCTTGGATGATG |
| | TGTGGGATATTGCAACAGGGGAGATGTTAATAGCATGTTTTCC |
| | TAAGGGTAAGAGAGGAAATAGAATCATCTTAACTAGCCGAAG |
| | TAGAAAGGTAGGTTTGAAAGTTAAATGCCGTAGTGATCCTCTC |
| | GACCTTAAACTTTTAACATCTGAAGAAAGTTGGGATTTATTCG |
| | AAAAAAGGGTATTTGGAGATGAAGGAAGCTGCCCTGCTGAAC |
| | TGTCCGAAGTTGGACACCAAATAGTTGAGAAATGTAAGGGTCT |
| | TCCCTTGGCTATTGTTTTAATTGCTGGAGTAATTGTTAGAGGGA |
| | AGAAAAAGGAAAAGGATTTTTGGCTTAAGATACTGCATAATCT |
| | GGATTCCTTTATTTCTACCAACATCAATTTGGTTATGCAATTAA |
| | GTTATGACCATTTACCATGCCACCTGAAGCCGTTGCTGCTTTAC |
| | TTTGCAACAACTCAAAAGAGCCAACAAACTCCAGTCTCTACAT |
| | TGATGCAGTTGTGGATGGCCGAAGGGTTTGTGGATCATGATAG |
| | TTTAGAGGAAGTAACTCAAAGTTACTTGGATGCTCTAATTTCC |
| | AGTAGCCTGATAATGGTGGATCATATCCCCTCCAAGAGTTATT |
| | GGTGGACGTCTTTAATGATCAAGGTTTGCTATGTGCATGATGTT |
| | GTGCACGATTTTTGTTCAGAAAAAGCCAAAAAGGAGAAGTTTC |
| | TCAAGTTAATCAATTCAGGTGATCCATTTCATGCTTCAGATTTC |
| | CTACACCATCGTCTAACCATTCATACTGACAACGGCCAACTCC |
| | ACAAAAAATGTGTTTTATTCAATTCTAATAAGTGCTTAGCTGTT |
| | AGTAAGCATGTCATATCTTTGAAAGTGAGTGGTCCACTAGATG |
| | AGTTCAGGTATATCTGTCACACAAGACACTTTGGACTTGTTAG |
| | AGTGTTGCAACTGGATGACATCATTCTGGAAGATTCTTTAATG |
| | GAAGAAATAGGGTCCCTATTTCATTTGAGGTTCTTAAGCATTG |
| | AGACTGCTGTAGGTGTAATAGCTATCCCAGTGTCGTGGTTGAA |
| | CCTCCAGAATCTGGAAACGCTGTTGATTTATACAACTTATTCCA |
| | CCATGGTATTACTGCCCAGAATATTGCAACTGTCAAAGCTGAA |
| | ACATGTGAAAATTAAGGAATGTTCTTTCTTTGAAGAGAAAGAG |
| | GATATCCAACGTAGAATATTGGAAGCTGGGAATTCTTCAAACT |
| | TGACAACTCTATCCGGAGTTGTTATCTCATATTCTGAAGGCATG |
| | AGTGATGATGCTCTGGAGAAGTTCCCAATTCTTCAGCACCTTG |
| | ATTGCATCATCATGGAATCGCAGAATGCTCCTACACACGACTA |
| | TTGGTTTCTCAAGCTTGATGTACTTAATAAACTCGAATCATTCG |
| | TAGCAAGATACAAGCGCAATGGACATCCCTCGTTAAACCGACA |
| | ACCGTATGGATTTCACTTCCCTACAAGCTTGAAAGAGTTACGG |
| | TTGACTGGTTTTTTCCTGAGACCTGATTTGTTGTCAGTAATCGC |
| | AGCGTTGCCTGAGCTTGAGATTATGGAGTTTTCCGGCTGTTATT |
| | TCGTGGATACTAAATGGGACGCAAGTGAGGACATCTATCTAAG |
| | TCTTAAGACTTTGATTTTGCGAGATGTCCATTTATCAGAATGGC |
| | AAGTTGAGGGGGGAACTTTTCCCAAGCTTGAGAAATTAATACT |
| | AAAATTTTGTTCCACACTTGGGGAGATCCCTTGTGCATTTATGG |
| | ATGTAGAAACTTTAAAGTCCATTGATTTAAGTTTTGTTGGGCGT |
| | AAGCTTCGAGATTCAGCCATTGAGATTAAGAAAAATGTAGCAG |
| | ATTTCACAGGAGAGGACAGAGTTGATGTCCACACATCACATTT |
| | GTTTGCAACCAACGTGAAAGAACAGATTATGAGAATGACGGT |
| | GGTTGATGAAGTTGGATTTCTCATGGAAAAATCACAACAATGA |
| SEQ ID NO: | ATGGAAAAAGGAGAAAAATCATTGCAGCTACTATTTGAGGAA |
| 9: | CAAAAAACAAGAATCACGAATCTTATTGATGATTTCTTGATTC |
| CDS MeR1 | GCTTGAAGCAAATAAAGAATGAATTCATTGCTTCAAAATTGGA |
| (isoform 8) | TGCCTTTGAAAATCTAAGAATGGAACTGAGATTCCTAAGAACA |
| | TTTGTCCTGTTTGGGAATTCGATGAATTTGGATGACTTTTATGA |
| | GAGGATGTCACTGAGTATAAGCAAATTCGATCAATCTACGGAG |
| | TACGTAGATAAAATAATCCTAGAGAAATACAACATGGAATGTC |
| | TTGCCCCTCTGCTGCTTGAAGAGATAAGAAATTATTTGAGTTTG |
| | AAGAATGATTATGTAGCCACAACTACAGAGATAAAATTGTTTG |
| | AATACCTCATCAGAAACCTCCATGATCTACCAAAGTATTGTTCT |
| | GATTTGCTTCTACCACTCATGAGTGAATACAAGATTCTTCGGCA |
| | AGTATGCACACATCTCAGAGATTTCTATCAGTTGGAATGCAAC |
| | AAAACAACAAAAACAGAATTTCTCTATACTCGGTATCAAGTGA |
| | CAGCCGATAGAGTAACACAATTCTGTTTTGATCTTTGGACAGG |
| | AATGTATAAATACTCTGACAATGAGTATGCCTTCTCTGAATGTT |
| | CTTCCAAGATCACTTCTCTACTCATTGACATAATCCCTCTTGAG |
| | TTGGAGGTTCTATACATTTCTACTTCTAAGCTCATCAAAGAGTC |
| | AACGTCAAAAGAACTAGAAGGATTTGTTAAGCAAATCTTAAAA |
| | GCATCTCCAAGGATTCTTCAACATTATCTCATTCATCTCCAAGG |
| | ACGCATGGCCGGTGTAGAAGCCGTCAATTTCGCTCCAACTCGA |
| | AGCATTAGTGTCATGATGGAGTTCCTATTGATCTTTCTCACCGA |
| | TATGCCAAAGCGGTTTATCCATCGTGAAAAATTGAACGATATG |
| | TTGGCACATGTCAGAATACTTACAAGGAAAATATCTACTCTGG |
| | TGAGTAAACTGTTGGAGGAGATCTCTGAGGATAATATCAATGA |
| | AGCGGACTTTTCAGCTCCGGACTTTTTGCAAGAAATTGAACAA |
| | ATGAAGGGAGATATCAGACACATCTTTTTAAAAGCTCCTGAGT |
| | CATCTCAACTTCGGTTTCCTATGGATGATGGTTTCCTCTTCATG |
| | AATCTTCTACTCAGACATTTAAATGATTTACTCATTTCCAATGC |
| | TTATTCAGTTTTTCTCATAAAAAAAGAAATTGGGATGGTGAAA |
| | GAAAGCCTTGAATTCCTAATATCATCTTTCAGGAAAGTCAGGC |
| | AAACATTGGATGAGAGTACTAGTGGAGTAGTTAAAAATTGTTG |
| | GGTGCGTGCTTTGGATGTGGCATATGAGGCAGAACATATCATT |
| | AATTCCATTCTTGTCAGAGATAAAGCTCTCTCACATCTCCTCTT |
| | CTCACTTCCGAGTGTCACTGATAAGATCAAACTTATCGTGGAA |
| | CAAGTCACCAGGTTTCAGCTGGAGGATAAGAATGGGGATGGC |
| | CCCCTTGATGCAAAGTCTTCCTTCGAGCCAACTCAGTCAACTTC |
| | ATCACCTTTTGTTGAGGTAACAGTAGGTCACGAGAAAGAAGAA |
| | TCCCAGATCATTGACCAGCTCCTTGATGAACATGAATCTGAGC |
| | TTGATGTCATTTCCATTGTCGGAATGCCAGGACTCGGTAAAAC |
| | TACTCTGGCCAACAAAGTGTATAAAGATACATTAGTTGCTAGG |
| | CATTTCCATGTCCGTGCTTGGTGCACTGTTTCGCAAAAGTATAA |
| | CAAGTCAAAGGTGTTGCGGGAGATTCTTCAGCAAGTTACTGGC |
| | TCGGGAGGAAAAGAAAGTGAAGATGACCTGGCTGAAAAGCTA |
| | CGAAGAGCACTACTCGATAAAAGGTACCTAATCGTCTTGGATG |
| | ATGTGTGGGATATTGCAACAGGGGAGATGTTAATAGCATGTTT |
| | TCCTAAGGGTAAGAGAGGAAATAGAATCATCTTAACTAGCCGA |
| | AGTAGAAAGGTAGGTTTGAAAGTTAAATGCCGTAGTGATCCTC |
| | TCGACCTTAAACTTTTAACATCTGAAGAAAGTTGGGATTTATTC |
| | GAAAAAAGGGTATTTGGAGATGAAGGAAGCTGCCCTGCTGAA |
| | CTGTCCGAAGTTGGACACCAAATAGTTGAGAAATGTAAGGGTC |
| | TTCCCTTGGCTATTGTTTTAATTGCTGGAGTAATTGTTAGAGGG |
| | AAGAAAAAGGAAAAGGATTTTTGGCTTAAGATACTGCATAATC |
| | TGGATTCCTTTATTTCTACCAACATCAATTTGGTTATGCAATTA |
| | AGTTATGACCATTTACCATGCCACCTGAAGCCGTTGCTGCTTTA |
| | CTTTGCAACAACTCAAAAGAGCCAACAAACTCCAGTCTCTACA |
| | TTGATGCAGTTGTGGATGGCCGAAGGGTTTGTGGATCATGATA |
| | GTTTAGAGGAAGTAACTCAAAGTTACTTGGATGCTCTAATTTC |
| | CAGTAGCCTGATAATGGTGGATCATATCCCCTCCAAGAGTTAT |
| | TGGTGGACGTCTTTAATGATCAAGGTTTGCTATGTGCATGATGT |
| | TGTGCACGATTTTTGTTCAGAAAAAGCCAAAAAGGAGAAGTTT |
| | CTCAAGTTAATCAATTCAGGTGATCCATTTCATGCTTCAGATTT |
| | CCTACACCATCGTCTAACCATTCATACTGACAACGGCCAACTC |
| | CACAAAAAATGTGTTTTATTCAATTCTAATAAGTGCTTAGCTGT |
| | TAGTAAGCATGTCATATCTTTGAAAGTGAGTGGTCCACTAGAT |
| | GAGTTCAGGTATATCTGTCACACAAGACACTTTGGACTTGTTA |
| | GAGTGTTGCAACTGGATGACATCATTCTGGAAGATTCTTTAAT |
| | GGAAGAAATAGGGTCCCTATTTCATTTGAGGTTCTTAAGCATT |
| | GAGACTGCTGTAGGTGTAATAGCTATCCCAGTGTCGTGGTTGA |
| | ACCTCCAGAATCTGGAAACGCTGTTGATTTATACAACTTATTCC |
| | ACCATGGTATTACTGCCCAGAATATTGCAACTGTCAAAGCTGA |
| | AACATGTGAAAATTAAGGAATGTTCTTTCTTTGAAGAGAAAGA |
| | GGATATCCAACGTAGAATATTGGAAGCTGGGAATTCTTCAAAC |
| | TTGACAACTCTATCCGGAGTTGTTATCTCATATTCTGAAGGCAT |
| | GAGTGATGATGCTCTGGAGAAGTTCCCAATTCTTCAGCACCTT |
| | GATTGCATCATCATGGAATCGCAGAATGCTCCTACACACGACT |
| | ATTGGTTTCTCAAGCTTGATGTACTTAATAAACTCGAATCATTC |
| | GTAGCAAGATACAAGCGCAATGGACATCCCTCGTTAAACCGAC |
| | AACCGTATGGATTTCACTTCCCTACAAGCTTGAAAGAGTTACG |
| | GTTGACTGGTTTTTTCCTGAGACCTGATTTGTTGTCAGTAATCG |
| | CAGCGTTGCCTGAGCTTGAGATTATGGAGTTTTCCGGCTGTTAT |
| | TTCGTGGATACTAAATGGGACGCAAGTGAGGACATCTATCTAA |
| | GTCTTAAGACTTTGATTTTGCGAGATGTCCATTTATCAGAATGG |
| | CAAGTTGAGGGGGGAACTTTTCCCAAGCTTGAGAAATTAATAC |
| | TAAAATTTTGTTCCACACTTGGGGAGATCCCTTGTGCATTTATG |
| | GATGTAGAAACTTTAAAGTCCATTGATTTAAGTTTTGTTGGGCG |
| | TAAGCTTCGAGATTCAGCCATTGAGATTAAGAAAAATGTAGCA |
| | GATTTCACAGGAGAGGACAGAGTTGATGTCCACACATCACATT |
| | TGTTTGCAACCAACGTGAAAGAACAGATTATGAGAATGACGGT |
| | GGTTGATGAAGTTGGATTTCTCATGGAAAAATCACAACAATGA |
| SEQ ID NO: | MEKGEKSLLLFEEQKTRITNLIDDFLIRLKQIKNEFIASKLDAFENL |
| 10: | RMELRFLRTFVLFGNSMNLDDFYERMSLSISKFDQSTEYVDKIILE |
| protein MeR1 | KYNMECLAPLLLEEIRNYLSLKNDYVATTTEIKLFEYLIRNLHDLP |
| (isoform 1) | KYCSDLLLPLMSEYKILRQVCTHLRDFYQLECNKTTKTEFLYTRY |
| | QVTADRVTQFCFDLWTGMYKYSDNEYAFSECSSKITSLLIDIIPLE |
| | LEVLYISTSKLIKESTSKELEGFVKQILKASPRILQHYLIHLQGRMA |
| | GVEAVNFAPTRSISVMMEFLLIFLTDMPKRFIHREKLNDMLAHVRI |
| | LTRKISTLVSKLLEEISEDNINEADFSAPDFLQEIEQMKGDIRHIFLK |
| | APESSQLRFPMDDGFLFMNLLLRHLNDLLISNAYSVFLIKKEIGMV |
| | KESLEFLISSFRKVRQTLDESTSGVVKNCWVRALDVAYEAEHIINS |
| | ILVRDKALSHLLFSLPSVTDKIKLIVEQVTRFQLEDKNGDGPLDAK |
| | SSFEPTQSTSSPFVEVTVGHEKEESQIIDQLLDEHESELDVISIVGMP |
| | GLGKTTLANKVYKDTLVARHFHVRAWCTVSQKYNKSKVLREIL |
| | QQVTGSGGKESEDDLAEKLRRALLDKRYLIVLDDVWDIATGEML |
| | IACFPKGKRGNRIILTSRSRKVGLKVKCRSDPLDLKLLTSEESWDL |
| | FEKRVFGDEGSCPAELSEVGHQIVEKCKGLPLAIVLIAGVIVRGKK |
| | KEKDFWLKILHNLDSFISTNINLVMQLSYDHLPCHLKPLLLYFATT |
| | QKSQQTPVSTLMQLWMAEGFVDHDSLEEVTQSYLDALISSSLIMV |
| | DHIPSKSYWWTSLMIKVCYVHDVVHDFCSEKAKKEKFLKLINSG |
| | DPFHASDFLHHRLTIHTDNGQLHKKCVLFNSNKCLAVSKHVISLK |
| | VSGPLDEFRYICHTRHFGLVRVLQLDDIILEDSLMEEIGSLFHLRFL |
| | SIETAVGVIAIPVSWLNLQNLETLLIYTTYSTMVLLPRILQLSKLKH |
| | VKIKECSFFEEKEDIQRRILEAGNSSNLTTLSGVVISYSEGMSDDAL |
| | EKFPILQHLDCIIMESQNAPTHDYWFLKLDVLNKLESFVARYKRN |
| | GHPSLNRQPYGFHFPTSLKELRLTGFFLRPDLLSVIAALPELEIMEF |
| | SGCYFVDTKWDASEDIYLSLKTLILRDVHLSEWQVEGGTFPKLEK |
| | LILKFCSTLGEIPCAFMDVETLKSIDLSFVGRKLRDSAIEIKKNVAD |
| | FTGEDRVDVHTSHLFATNVKEQIMRMTGMGNIQGRRGI |
| SEQ ID NO: | MEKGEKSLQLLFEEQKTRITNLIDDFLIRLKQIKNEFIASKLDAFEN |
| 11: | LRMELRFLRTFVLFGNSMNLDDFYERMSLSISKFDQSTEYVDKIIL |
| protein MeR1 | EKYNMECLAPLLLEEIRNYLSLKNDYVATTTEIKLFEYLIRNLHDL |
| (isoform 2) | PKYCSDLLLPLMSEYKILRQVCTHLRDFYQLECNKTTKTEFLYTR |
| | YQVTADRVTQFCFDLWTGMYKYSDNEYAFSECSSKITSLLIDIIPL |
| | ELEVLYISTSKLIKESTSKELEGFVKQILKASPRILQHYLIHLQGRM |
| | AGVEAVNFAPTRSISVMMEFLLIFLTDMPKRFIHREKLNDMLAHV |
| | RILTRKISTLVSKLLEEISEDNINEADFSAPDFLQEIEQMKGDIRHIF |
| | LKAPESSQLRFPMDDGFLFMNLLLRHLNDLLISNAYSVFLIKKEIG |
| | MVKESLEFLISSFRKVRQTLDESTSGVVKNCWVRALDVAYEAEHI |
| | INSILVRDKALSHLLFSLPSVTDKIKLIVEQVTRFQLEDKNGDGPLD |
| | AKSSFEPTQSTSSPFVEVTVGHEKEESQIIDQLLDEHESELDVISIVG |
| | MPGLGKTTLANKVYKDTLVARHFHVRAWCTVSQKYNKSKVLRE |
| | ILQQVTGSGGKESEDDLAEKLRRALLDKRYLIVLDDVWDIATGE |
| | MLIACFPKGKRGNRIILTSRSRKVGLKVKCRSDPLDLKLLTSEESW |
| | DLFEKRVFGDEGSCPAELSEVGHQIVEKCKGLPLAIVLIAGVIVRG |
| | KKKEKDFWLKILHNLDSFISTNINLVMQLSYDHLPCHLKPLLLYF |
| | ATTQKSQQTPVSTLMQLWMAEGFVDHDSLEEVTQSYLDALISSSL |
| | IMVDHIPSKSYWWTSLMIKVCYVHDVVHDFCSEKAKKEKFLKLI |
| | NSGDPFHASDFLHHRLTIHTDNGQLHKKCVLFNSNKCLAVSKHVI |
| | SLKVSGPLDEFRYICHTRHFGLVRVLQLDDIILEDSLMEEIGSLFHL |
| | RFLSIETAVGVIAIPVSWLNLQNLETLLIYTTYSTMVLLPRILQLSK |
| | LKHVKIKECSFFEEKEDIQRRILEAGNSSNLTTLSGVVISYSEGMSD |
| | DALEKFPILQHLDCIIMESQNAPTHDYWFLKLDVLNKLESFVARY |
| | KRNGHPSLNRQPYGFHFPTSLKELRLTGFFLRPDLLSVIAALPELEI |
| | MEFSGCYFVDTKWDASEDIYLSLKTLILRDVHLSEWQVEGGTFPK |
| | LEKLILKFCSTLGEIPCAFMDVETLKSIDLSFVGRKLRDSAIEIKKN |
| | VADFTGEDRVDVHTSHLFATNVKEQIMRMTGMGNIQGRRGI |
| SEQ ID NO: | MEKGEKSLLLFEEQKTRITNLIDDFLIRLKQIKNEFIASKLDAFENL |
| 12: | RMELRFLRTFVLFGNSMNLDDFYERMSLSISKFDQSTEYVDKIILE |
| protein MeR1 | KYNMECLAPLLLEEIRNYLSLKNDYVATTTEIKLFEYLIRNLHDLP |
| (isoform 3) | KYCSDLLLPLMSEYKILRQVCTHLRDFYQLECNKTTKTEFLYTRY |
| | QVTADRVTQFCFDLWTGMYKYSDNEYAFSECSSKITSLLIDIIPLE |
| | LEVLYISTSKLIKESTSKELEGFVKQILKASPRILQHYLIHLQGRMA |
| | GVEAVNFAPTRSISVMMEFLLIFLTDMPKRFIHREKLNDMLAHVRI |
| | LTRKISTLVSKLLEEISEDNINEADFSAPDFLQEIEQMKGDIRHIFLK |
| | APESSQLRFPMDDGFLFMNLLLRHLNDLLISNAYSVFLIKKEIGMV |
| | KESLEFLISSFRKVRQTLDESTSGVVKNCWVRALDVAYEAEHIINS |
| | ILVRDKALSHLLFSLPSVTDKIKLIVEQVTRFQLEDKNGDGPLDAK |
| | SSFEPTQSTSSPFVEVTVGHEKEESQIIDQLLDEHESELDVISIVGMP |
| | GLGKTTLANKVYKDTLVARHFHVRAWCTVSQKYNKSKVLREIL |
| | QQVTGSGGKESEDDLAEKLRRALLDKRYLIVLDDVWDIATGEML |
| | IACFPKGKRGNRIILTSRSRKVGLKVKCRSDPLDLKLLTSEESWDL |
| | FEKRVFGDEGSCPAELSEVGHQIVEKCKGLPLAIVLIAGVIVRGKK |
| | KEKDFWLKILHNLDSFISTNINLVMQLSYDHLPCHLKPLLLYFATT |
| | QKSQQTPVSTLMQLWMAEGFVDHDSLEEVTQSYLDALISSSLIMV |
| | DHIPSKSYWWTSLMIKVCYVHDVVHDFCSEKAKKEKFLKLINSG |
| | DPFHASDFLHHRLTIHTDNGQLHKKCVLFNSNKCLAVSKHVISLK |
| | VSGPLDEFRYICHTRHFGLVRVLQLDDIILEDSLMEEIGSLFHLRFL |
| | SIETAVGVIAIPVSWLNLQNLETLLIYTTYSTMVLLPRILQLSKLKH |
| | VKIKECSFFEEKEDIQRRILEAGNSSNLTTLSGVVISYSEGMSDDAL |
| | EKFPILQHLDCIIMESQNAPTHDYWFLKLDVLNKLESFVARYKRN |
| | GHPSLNRQPYGFHFPTSLKELRLTGFFLRPDLLSVIAALPELEIMEF |
| | SGCYFVDTKWDASEDIYLSLKTLILRDVHLSEWQVEGGTFPKLEK |
| | LILKFCSTLGEIPCAFMDVETLKSIDLSFVGRKLRDSAIEIKKNVAD |
| | FTGEDRVDVHTSHLFATNVKEQIMRMTGGECTCEEL |
| SEQ ID NO: | MEKGEKSLQLLFEEQKTRITNLIDDFLIRLKQIKNEFIASKLDAFEN |
| 13: | LRMELRFLRTFVLFGNSMNLDDFYERMSLSISKFDQSTEYVDKIIL |
| protein MeR1 | EKYNMECLAPLLLEEIRNYLSLKNDYVATTTEIKLFEYLIRNLHDL |
| (isoform 4) | PKYCSDLLLPLMSEYKILRQVCTHLRDFYQLECNKTTKTEFLYTR |
| | YQVTADRVTQFCFDLWTGMYKYSDNEYAFSECSSKITSLLIDIIPL |
| | ELEVLYISTSKLIKESTSKELEGFVKQILKASPRILQHYLIHLQGRM |
| | AGVEAVNFAPTRSISVMMEFLLIFLTDMPKRFIHREKLNDMLAHV |
| | RILTRKISTLVSKLLEEISEDNINEADFSAPDFLQEIEQMKGDIRHIF |
| | LKAPESSQLRFPMDDGFLFMNLLLRHLNDLLISNAYSVFLIKKEIG |
| | MVKESLEFLISSFRKVRQTLDESTSGVVKNCWVRALDVAYEAEHI |
| | INSILVRDKALSHLLFSLPSVTDKIKLIVEQVTRFQLEDKNGDGPLD |
| | AKSSFEPTQSTSSPFVEVTVGHEKEESQIIDQLLDEHESELDVISIVG |
| | MPGLGKTTLANKVYKDTLVARHFHVRAWCTVSQKYNKSKVLRE |
| | ILQQVTGSGGKESEDDLAEKLRRALLDKRYLIVLDDVWDIATGE |
| | MLIACFPKGKRGNRIILTSRSRKVGLKVKCRSDPLDLKLLTSEESW |
| | DLFEKRVFGDEGSCPAELSEVGHQIVEKCKGLPLAIVLIAGVIVRG |
| | KKKEKDFWLKILHNLDSFISTNINLVMQLSYDHLPCHLKPLLLYF |
| | ATTQKSQQTPVSTLMQLWMAEGFVDHDSLEEVTQSYLDALISSSL |
| | IMVDHIPSKSYWWTSLMIKVCYVHDVVHDFCSEKAKKEKFLKLI |
| | NSGDPFHASDFLHHRLTIHTDNGQLHKKCVLFNSNKCLAVSKHVI |
| | SLKVSGPLDEFRYICHTRHFGLVRVLQLDDIILEDSLMEEIGSLFHL |
| | RFLSIETAVGVIAIPVSWLNLQNLETLLIYTTYSTMVLLPRILQLSK |
| | LKHVKIKECSFFEEKEDIQRRILEAGNSSNLTTLSGVVISYSEGMSD |
| | DALEKFPILQHLDCHIMESQNAPTHDYWFLKLDVLNKLESFVARY |
| | KRNGHPSLNRQPYGFHFPTSLKELRLTGFFLRPDLLSVIAALPELEI |
| | MEFSGCYFVDTKWDASEDIYLSLKTLILRDVHLSEWQVEGGTFPK |
| | LEKLILKFCSTLGEIPCAFMDVETLKSIDLSFVGRKLRDSAIEIKKN |
| | VADFTGEDRVDVHTSHLFATNVKEQIMRMTGGECTCEEL |
| SEQ ID NO: | MEKGEKSLLLFEEQKTRITNLIDDFLIRLKQIKNEFIASKLDAFENL |
| 14: | RMELRFLRTFVLFGNSMNLDDFYERMSLSISKFDQSTEYVDKIILE |
| protein MeR1 | KYNMECLAPLLLEEIRNYLSLKNDYVATTTEIKLFEYLIRNLHDLP |
| (isoform 5) | KYCSDLLLPLMSEYKILRQVCTHLRDFYQLECNKTTKTEFLYTRY |
| | QVTADRVTQFCFDLWTGMYKYSDNEYAFSECSSKITSLLIDIIPLE |
| | LEVLYISTSKLIKESTSKELEGFVKQILKASPRILQHYLIHLQGRMA |
| | GVEAVNFAPTRSISVMMEFLLIFLTDMPKRFIHREKLNDMLAHVRI |
| | LTRKISTLVSKLLEEISEDNINEADFSAPDFLQEIEQMKGDIRHIFLK |
| | APESSQLRFPMDDGFLFMNLLLRHLNDLLISNAYSVFLIKKEIGMV |
| | KESLEFLISSFRKVRQTLDESTSGVVKNCWVRALDVAYEAEHIINS |
| | ILVRDKALSHLLFSLPSVTDKIKLIVEQVTRFQLEDKNGDGPLDAK |
| | SSFEPTQSTSSPFVEVTVGHEKEESQIIDQLLDEHESELDVISIVGMP |
| | GLGKTTLANKVYKDTLVARHFHVRAWCTVSQKYNKSKVLREIL |
| | QQVTGSGGKESEDDLAEKLRRALLDKRWRMYL |
| SEQ ID NO: | MEKGEKSLQLLFEEQKTRITNLIDDFLIRLKQIKNEFIASKLDAFEN |
| 15: | LRMELRFLRTFVLFGNSMNLDDFYERMSLSISKFDQSTEYVDKIIL |
| protein MeR1 | EKYNMECLAPLLLEEIRNYLSLKNDYVATTTEIKLFEYLIRNLHDL |
| (isoform 6) | PKYCSDLLLPLMSEYKILRQVCTHLRDFYQLECNKTTKTEFLYTR |
| | YQVTADRVTQFCFDLWTGMYKYSDNEYAFSECSSKITSLLIDIIPL |
| | ELEVLYISTSKLIKESTSKELEGFVKQILKASPRILQHYLIHLQGRM |
| | AGVEAVNFAPTRSISVMMEFLLIFLTDMPKRFIHREKLNDMLAHV |
| | RILTRKISTLVSKLLEEISEDNINEADFSAPDFLQEIEQMKGDIRHIF |
| | LKAPESSQLRFPMDDGFLFMNLLLRHLNDLLISNAYSVFLIKKEIG |
| | MVKESLEFLISSFRKVRQTLDESTSGVVKNCWVRALDVAYEAEHI |
| | INSILVRDKALSHLLFSLPSVTDKIKLIVEQVTRFQLEDKNGDGPLD |
| | AKSSFEPTQSTSSPFVEVTVGHEKEESQIIDQLLDEHESELDVISIVG |
| | MPGLGKTTLANKVYKDTLVARHFHVRAWCTVSQKYNKSKVLRE |
| | ILQQVTGSGGKESEDDLAEKLRRALLDKRWRMYL |
| SEQ ID NO: | MEKGEKSLLLFEEQKTRITNLIDDFLIRLKQIKNEFIASKLDAFENL |
| 16: | RMELRFLRTFVLFGNSMNLDDFYERMSLSISKFDQSTEYVDKIILE |
| protein MeR1 | KYNMECLAPLLLEEIRNYLSLKNDYVATTTEIKLFEYLIRNLHDLP |
| (isoform 7) | KYCSDLLLPLMSEYKILRQVCTHLRDFYQLECNKTTKTEFLYTRY |
| | QVTADRVTQFCFDLWTGMYKYSDNEYAFSECSSKITSLLIDIIPLE |
| | LEVLYISTSKLIKESTSKELEGFVKQILKASPRILQHYLIHLQGRMA |
| | GVEAVNFAPTRSISVMMEFLLIFLTDMPKRFIHREKLNDMLAHVRI |
| | LTRKISTLVSKLLEEISEDNINEADFSAPDFLQEIEQMKGDIRHIFLK |
| | APESSQLRFPMDDGFLFMNLLLRHLNDLLISNAYSVFLIKKEIGMV |
| | KESLEFLISSFRKVRQTLDESTSGVVKNCWVRALDVAYEAEHIINS |
| | ILVRDKALSHLLFSLPSVTDKIKLIVEQVTRFQLEDKNGDGPLDAK |
| | SSFEPTQSTSSPFVEVTVGHEKEESQIIDQLLDEHESELDVISIVGMP |
| | GLGKTTLANKVYKDTLVARHFHVRAWCTVSQKYNKSKVLREIL |
| | QQVTGSGGKESEDDLAEKLRRALLDKRYLIVLDDVWDIATGEML |
| | IACFPKGKRGNRIILTSRSRKVGLKVKCRSDPLDLKLLTSEESWDL |
| | FEKRVFGDEGSCPAELSEVGHQIVEKCKGLPLAIVLIAGVIVRGKK |
| | KEKDFWLKILHNLDSFISTNINLVMQLSYDHLPCHLKPLLLYFATT |
| | QKSQQTPVSTLMQLWMAEGFVDHDSLEEVTQSYLDALISSSLIMV |
| | DHIPSKSYWWTSLMIKVCYVHDVVHDFCSEKAKKEKFLKLINSG |
| | DPFHASDFLHHRLTIHTDNGQLHKKCVLFNSNKCLAVSKHVISLK |
| | VSGPLDEFRYICHTRHFGLVRVLQLDDIILEDSLMEEIGSLFHLRFL |
| | SIETAVGVIAIPVSWLNLQNLETLLIYTTYSTMVLLPRILQLSKLKH |
| | VKIKECSFFEEKEDIQRRILEAGNSSNLTTLSGVVISYSEGMSDDAL |
| | EKFPILQHLDCIIMESQNAPTHDYWFLKLDVLNKLESFVARYKRN |
| | GHPSLNRQPYGFHFPTSLKELRLTGFFLRPDLLSVIAALPELEIMEF |
| | SGCYFVDTKWDASEDIYLSLKTLILRDVHLSEWQVEGGTFPKLEK |
| | LILKFCSTLGEIPCAFMDVETLKSIDLSFVGRKLRDSAIEIKKNVAD |
| | FTGEDRVDVHTSHLFATNVKEQIMRMTVVDEVGFLMEKSQQ |
| SEQ ID NO: | MEKGEKSLQLLFEEQKTRITNLIDDFLIRLKQIKNEFIASKLDAFEN |
| 17: | LRMELRFLRTFVLFGNSMNLDDFYERMSLSISKFDQSTEYVDKIIL |
| protein MeR1 | EKYNMECLAPLLLEEIRNYLSLKNDYVATTTEIKLFEYLIRNLHDL |
| (isoform 8) | PKYCSDLLLPLMSEYKILRQVCTHLRDFYQLECNKTTKTEFLYTR |
| | YQVTADRVTQFCFDLWTGMYKYSDNEYAFSECSSKITSLLIDIIPL |
| | ELEVLYISTSKLIKESTSKELEGFVKQILKASPRILQHYLIHLQGRM |
| | AGVEAVNFAPTRSISVMMEFLLIFLTDMPKRFIHREKLNDMLAHV |
| | RILTRKISTLVSKLLEEISEDNINEADFSAPDFLQEIEQMKGDIRHIF |
| | LKAPESSQLRFPMDDGFLFMNLLLRHLNDLLISNAYSVFLIKKEIG |
| | MVKESLEFLISSFRKVRQTLDESTSGVVKNCWVRALDVAYEAEHI |
| | INSILVRDKALSHLLFSLPSVTDKIKLIVEQVTRFQLEDKNGDGPLD |
| | AKSSFEPTQSTSSPFVEVTVGHEKEESQIIDQLLDEHESELDVISIVG |
| | MPGLGKTTLANKVYKDTLVARHFHVRAWCTVSQKYNKSKVLRE |
| | ILQQVTGSGGKESEDDLAEKLRRALLDKRYLIVLDDVWDIATGE |
| | MLIACFPKGKRGNRIILTSRSRKVGLKVKCRSDPLDLKLLTSEESW |
| | DLFEKRVFGDEGSCPAELSEVGHQIVEKCKGLPLAIVLIAGVIVRG |
| | KKKEKDFWLKILHNLDSFISTNINLVMQLSYDHLPCHLKPLLLYF |
| | ATTQKSQQTPVSTLMQLWMAEGFVDHDSLEEVTQSYLDALISSSL |
| | IMVDHIPSKSYWWTSLMIKVCYVHDVVHDFCSEKAKKEKFLKLI |
| | NSGDPFHASDFLHHRLTIHTDNGQLHKKCVLFNSNKCLAVSKHVI |
| | SLKVSGPLDEFRYICHTRHFGLVRVLQLDDIILEDSLMEEIGSLFHL |
| | RFLSIETAVGVIAIPVSWLNLQNLETLLIYTTYSTMVLLPRILQLSK |
| | LKHVKIKECSFFEEKEDIQRRILEAGNSSNLTTLSGVVISYSEGMSD |
| | DALEKFPILQHLDCIIMESQNAPTHDYWFLKLDVLNKLESFVARY |
| | KRNGHPSLNRQPYGFHFPTSLKELRLTGFFLRPDLLSVIAALPELEI |
| | MEFSGCYFVDTKWDASEDIYLSLKTLILRDVHLSEWQVEGGTFPK |
| | LEKLILKFCSTLGEIPCAFMDVETLKSIDLSFVGRKLRDSAIEIKKN |
| | VADFTGEDRVDVHTSHLFATNVKEQIMRMTVVDEVGFLMEKSQ |
| | Q |
| SEQ ID NO: | ATTCAATTTTAAAAAATTATTCAGTAAAATTATTAAATTTTATT |
| 18: | TTGTATCGTTAAAATTACTCAATTTTGATATATTTAAATCAGTG |
| Genomic | AAATTATTAAACTAAATTTATCATTAAAATAATAAAATAATTTT |
| NRC6-2a | TTAAATATATAATTACAAATAAAATAATAAATAAATACTTAAA |
| | ATTACTACTTTATTATATATTGTAAGATAAAATAATAAAAGAT |
| | AATTTTATCAATTTCATTTGGTACACTGCTTCATTATTGCAATG |
| | ACGACTGTCGATTTTGCTCCGGTAGTTTAATCAATTTGTAGTGG |
| | TTTAATCAATTTTTTGTTTGTGTGAATCCCTTATTTAATTTTTTT |
| | TTAAAAAAAATAAATAAAGTGAGGTTTCATGTATCAATGGTAG |
| | TTTGGACAATTTTGAGTAGTTTGATAAATTTATTTTTTGTTTTGA |
| | ATTTTTATTATTTTATAATGAAATAGTAATTTCGAGTCATGTAC |
| | TATGTAGTTTAATCATAAACATAGATTGAGTGATTTTAATGATA |
| | TAAAAACCTAAATTGAGTATTTTACTGGTACAAAATGAAGTTT |
| | AGTTGCTAGATATATTTCAAAAGTTGAGTGACCTTTTGAGAAA |
| | TTAACTCCTCTGTCAGATCTCCTAATTTGATAAATTTTCAAAAC |
| | TTCATAATATAACATTTTCCTTTTTTATAATGATTACAAACTAT |
| | ATCAAACGACGAGTTCCCCCTGTCACTTATACTTTTAATTCACA |
| | AATTCCTCTTATCAGTGTGACCACATCCCCACAACCATACTTGT |
| | TACGATCCAAAAATGAGCGTGATGACACTCGTCTTATTCACAT |
| | AGATTAGTCAGCCTAAAACTCAATTATTATGAAGAAGAGAGGA |
| | AATAAACTATAAAGTGGAATCAGATTCATGAGTTGTGTTTGAG |
| | TCTGAATTATTGAATATTAAAGTCTGATCAATTAAAGTCGAGA |
| | GTAATTAGTCTGTATAATTTGTTCTATGTTTATTACTGAGTTGT |
| | AATAATTTCTATATTTTGTAACCAAAAACGGATTTGACTTTTTG |
| | AAGAGAGTTTGAGAGAATTGTAACAAGAAATGTGTTTGACTTA |
| | TCGCAGAGTAGAGTTAAGAGAGAGTTAGAGAGAATTGTAATA |
| | AGAAATGAGTTTGACTTCTTAGTGAGTAGAGAGTTGAGAGAGA |
| | GTTTGAGAGAATTATAACAAGAAGTTTGACTTCTTGGAGAATA |
| | GAGTTTCCGATTATAATTAATCGTTGAAGAGTAAAGATACTAG |
| | ACTTAGTCCCTTTGATGATTGAATTGTTATTGAAGTTAATAAAA |
| | TTGTGATGTGATTTTTTCCTTCCTTGAGGGAGGAAGGCTTCCAC |
| | GGTAAATTAAGTGTTGTTTAAATTCTGTTCTAAACGACTATAGG |
| | AACCAGGTTCCTAGAAATAATTGTTGGCGCATAGTATCTTTCA |
| | AGGAAAAATGCAGAATGCCACGTAAGCTCGAAAAGGGTAGAA |
| | AATTATCTATAAAATAAGTTCGGGTGGAAGGATAATAAGAGTT |
| | GTATAAGTGTGTCTCTGAAATTTGAGACATAGATTAGGAGGTA |
| | CTTATGTATTTTCCCTTCTTCCAGTTATTTGTTTCACCTCTTCCA |
| | TAATGTCAAATCCTTAATTATATATGTCTACCTCTGTACGTCAT |
| | ATTATAGATAGAGAAACAATTTAAATGTGTCTTTATAATCAAT |
| | ATCACTGAAAGCTCTAGCGACGTTAAATACAACAATATTATTT |
| | TTTTATTTTGCTACCTTGAATAATAATTCTCAATTGTTTACAAG |
| | CTTTCCAACAACACCCATTTATTTCAACCAATGTGAACCTCTTT |
| | CCAACAACACTTAATTTGTTTTCTAGTAATTTTTCAATCAACAT |
| | GAACTTAATTATTCAAATGTTAGCTGTCATTGGATTATTAAATT |
| | GTTAATGTTGTAAGGTGATGGTGTATTATATTAAGAATTAACA |
| | AATAATACTTAAAGACTTGGTTAATCTAATTCAGAGTCAACTT |
| | GAAGTTATTTCATTCCACATGTTTATAAACACACAACTATGGC |
| | AATAACAACATAGCAATTGTCATCTCTAAATTACTACTACTATT |
| | CTTCTTCTCCATCAATTTAATCCTCAGAAATGGATCCGTTTACG |
| | GCGGCTGCATTAACCACTGCGGTGACAGCCACGGTGAGCCTTC |
| | TGGTGGAGAACTTGTCGCATCTTATAAGTTATAATTGGAAGTT |
| | GTATACAGGATTGAAGAAATCATGCGAAGATTTGTATGATGAA |
| | GTGAAGCGATTAAATGCATTTTTAGTCGATAACGCGAATCAGA |
| | GAAGTAATAGTACGCAATGGGATGTATTAGTCGATAAAATTCG |
| | ACGTACAGTATATAAAGCAGAGGATGTTGTTGATAAATTATTG |
| | ATTCAGGGCAAGTTAGACCAAGAGAGTAATATAGCTAAAAAG |
| | ATGTTTCACAAAACTTACAAAAACAGGAATTTTACTGAGGAAA |
| | TCAATGAGATACTTGTAGAGGTGAGGAAAATCCTTGAGGAAA |
| | ATCAACATCTGTTTGAGGCAAACCCGACGATTGATCATCATCA |
| | GCCTGAAAAAGTTGTCCAGGAGGAACAGGTACGTTTACACGA |
| | AATTTCGATTTTAATGTGATTTATTGAAATATGAAATGAACTAT |
| | GATTTGCTCGTTCAGTAAAACTGTATAAGAGTTGGAGAAATAG |
| | TACTTCCTCTGTTTTAATATGCATTATTTGTTTGAACTTAGTTAA |
| | AGTTTAATCTAAATTATTCAGATCTAGGACAAGGCATTAGGCA |
| | TAAGTAAGGCAAAGGGTAGAAGTACCTTTCTAGATTATGACTC |
| | AAATTCCGTAGAATTTTACTTTAGTTAAGGTGTGTCTCTGAAAT |
| | TCAGACAATTAAAGGTCACCACCAACCATTATTACATGCACAA |
| | ATCACTATCGATAATTGCCACCATTGGTCAACATTATATATCGC |
| | TGACCTCCATCTATCATTAACTGTCAGTATGATTCCACTATTAA |
| | TTATCAAATGGTCATCAACAACAACCACAATTAATCACTAAGG |
| | CCAATCACTATACTGTCCACAACCACTATCATCTGTCAACACTA |
| | TCTAAAATCGATACACACAATCACTATCATTAGTCATTACCAC |
| | CACCGATTGTCACATTTTCTTGTAGTGAATGATGGGGATTGAA |
| | AATATGAAGAATAAAATAGACATGCATAGGATAAGGGGACAT |
| | ATTTTTCATAGTTGAATGGGACATTGAAAATTAATAACTTTCAC |
| | ATGATCATAACTCATTTGGTACCTGATGGAATAATTTCTTGTTA |
| | TGATGATACAATCAAGACAAGTAGCCTTAATAACTTGTTTAAT |
| | TTGTCCATCTTACAGGGTTCGTCATTGGAAAATCACGAAGTGG |
| | TTGGATTTGATGAAGAAGCAACGAAAGTGATCAATCGTCTGGT |
| | TGAAGGAGCAGAGTGTCTAGATGTTATCCCGGTTGTAGGAATG |
| | CCGGGACTTGGTAAAACCACACTGGCAAGAAAAATCTTTAATG |
| | ATCCTAAGATTTCGCGAGAATTTTTCAGCTACATTTGGGTTTTC |
| | ATCGGACAATCAACGTGTGTAAAAAGGGATATCCTTTTTAATA |
| | TTCTGAAAGGGTTCACAAATTCATTTGATGAATTCAAAAACAG |
| | AAATGAGGCAAGCATAACTGATGAAATACGTAAGCGTGTGGC |
| | TAATGGAGGTAAATGTCTCATTGTCTTGGATGATGTGTGGGAT |
| | ACAAATGTTGTAGATATTGTCAAGACAGTTTTCCCTGATAACA |
| | AAAAAGCCCACAGGATCATGATGACCACTCGACACGAAGACA |
| | TTGCTAGATCTGTCAATAAATATCCCCACAATCTGAAATTTCTG |
| | GATGGAGACGAAAGTTTCCAGCTGCTAGAAAAGAGAGCTTTTG |
| | GCGTTAGCCGTTGTCCTGTTGAGTTAGTAGAACATGGAGAAGC |
| | CATTGTAGCAAAATGTAGTGGAGTACCACTTACAATTGTGGTA |
| | ATTGCAGGAGCTTTAAGAGGTCGTACGAGTGAAATTGATTGGA |
| | AAGTAGTTAGGGAAAATGTGGGGAAGCATCTTATACAAGAAG |
| | ACAAACTTCAGAGATGTGTGAATGTTGTGAGATTGAGTTACAA |
| | TCATTTGCCTCAAGAAAAAAAATCTTGCTTCTTGTATTTTGGTG |
| | CCTTTCCTCAAGGATTTGATATCCCCGCTTGGAAATTGATTCGA |
| | CTCTGGATTGCTGAGGGACTCATAATGTCCAAGTTGTCGGGCA |
| | ACGAAATTGAAGAGATAGCAGAGTATTATCTTAATGACTTTGC |
| | CAACAGGAACTTAGTGATGGTGATGAAAAAGAAATCTAATGA |
| | TCGAATAAAAACATGTCGTGTTCACGACATGTTACATGAGTTT |
| | TGCGTTGAAGAGGCTACTAGATTGACTCTTTTCAAACAAGTAT |
| | GTCTCACATCTGATCAAGACATACAGAACTCAATTACTTGTCG |
| | TCGTGTCTCTATTCAATCATCTGTTCCTCAAAACTTCATCTCAA |
| | AAAAGACAGTTGAAGAACATGTTAGGTCATTGTTATGTTTTTC |
| | CTCAAAACAAAAACAAGTTGACTTTTCTAATATCGACGTCAAG |
| | CTCATCCCTACCGCATTTCCACTTATGAGAGTCTTAGACATTGA |
| | ATCCGTCAAGTTTAGTATTCCCAGGGAATTTTACCAGCTATTGC |
| | ACTTGAGGTATATCGCTATGTCAGGTGATTTCGAGCAACTTCCT |
| | AAACTCTTTACTTCTTTCTGTAATGCACAAACTCTCATTCTAAA |
| | TACTTCCAAGCCCACCCTTGATATAAAAGCTGACATATGGAAC |
| | ATGCCACGTTTGCGTCATCTGCACACCAACAAACCTGCAATCT |
| | TACCACCCCCTACAAGTAGTAGTAGTAGTAGTACAAATTCTTG |
| | TTTGTTGCAAACTCTATCTCTGGTTACACCAGAAAGCTGCAAG |
| | GGAAACGTTCTTTCAAAGGCTCGTAATGTCAAGAAAATGAGTG |
| | TTAAAGGTAATTTGACACCTTTTCTTGAAACTAGCAAGGGTGA |
| | ATTTTTCAGCAATTTTCAAGTGCTAAAGCTCCTGGAAAGTTTAA |
| | CACTGCTAAATGATGATAAGAGTAATAAATCTCTTCACCTTCC |
| | GTCAGCATTCTCCGAATGTTTACCAAATTTGAAGAAGTTAACT |
| | CTATCAAAAACAAGGTTTGACTGGAATCAGGCATATAGATTAG |
| | GGCAGGTGAAAAATCTCCAGGTCCTAAAACTGAAAGAAAATG |
| | CATTCACGGGGCCGTCCTGGAGGATGGAGCCAGGAGGTTTCAA |
| | GAAACTTCAGGTCTTGTGGATTGAAATGGCAGATTTCGTGTCG |
| | TGGGAGGCATCAAACTGTCCTTTCCCAAGACTTAGGAGCCTTT |
| | TCCTGATCTCCTGTCTTAATCTTGAGGCTGTGCCACTCGATCTT |
| | TCCCATTTGGATAACCTTCAAGAGATGACGTTGGAAAACACAA |
| | GCAAAGCAAGCAAATCTGCAAGAGAAATAGAATGCGAGAAAA |
| | AGAAGAAGCAAGCTGATGATCCAGAAACTGGCAAATTCAAGC |
| | TCACTATTCCCTACTGAAGCTGATTTCAATGCCACAAAGTGAA |
| | GTTGTTATAAGGTTAGTCCAATTCTCTCCCTCTCGTATCAGTAA |
| | ATATCGAATAAGATATTTTTCAATAAGATTGCATCTATCCCTAT |
| | AGTCTCTCCACGCAGAATTAGTAGGTAGCAGTGTGTTGTCTTG |
| | CTGTCTCGTTCATATCAATAGTCATAGTATTGCTCCTATAGTTT |
| | CTTATCCTTCGATTTTTCGTTACTATACGTTGTCCCTTATACCTC |
| | GATATTAGTAATGCTTGTTGTAGTTTCTCTTATGTTACTATCTG |
| | CTGTTTTTGTTATTACCTGTTGTTTCTTGTATTTCCGTCATTTCTT |
| | TTTGGGACTCCTTTGAACTTTCTCCTTGAGACAAAGGTCAATCG |
| | GAAACAACCTACCTCTCTTTCTTTGAGATAAGGTTCCTCCCCGA |
| | CTCTATTTTGTGGGACTACACTGAGTATATTGTTTTGGGTTTCG |
| | CACTTCACTGAAACATTAAATTGGGCAACATTACTCTGTTATAC |
| | TAATAATACAGATTTTAGGTGCCTTGATATCAAACATAGAGTA |
| | AATTCAATTATTTTCGTGTTCATCACATTCCTCATCTATTTTTCT |
| | CTTACTTATTTTCCTCCTTTTTAGCAGGAGAGTATCGAGGCATG |
| | GTGATGCATTTGCAGTCTCAGTTTTGATTACCTGTTCCAATAAA |
| | GAAACTTGTTACAAGTATGTTTTGTTGATTCAAACATGCCAAGT |
| | TTGAAAACGAAGTTTTATATTAATGTTTTTGCGTTTTCTTTCAG |
| | TGCTGAATAATTTGTCTAATCTGTAGTTGATGTTTGGATATCTA |
| | TGTATTGTTCGATTGACACGAGTTGTTTATACTATCATTTGTTG |
| | TTTATGAATATGTAAAACATATTTGATTTATGTTTGCAACGTCA |
| | ACGTGAACATTCAATTAGTCTAATCAAAGTCTAGATCTAAAAT |
| | TGATTAATTTAAGTTCATAATTTATTTTGAATTATTATCTAAAA |
| | CCGAAGACTTTCTACTCGAAAATCGAAATCATAGCTGACAGTC |
| | TCCATAAATATATTTGCAATTGAATAACATAATAAGCATTTATC |
| | AGTTAATTTTATGGAGTTTCTGGATTTACGTGATATTCAAACAT |
| | CTCCCGCGCATATTAATTATGTGAAGTAATGAAGTGTGTAACA |
| | TAAGTCAAAAGGTGCATAAATAAAGAGTCAAGGGTAATAAGA |
| | CTTTAGTTTACTTTAGGTGTGTCTCTGAAATTTCGATTATAATC |
| | TACGATATATTGTGTCTTGTCCCAACATTTAAATAAAATGAAC |
| | AAATAATTTTAATTACCAAATGGATCCATAGTCTGTTTAGTTGA |
| | CTAATGGAAAGATTTATAGGAAGGTGAGATTGTTTTATCCCTG |
| | ATTAGAGGTTTTGGTTCGAGTTTTGTGTATACGAAAAAATTGTG |
| | TTGAAAGCGTCACTTATGAATAGGCCCTATAATGTATGATTCG |
| | AATTTAGTCTGAGTTCTAATATGAATTTTGAACACATTGAAAA |
| | ATTATATCCTTTGTTAAATTTCATGGTTTTAACATCTTGGAAGT |
| | AAAGAAAAAGGTAAAATATTTTTTTTTTCTATTTCATTTTTTTA |
| | CATGTAATATTTGAATTCAAAGGCTGAGAGAGTAATACCCCCC |
| | TTTTATTTTTATTTGTGGATTACATATATACATAAGAACTAGAA |
| | AAATGTTTATATTTTCGACTTGACAATGATAGAATGTAAATGTT |
| | GGATATATAGAATAATTTGTTGGAGCTTCTACCATTTTTTGCTT |
| | ACCGTCTTAAGGTTGAAGGCGTAGATGTTTATTATCCGAATAA |
| | TTCCTTTTTGTCGATAATCAAGCATCAATATTAGATTAGTAGCC |
| | GACCCTCTTCTCTTACCTCTCTAAACAATTTTTCAATATTTCCAT |
| | TTTGGTTGGTAAAAATATTAGAGAACATTTTCTCTATAGAAAT |
| | ATCATGTGTTTATTCAGAATTGTTATTGACTCCATATATTTTTA |
| | ATAATCCAAAACCTATAAATTTAATAGTTTACATTCACAATTAT |
| | TGCTTGTTGCTGTTCTTTTACCTTGTGTTTCTAATTTTTACCGTT |
| | ATTAAATATGCATGTGATTAGTTGAAATATTAAATCTCTAGAA |
| | ATTGAAGGCTGGCTCGAGGTTAGAGATTAAATGGAAAAATGA |
| | AAGTCAACGAAGCATGGTAGCTGTCAAATACGCTCCAACTCAA |
| | AGCATTACTGTCATGATGGAGTTCCTATTGATCCATCGTGAAA |
| | AATTGAACGATATGTTGGCACATGTCGGAATACTTAAAATATC |
| | GAATTAATATAATTGAATTTAACTTTGAAAATTAATCAAATTA |
| | ACTTTCGAAAAGCGCTACCTGACAATTAAAAATACAAGGAGTG |
| | AGCAGTGAACTTAAACTAATCTATTTCAGATCTCTAGGCTTTGA |
| | TAAACTAATTGACAGTTCAACATTTGCAGATCTAACTTCAACG |
| | TTTGCTTCTTGTAGATCTGCAACAAGGCAAACTACCATTTGAG |
| | AGTAAAGAAAAAGGTATAATATAATGTTTTTCTAGTTTTCATTT |
| | TACATGTATTTTTGTGATTCACTTGATTTGAATGAAACATACAT |
| | TACAAGAACTAGAAAACGAATTTGTATTTTTGACTTAGATAAT |
| | TATAAAATGTTAGATGATTTTGTCATAATTTGTAGTATTCACTC |
| | TTGTTTTTAGTAAATATACCTCTTTCTTAAATGTGATTGATTTAC |
| | AGATTATATATTTTAGAATTTTTGCTTATTAAAAGAAAAATTAA |
| | TTAGTGGCGGAAATTTGAGGTGAATTTGTGAAGAATGGAAGAA |
| | GTTGGAAATTC |
| SEQ ID NO: | ATGGATCCGTTTACGGCGGCTGCATTAACCACTGCGGTGACAG |
| 19: | CCACGGTGAGCCTTCTGGTGGAGAACTTGTCGCATCTTATAAG |
| CDS NRC6- | TTATAATTGGAAGTTGTATACAGGATTGAAGAAATCATGCGAA |
| 2a | GATTTGTATGATGAAGTGAAGCGATTAAATGCATTTTTAGTCG |
| | ATAACGCGAATCAGAGAAGTAATAGTACGCAATGGGATGTATT |
| | AGTCGATAAAATTCGACGTACAGTATATAAAGCAGAGGATGTT |
| | GTTGATAAATTATTGATTCAGGGCAAGTTAGACCAAGAGAGTA |
| | ATATAGCTAAAAAGATGTTTCACAAAACTTACAAAAACAGGA |
| | ATTTTACTGAGGAAATCAATGAGATACTTGTAGAGGTGAGGAA |
| | AATCCTTGAGGAAAATCAACATCTGTTTGAGGCAAACCCGACG |
| | ATTGATCATCATCAGCCTGAAAAAGTTGTCCAGGAGGAACAGG |
| | GTTCGTCATTGGAAAATCACGAAGTGGTTGGATTTGATGAAGA |
| | AGCAACGAAAGTGATCAATCGTCTGGTTGAAGGAGCAGAGTG |
| | TCTAGATGTTATCCCGGTTGTAGGAATGCCGGGACTTGGTAAA |
| | ACCACACTGGCAAGAAAAATCTTTAATGATCCTAAGATTTCGC |
| | GAGAATTTTTCAGCTACATTTGGGTTTTCATCGGACAATCAACG |
| | TGTGTAAAAAGGGATATCCTTTTTAATATTCTGAAAGGGTTCA |
| | CAAATTCATTTGATGAATTCAAAAACAGAAATGAGGCAAGCAT |
| | AACTGATGAAATACGTAAGCGTGTGGCTAATGGAGGTAAATGT |
| | CTCATTGTCTTGGATGATGTGTGGGATACAAATGTTGTAGATAT |
| | TGTCAAGACAGTTTTCCCTGATAACAAAAAAGCCCACAGGATC |
| | ATGATGACCACTCGACACGAAGACATTGCTAGATCTGTCAATA |
| | AATATCCCCACAATCTGAAATTTCTGGATGGAGACGAAAGTTT |
| | CCAGCTGCTAGAAAAGAGAGCTTTTGGCGTTAGCCGTTGTCCT |
| | GTTGAGTTAGTAGAACATGGAGAAGCCATTGTAGCAAAATGTA |
| | GTGGAGTACCACTTACAATTGTGGTAATTGCAGGAGCTTTAAG |
| | AGGTCGTACGAGTGAAATTGATTGGAAAGTAGTTAGGGAAAA |
| | TGTGGGGAAGCATCTTATACAAGAAGACAAACTTCAGAGATGT |
| | GTGAATGTTGTGAGATTGAGTTACAATCATTTGCCTCAAGAAA |
| | AAAAATCTTGCTTCTTGTATTTTGGTGCCTTTCCTCAAGGATTT |
| | GATATCCCCGCTTGGAAATTGATTCGACTCTGGATTGCTGAGG |
| | GACTCATAATGTCCAAGTTGTCGGGCAACGAAATTGAAGAGAT |
| | AGCAGAGTATTATCTTAATGACTTTGCCAACAGGAACTTAGTG |
| | ATGGTGATGAAAAAGAAATCTAATGATCGAATAAAAACATGT |
| | CGTGTTCACGACATGTTACATGAGTTTTGCGTTGAAGAGGCTA |
| | CTAGATTGACTCTTTTCAAACAAGTATGTCTCACATCTGATCAA |
| | GACATACAGAACTCAATTACTTGTCGTCGTGTCTCTATTCAATC |
| | ATCTGTTCCTCAAAACTTCATCTCAAAAAAGACAGTTGAAGAA |
| | CATGTTAGGTCATTGTTATGTTTTTCCTCAAAACAAAAACAAGT |
| | TGACTTTTCTAATATCGACGTCAAGCTCATCCCTACCGCATTTC |
| | CACTTATGAGAGTCTTAGACATTGAATCCGTCAAGTTTAGTATT |
| | CCCAGGGAATTTTACCAGCTATTGCACTTGAGGTATATCGCTAT |
| | GTCAGGTGATTTCGAGCAACTTCCTAAACTCTTTACTTCTTTCT |
| | GTAATGCACAAACTCTCATTCTAAATACTTCCAAGCCCACCCTT |
| | GATATAAAAGCTGACATATGGAACATGCCACGTTTGCGTCATC |
| | TGCACACCAACAAACCTGCAATCTTACCACCCCCTACAAGTAG |
| | TAGTAGTAGTAGTACAAATTCTTGTTTGTTGCAAACTCTATCTC |
| | TGGTTACACCAGAAAGCTGCAAGGGAAACGTTCTTTCAAAGGC |
| | TCGTAATGTCAAGAAAATGAGTGTTAAAGGTAATTTGACACCT |
| | TTTCTTGAAACTAGCAAGGGTGAATTTTTCAGCAATTTTCAAGT |
| | GCTAAAGCTCCTGGAAAGTTTAACACTGCTAAATGATGATAAG |
| | AGTAATAAATCTCTTCACCTTCCGTCAGCATTCTCCGAATGTTT |
| | ACCAAATTTGAAGAAGTTAACTCTATCAAAAACAAGGTTTGAC |
| | TGGAATCAGGCATATAGATTAGGGCAGGTGAAAAATCTCCAG |
| | GTCCTAAAACTGAAAGAAAATGCATTCACGGGGCCGTCCTGGA |
| | GGATGGAGCCAGGAGGTTTCAAGAAACTTCAGGTCTTGTGGAT |
| | TGAAATGGCAGATTTCGTGTCGTGGGAGGCATCAAACTGTCCT |
| | TTCCCAAGACTTAGGAGCCTTTTCCTGATCTCCTGTCTTAATCT |
| | TGAGGCTGTGCCACTCGATCTTTCCCATTTGGATAACCTTCAAG |
| | AGATGACGTTGGAAAACACAAGCAAAGCAAGCAAATCTGCAA |
| | GAGAAATAGAATGCGAGAAAAAGAAGAAGCAAGCTGATGATC |
| | CAGAAACTGGCAAATTCAAGCTCACTATTCCCTACTGA |
| SEQ ID NO: | MDPFTAAALTTAVTATVSLLVENLSHLISYNWKLYTGLKKSCEDL |
| 20: | YDEVKRLNAFLVDNANQRSNSTQWDVLVDKIRRTVYKAEDVVD |
| Protein | KLLIQGKLDQESNIAKKMFHKTYKNRNFTEEINEILVEVRKILEEN |
| NRC6-2a | QHLFEANPTIDHHQPEKVVQEEQGSSLENHEVVGFDEEATKVINR |
| | LVEGAECLDVIPVVGMPGLGKTTLARKIFNDPKISREFFSYIWVFI |
| | GQSTCVKRDILFNILKGFTNSFDEFKNRNEASITDEIRKRVANGGK |
| | CLIVLDDVWDTNVVDIVKTVFPDNKKAHRIMMTTRHEDIARSVN |
| | KYPHNLKFLDGDESFQLLEKRAFGVSRCPVELVEHGEAIVAKCSG |
| | VPLTIVVIAGALRGRTSEIDWKVVRENVGKHLIQEDKLQRCVNVV |
| | RLSYNHLPQEKKSCFLYFGAFPQGFDIPAWKLIRLWIAEGLIMSKL |
| | SGNEIEEIAEYYLNDFANRNLVMVMKKKSNDRIKTCRVHDMLHE |
| | FCVEEATRLTLFKQVCLTSDQDIQNSITCRRVSIQSSVPQNFISKKT |
| | VEEHVRSLLCFSSKQKQVDFSNIDVKLIPTAFPLMRVLDIESVKFSI |
| | PREFYQLLHLRYIAMSGDFEQLPKLFTSFCNAQTLILNTSKPTLDIK |
| | ADIWNMPRLRHLHTNKPAILPPPTSSSSSSTNSCLLQTLSLVTPESC |
| | KGNVLSKARNVKKMSVKGNLTPFLETSKGEFFSNFQVLKLLESLT |
| | LLNDDKSNKSLHLPSAFSECLPNLKKLTLSKTRFDWNQAYRLGQ |
| | VKNLQVLKLKENAFTGPSWRMEPGGFKKLQVLWIEMADFVSWE |
| | ASNCPFPRLRSLFLISCLNLEAVPLDLSHLDNLQEMTLENTSKASK |
| | SAREIECEKKKKQADDPETGKFKLTIPY |
| SEQ ID NO: | AGTTTGTCAAATCATCAAAACTACAAATAACAGTTTTGATTTTG |
| 21: Genomic | GACTCTCTTTTTTTTAAAAAAATTTTAATATTATATTATATTTTT |
| NRC6-2c | AAATTAATTTTTTATTGGCCCACGGGCCGGCCCTACCAATATTT |
| | CTCAAGCCCCCCAAATGAGCAGGCTTATTCAGGCTGGACAAAA |
| | AAATCATTTTCTTAAATGGGTTCCAAAAATCTTAGCCCAATCCT |
| | ATTAAATCTCGGGTTAGGCCAGGCCGACCCAACGGACCTAGCC |
| | CATATTGACGGCTCTAGTTAGAACCATGAAAATTATGTGATAT |
| | ATTTTGGTCAAATGTTTCTCACAATGAAACTTAAAAACCTAAT |
| | GAAGGCTAGCTTGAATATATGGCCATACAACAAAATAAAATG |
| | ACAAAACTGATCCCTTATCTTTGCTAGTAGTTTCAATATAGTCC |
| | TTTTAAGTATCAATCAAATACTTTTGATCATTTTTAGTAAGATA |
| | AATTTTGTAGATAAATAACAAATTCGTTGCTAATTCTGTTTAGT |
| | GATAAATTATCAAAGATATTTTTTAACCACAAGCAATTTAACG |
| | ATGAAATTCGTACCTAATTCTATTTTATTTTTATATAAAATATT |
| | ATCGAAACTTTAATGTTTTAAATTCAATTTAAACTGTGAAAATA |
| | TTTTTAAAAAATCTATTTATTTTATTCTTCTATATTTTGTGCTCA |
| | ATATAGTTGGATGTAGTTCACACTTCACAATTGTATTACATAAT |
| | TTTCTTATATCCTTAATCTTTCTTGCATTGTTTTGGGAAAAAAA |
| | TGCAATTTTATAATAATAATTATCGTCATAATAATAATAATTAG |
| | TCAAGTGAATAATAAAAATAATTTCTGTTAAGAATTATTTTAAT |
| | CAAATTATCCACCTTCTTAAGAATCTCATCATTTCAAGTAAACG |
| | TGGATCTGCCAATAAGATAAAGGACCTAGGAAGTGATGATAA |
| | AAAATATTTATTTCAAATTGGGATGATTTCAATAAACGAATAA |
| | AATTCTTGTAACTTAGGAATTAATTTTTTAACTTTTTCTTTTATA |
| | ACAACTCTATTATAAGACTGAATAAAATAATGACTACATTACC |
| | CCCCACTATATTAATTAATTCTATTTAATATAAACTTTACAGAA |
| | AAACCTCGGCAAAGCGAACACCTTGTTTCCCGTGCTCACCAAC |
| | CACAACAATTACGCGGTTAATATCTCAAATGGTTATACAATTTT |
| | AAAAAATTATTCGGTAAAATCATTAAATTTTATTTTATATTATT |
| | AGAGTTACTCAATTTAAGTATATTTAAATCGGTGAAATCACTA |
| | AACTAAATTTATCGATAAATTAAGTGTTGTTAAAATTTTGTTCT |
| | AAACGACTATAGGAAATAGGTTTCTACAAATAACTGTCGGCGC |
| | ATAGTTTCTTTCAAGGAAAAATGCAGAGTGCCAAGTAAGGCCG |
| | AAAAGGGTAAAAAATTATCTATAAAATAAGTCCGGATGGGGG |
| | AATAATAAGACTTGTATAAGTGTGTCTCTGAAATTTCGGACAT |
| | AAATTAGGAGATACTTATGTATTTTCCTTTGTTTCAGTTATTTG |
| | TTTCACCTCTTCCATAATGTTAAATCCTTAATTATATATGTCTA |
| | CCTCTGTACGTCATATTATAGATAGAAAAAAAAATTAAATATG |
| | TGTCTTTATAATCAACACTCTATATAAGTATCACTGAAAGATCT |
| | AGCGACGTTAAATACAACTAAATATTTTTTATTTTGCTACCTTG |
| | AATAATAATTCTCAATTGTTTACAAGCTTTCCAACAACACCCAT |
| | TTATTTTCAACCAATGTGAACCTCTTTCCAACAACACTTAATTT |
| | TTCTATCAATATGAACTTAATTATTCAAATGTTAGCTGTCATTG |
| | GATTATTAAATTGTTAATGTTGTAAGGTGATGGTGTATTATATT |
| | AAGAATTAACTAATAATACTTAAAGACTTGGTTAATCTAATTC |
| | AGAGTCAACATAAAGTTAATTCATTCCACATCTTTATATACAC |
| | ACAACTATGGCAATAACAACATAGCAATTGTCATCTCTAAATT |
| | ACTACTACTATTCTTCTTCTTCATCAATTTAATCCTCAGAAATG |
| | GATCCGTTTACGGCCGCTGCCTTAACCACTGCAGTGACAGCCA |
| | CGGTGAGTCTTCTGGTGGAGAACTTGTCGCATCTTATAAGTTAT |
| | AATTGGAAGTTGTATACAGGATTGAAGAAATCATGCGAAGATT |
| | TGTATGATGAAGTGAAGCGATTAAATGCATTTTTAGCCGATAA |
| | CGCGAATCAGAGAAGTAATAGTACGCAATGGGATGTATTAGTC |
| | GATAAAATTCGACGTACAGTATATAAAGCAGAGGATGTTGTTG |
| | ATAAATTATTGATTCAGGGCAAGTTAGACCAAGAGAGTAATAT |
| | AGCTAGAAAAATGTTTCACAAAACTTACAAAAACAGGAATTTT |
| | ACTGAGGAAATCAATGAGATACTTGTAGAGGTGAGGAAAATC |
| | CTTGATGAAAATCAACATCTGTTTGAGGCAAACCCGACGATTG |
| | ATCATCATCAGCCTGAAAAAGTTGTCCAGGAGGAACAGGTACG |
| | TTTACACGAAATTTCGATTTTAATGTGATTTATTGAAATATGAA |
| | ATGAACTATGATTTGCTCGTTTAGTAAAACTGTATAAGAGTTG |
| | GAGAAATAGTACTTCCTCTGTTTTAATATGCATTATTTGTTTGA |
| | ACTTAGTTAAAGTTTAATCTAAATTATTCAGATCTAGGACAAG |
| | GCATAAGTAAGGCAAAGGATAGAAGTACCTTTCTAGATTATGA |
| | CTCAAATTCCGTAGAATTTTACTTTAGTTAAGGTGTGTCTCTGA |
| | AATTCAGACAATTAAAGGTCACCACCAACCATTATTACATGCA |
| | CAAATCACTATCGATAATTGCCACCATTGGTCAACATTATATAT |
| | CGCTGACCTCCATCTATCATTAACTGTCAGTATGATTCCACTAT |
| | TAATTATCAAATGGTCACCAACAACAACCACAATTAATCACTA |
| | AGGCCAATCACTATACTGTTCACAACCACTATCATCTGTCAAC |
| | ACTATCTAAAATCGATACACACAATCACTATCATTAGTCATTA |
| | CCACCACCGATTGTCACATTTTCTTGTAGTGAATGATGGGGATT |
| | GAAAATATGAAGAATAAAATAGACATGCATAGGATAAGGGGA |
| | CATATTTTTCATAGTTGAATGGGACATTGAAAATTAATAACTTT |
| | CACATGATCATAACTCATTTGGTACCTGATGGAATAATTTCTTG |
| | TTATGATGATACAATCAAGACAAGTAGCCTTAATAAATTGTTT |
| | AATTTGTCCATCTTACAGGGTTCGTCATTGGAAAATCACGAAG |
| | TGGTTGGATTTGATGAAGAAGCAACGAAAGTGATCAATCGTCT |
| | GGTTGAAGGAGCAGAGTGTCTAGATGTTATCCCGGTTGTAGGA |
| | ATGCCGGGACTTGGTAAAACCACACTGGCAAGAAAAATCTTTA |
| | ATGATCCTAAGATTTCGCGAGAATTTTTCAGCTACATTTGGGTT |
| | TTCATCGGACAATCAACGTGTGTAAAAAGGGATATCCTTTTTA |
| | ATATTCTGAAAGGGTTCACAAATTCATTTGATGAATTCAAAAA |
| | CAGAAATGAGGCAAGCATAACTGATGAAATACGTAAGCGTGT |
| | GGCTAATGGAGGTAAATGTCTCATTGTCTTGGATGATGTGTGG |
| | GATACAAATGTTGTAGATATTGTCAAGACAGTTTTCCCTGATA |
| | ACAAAAAAGCCCACAGGATCATGATGACCACTCGACACGAAG |
| | ACATTGCTAGATCTGTCAATAAATATCCCCACAATCTGAAATTT |
| | CTGGATGGAGATGAAAGTTTCCAGCTGCTAGAAAAGAGAGCTT |
| | TTGGCGTTAGCCGTTGTCCTGTTGAGTTAGTAGAACATGGAGA |
| | AGCCATTGTAGCAAAATGTAGTGGAGTACCACTTACAATTGTG |
| | GTAATTGCAGGAGCTTTAAGAGGTCGTACGAGTGAAATTGATT |
| | GGAAAGTAGTTAGGGAAAATGTGGGGAAGCATCTTATACAAG |
| | AAGACAAACTTCAGAGATGTGTGAATGTTGTGAGATTGAGTTA |
| | CAATCATTTGCCTCAAGAAAAAAAATCTTGCTTCTTGTATTTTG |
| | GTGCCTTTCCTCAAGGATTTGATATCCCCGCTTGGAAATTGATT |
| | CGACTCTGGATTGCTGAGGGACTCATAATGTCCAAGTTGTCGG |
| | GCAACGAAATTGAAGAGATAGCAGAGTATTATCTTAATGACTT |
| | TGCCAACAGGAACTTAGTGATGGTGATGAAAAAGAAATCTAAT |
| | GATCGAATAAAAACATGTCGTGTTCACGACATGTTACATGAGT |
| | TTTGCGTTGAAGAGGCTACTAGATTGACTCTTTTCAAACAAGT |
| | ATGTCTCACATCTGATCAAGACATACAGAACTCAATTACTTGT |
| | CGTCGTGTCTCTATTCAATCATCTGTTCCTCAAAACTTCATCTC |
| | AAAAAAGACAGTTGAAGAACATGTTAGGTCATTGTTATGTTTT |
| | TCCTCAAAACAAAAACAAGTTGACTTTTCTAATATCGACGTCA |
| | AGCTCATCCCTACCGCATTTCCACTTATGAGAGTCTTAGACATT |
| | GAATCCGTCAAGTTTAGTATTCCCAGGGAATTTTACCAGCTATT |
| | GCACTTGAGGTATATCGCTATGTCAGGTGATTTCGAGCAACTT |
| | CCTAAACTCTTTACTTCTTTCTGTAATGCACAAACTCTCATTCT |
| | AAATACTTCCAAGCCCACCCTTGATATAAAAGCTGACATATGG |
| | AACATGCCACGTTTGCGTCATCTGCACACCAACAAACCTGCAA |
| | TCTTACCACCCCCTACAAGTAGTAGTAGTAGTAGTACAAATTC |
| | TTGTTTGTTGCAAACTCTATCTCTGGTTACACCAGAAAGCTGCA |
| | AGGGAAACGTTCTTTCAAAGGCTCGTAATGTCAAGAAAATGAG |
| | TGTTAAAGGTAATTTGACACCTTTTCTTGAAACTAGCAAGGGT |
| | GAATTTTTCAGCAATTTTCAAGTGCTAAAGCTCCTGGAAAGTTT |
| | AACACTGCTAAATGATGATAAGAGTAATAAATCTCTTCACCTT |
| | CCGTCAGCATTCTCCGAATGTTTACCAAATTTGAAGAAGTTAA |
| | CTCTATCAAAAACAAGGTTTGACTGGAATCAGGCATATAGATT |
| | AGGGCAGGTGAAAAATCTCCAGGTCCTAAAACTGAAAGAAAA |
| | TGCATTCACGGGGCCGTCCTGGAGGATGGAGCCAGGAGGTTTC |
| | AAGAAACTTCAGGTCTTGTGGATTGAAATGGCAGATTTCGTGT |
| | CGTGGGAGGCATCAAACTGTCCTTTCCCAAGACTTAGGAGCCT |
| | TTTCCTGATCTCCTGTCTTAATCTTGAGGCTGTGCCACTCGATC |
| | TTTCCCATTTGGATAACCTTCAAGAGATGACGTTGGAAAACAC |
| | AAGCAAAGCAAGCAAATCTGCAAGAGAAATAGAATGCGAGAA |
| | AAAGAAGAAGCAAGCTGATCATCCAGAAACTGGCAAATTCAA |
| | GCTCACTATTCCCTACTGAAGCTGATTTCAATGCCACAAAGTG |
| | AAGTTGTTATAAGGTTAGTCCAATTCTCTCCCTCTCGTATCAGT |
| | AAATATCGAATAAGATATTTTTCAATAAGATTGCATCTATCCCT |
| | ATAGTCTCTCCACGCAGAATTAGTAGGTAGCAGTGTGTTGTCTT |
| | GCTGTCTCGTTCATATCAATAGTCATAGTATTGCTCCTATAGTT |
| | TCTTATCCTTCGATTTTTCGTTACTATACGTTGTCCCTTATACCT |
| | CGATATTAGTAATGCTTGTTGTAGTTTCTCTTATGTTACTATCT |
| | GCTGTTTTTGTTATTACCTGTTGTTTCTTGTATTTCCGTCATTTC |
| | TTTTTGGGACTCCTTTGAACTTTCTCCTTGAGACAAAGGTCAAT |
| | CGGAAACAACCTACCTCTCTTTCTTTGAGATAAGGTTCCTCCCC |
| | AGACTCTATTTTGTGGGACTACACTGAGTATATTGTTTTGGGTT |
| | TCGCACTTCACTGAAACATTAAATTGGGCAACATTACTCTGTTA |
| | TACTAATAATACAGATTTTAGGTGCCTTGATATCAAACATAGA |
| | GTAAATTCAATTATTTTCGTGTTCATCACATTCCTCATCTATTTT |
| | TCTCTTACTTATTTTCCTCCTTTTTAGCAGGAGAGTATCGAGGC |
| | ATGGTGATGCATTTGCAGTCTCAGTTTTGATTACCTGTTCCAAT |
| | AAAGAAACTTGTTACAAGTATGTTTTGTTGATTCAAACATGCC |
| | AAGTTTGAAAACGAAGTTTTATATTAATGTTTTTGCGTTTTCTT |
| | TCAGTGCTGAATAATTTGTCTAATCTGTAGTTGATGTTTGGATA |
| | TCTATGTATTGTTCGATTGACACGAGTTGTTTATACTATCATTT |
| | GTTGTTTATGAATATGTAAAACATATTTGATTTATGTTTGCAAC |
| | GTCAACGTGAACATTCAATTAGTCTAATCAAAGTCTAGATCTA |
| | AAATTGATTAATTTAAGTTTATAATTTATTTTGAATTATTATCT |
| | AAAACCGAAGACTTTCTACTCGAAAATCGAAATCATAGCTGAC |
| | AGTCTCCATAAATATATTTGCAATTGAATAACATAATAAGCAT |
| | TTATCAGTTAATTTTATGGAGTTTTTGGATTTACGTGATATTCA |
| | AACATCTCCCGCGCATATTAATTATGTGAAGTAATGAAGTGTG |
| | TAACATAAGTCAAAAGGTGCATAAATAAAGAGTCAAGGGTAA |
| | TAAGACTTTAGTTTACTTTAGGTGTGTCTCTGAAATTTCGATTA |
| | TAATCTACGAGATATTGTGTCTTGTCCCAACATTTAAATAAAAT |
| | GAACAAATAATTTTAATTACCAAATGGATCCATAGTCTGTTTA |
| | GTTGACTAATGGAAAGATTTATAGGAAGGTGAGATTGTTTTAT |
| | CCCTGATTAGAGGTTTTGGTTCGAGTTTTGTGTATACGAAAAA |
| | ATTGTGTTGAAAGCGTCACTTATGAATAGGCCCTATAATGTAT |
| | GATTCGAATTTAGTCTGAGTTCTAATATGAATTTTGAACACATT |
| | GAAAAATTATATCCTTTGTTAAATTTCATGGTTTTAACATCTTG |
| | GAAGTAAAGAAAAAGGTAAAATATTTTTTTTTTCTATTTCATTT |
| | TTTTACATGTAATATTTGAATTCAAAGGCAGAGAGAGTAATAC |
| | CCCCCTTTTATTTTTATTTGTGGATTACATATATACATAAGAAC |
| | TAGAAAAATGTTTATATTTTCGACTTGACAATGATAGAATGTA |
| | AATGTTGGATATATAGAATAATTTGTTGGAGCTTCTACCATTTT |
| | TTGCTTACCGTCTTAAGGTTGAAGGCGTAGATGTTTATTATCCG |
| | AATAATTCCTTTTTGTCGATAATCAAGCATCAATATTAGATTAG |
| | TAGCCGACCCTCTTCTCTTACCTCTCTAAACAATTTTTCAATAT |
| | TTCCATTTTGGTTGGTAAAAATATTAGAGAACATTTTCTCTATA |
| | GAAATATCATGTGTTTATTCAGAATTGTTATTTACTACATATAT |
| | TTTTAATAATTAAAAACCTATAAATTTAATAGTTTACATTCACA |
| | ATTATTGCTTGTTGCTGTTCTTTTGCCTTGTGTTTCTAATTTTTA |
| | CCGTTATTAAATATGCATGTGATTAGTTGAAATATCAAATCTCA |
| | TATTAAATGGAAAAATGAAAGTCAACGAAGCATGGTAGCTGTC |
| | AAATACGCTCCAACTCAAAGCATTACTGTCATGATGGATTTCC |
| | TATTGATCCATCGTGAAAAATTGAACGATATGTTGGCACATGT |
| | CAGAATGCTTAAAATATCGAATTAATATAATTGAATTTAACTTT |
| | GAAAATTAATCAAATTAACTTTCGAAAAGCGCTACCTGACAAT |
| | TAAAAATAGAAGGAGTGAGCAGTGAACTTAAACTAATCTATTT |
| | CAGATCTCTAGAGTTTGATAAACTAATTGACAGTTCAACATTT |
| | GCAGATCTAACTTCAACATTTGCTTCTTGTAAATCTGCAACAAG |
| | GCAAACTACCATTTGAGAGTAAAGAAAAAGGTATAATATAAT |
| | GTTTTTCTAGTTTTCATTTTACATGTATTTTTGTGATTCACTTGA |
| | TTTGAATGAAACATACATTACAAGAACTAGAAAACGAATTTGT |
| | ATTTTTGACTTAGATAATTATAAAATGTTAGATGATTGTCATAA |
| | TTTGTAGTATTCACTCTTGTTTTTAGTAAATATACTCTTTCTTAA |
| | ATGTGATTGATTTACAGATTATATATTTTAGAATTTTTGCTTAT |
| | TAAAAGAAAAATTAATTAGTGGCGGAAATTTGAGGTGAATTCG |
| | TGAAGAATGGAAGAAGTTGGAAATTTGAGAGTTTTACATGTGT |
| | TTTTTAAGGTAGAAGAAACAAAAAGAATCCATTTGATCTAAAA |
| | TTCCAATTGAAAAGTATTAAAAGTTGTTTGGGATCT |
| SEQ ID NO: | ATGGATCCGTTTACGGCCGCTGCCTTAACCACTGCAGTGACAG |
| 22: | CCACGGTGAGTCTTCTGGTGGAGAACTTGTCGCATCTTATAAG |
| CDS NRC6- | TTATAATTGGAAGTTGTATACAGGATTGAAGAAATCATGCGAA |
| 2c | GATTTGTATGATGAAGTGAAGCGATTAAATGCATTTTTAGCCG |
| | ATAACGCGAATCAGAGAAGTAATAGTACGCAATGGGATGTATT |
| | AGTCGATAAAATTCGACGTACAGTATATAAAGCAGAGGATGTT |
| | GTTGATAAATTATTGATTCAGGGCAAGTTAGACCAAGAGAGTA |
| | ATATAGCTAGAAAAATGTTTCACAAAACTTACAAAAACAGGA |
| | ATTTTACTGAGGAAATCAATGAGATACTTGTAGAGGTGAGGAA |
| | AATCCTTGATGAAAATCAACATCTGTTTGAGGCAAACCCGACG |
| | ATTGATCATCATCAGCCTGAAAAAGTTGTCCAGGAGGAACAGG |
| | GTTCGTCATTGGAAAATCACGAAGTGGTTGGATTTGATGAAGA |
| | AGCAACGAAAGTGATCAATCGTCTGGTTGAAGGAGCAGAGTG |
| | TCTAGATGTTATCCCGGTTGTAGGAATGCCGGGACTTGGTAAA |
| | ACCACACTGGCAAGAAAAATCTTTAATGATCCTAAGATTTCGC |
| | GAGAATTTTTCAGCTACATTTGGGTTTTCATCGGACAATCAACG |
| | TGTGTAAAAAGGGATATCCTTTTTAATATTCTGAAAGGGTTCA |
| | CAAATTCATTTGATGAATTCAAAAACAGAAATGAGGCAAGCAT |
| | AACTGATGAAATACGTAAGCGTGTGGCTAATGGAGGTAAATGT |
| | CTCATTGTCTTGGATGATGTGTGGGATACAAATGTTGTAGATAT |
| | TGTCAAGACAGTTTTCCCTGATAACAAAAAAGCCCACAGGATC |
| | ATGATGACCACTCGACACGAAGACATTGCTAGATCTGTCAATA |
| | AATATCCCCACAATCTGAAATTTCTGGATGGAGATGAAAGTTT |
| | CCAGCTGCTAGAAAAGAGAGCTTTTGGCGTTAGCCGTTGTCCT |
| | GTTGAGTTAGTAGAACATGGAGAAGCCATTGTAGCAAAATGTA |
| | GTGGAGTACCACTTACAATTGTGGTAATTGCAGGAGCTTTAAG |
| | AGGTCGTACGAGTGAAATTGATTGGAAAGTAGTTAGGGAAAA |
| | TGTGGGGAAGCATCTTATACAAGAAGACAAACTTCAGAGATGT |
| | GTGAATGTTGTGAGATTGAGTTACAATCATTTGCCTCAAGAAA |
| | AAAAATCTTGCTTCTTGTATTTTGGTGCCTTTCCTCAAGGATTT |
| | GATATCCCCGCTTGGAAATTGATTCGACTCTGGATTGCTGAGG |
| | GACTCATAATGTCCAAGTTGTCGGGCAACGAAATTGAAGAGAT |
| | AGCAGAGTATTATCTTAATGACTTTGCCAACAGGAACTTAGTG |
| | ATGGTGATGAAAAAGAAATCTAATGATCGAATAAAAACATGT |
| | CGTGTTCACGACATGTTACATGAGTTTTGCGTTGAAGAGGCTA |
| | CTAGATTGACTCTTTTCAAACAAGTATGTCTCACATCTGATCAA |
| | GACATACAGAACTCAATTACTTGTCGTCGTGTCTCTATTCAATC |
| | ATCTGTTCCTCAAAACTTCATCTCAAAAAAGACAGTTGAAGAA |
| | CATGTTAGGTCATTGTTATGTTTTTCCTCAAAACAAAAACAAGT |
| | TGACTTTTCTAATATCGACGTCAAGCTCATCCCTACCGCATTTC |
| | CACTTATGAGAGTCTTAGACATTGAATCCGTCAAGTTTAGTATT |
| | CCCAGGGAATTTTACCAGCTATTGCACTTGAGGTATATCGCTAT |
| | GTCAGGTGATTTCGAGCAACTTCCTAAACTCTTTACTTCTTTCT |
| | GTAATGCACAAACTCTCATTCTAAATACTTCCAAGCCCACCCTT |
| | GATATAAAAGCTGACATATGGAACATGCCACGTTTGCGTCATC |
| | TGCACACCAACAAACCTGCAATCTTACCACCCCCTACAAGTAG |
| | TAGTAGTAGTAGTACAAATTCTTGTTTGTTGCAAACTCTATCTC |
| | TGGTTACACCAGAAAGCTGCAAGGGAAACGTTCTTTCAAAGGC |
| | TCGTAATGTCAAGAAAATGAGTGTTAAAGGTAATTTGACACCT |
| | TTTCTTGAAACTAGCAAGGGTGAATTTTTCAGCAATTTTCAAGT |
| | GCTAAAGCTCCTGGAAAGTTTAACACTGCTAAATGATGATAAG |
| | AGTAATAAATCTCTTCACCTTCCGTCAGCATTCTCCGAATGTTT |
| | ACCAAATTTGAAGAAGTTAACTCTATCAAAAACAAGGTTTGAC |
| | TGGAATCAGGCATATAGATTAGGGCAGGTGAAAAATCTCCAG |
| | GTCCTAAAACTGAAAGAAAATGCATTCACGGGGCCGTCCTGGA |
| | GGATGGAGCCAGGAGGTTTCAAGAAACTTCAGGTCTTGTGGAT |
| | TGAAATGGCAGATTTCGTGTCGTGGGAGGCATCAAACTGTCCT |
| | TTCCCAAGACTTAGGAGCCTTTTCCTGATCTCCTGTCTTAATCT |
| | TGAGGCTGTGCCACTCGATCTTTCCCATTTGGATAACCTTCAAG |
| | AGATGACGTTGGAAAACACAAGCAAAGCAAGCAAATCTGCAA |
| | GAGAAATAGAATGCGAGAAAAAGAAGAAGCAAGCTGATCATC |
| | CAGAAACTGGCAAATTCAAGCTCACTATTCCCTACTGA |
| SEQ ID NO: | MDPFTAAALTTAVTATVSLLVENLSHLISYNWKLYTGLKKSCEDL |
| 23: Protein | YDEVKRLNAFLADNANQRSNSTQWDVLVDKIRRTVYKAEDVVD |
| NRC6-2c | KLLIQGKLDQESNIARKMFHKTYKNRNFTEEINEILVEVRKILDEN |
| | QHLFEANPTIDHHQPEKVVQEEQGSSLENHEVVGFDEEATKVINR |
| | LVEGAECLDVIPVVGMPGLGKTTLARKIFNDPKISREFFSYIWVFI |
| | GQSTCVKRDILFNILKGFTNSFDEFKNRNEASITDEIRKRVANGGK |
| | CLIVLDDVWDTNVVDIVKTVFPDNKKAHRIMMTTRHEDIARSVN |
| | KYPHNLKFLDGDESFQLLEKRAFGVSRCPVELVEHGEAIVAKCSG |
| | VPLTIVVIAGALRGRTSEIDWKVVRENVGKHLIQEDKLQRCVNVV |
| | RLSYNHLPQEKKSCFLYFGAFPQGFDIPAWKLIRLWIAEGLIMSKL |
| | SGNEIEEIAEYYLNDFANRNLVMVMKKKSNDRIKTCRVHDMLHE |
| | FCVEEATRLTLFKQVCLTSDQDIQNSITCRRVSIQSSVPQNFISKKT |
| | VEEHVRSLLCFSSKQKQVDFSNIDVKLIPTAFPLMRVLDIESVKFSI |
| | PREFYQLLHLRYIAMSGDFEQLPKLFTSFCNAQTLILNTSKPTLDIK |
| | ADIWNMPRLRHLHTNKPAILPPPTSSSSSSTNSCLLQTLSLVTPESC |
| | KGNVLSKARNVKKMSVKGNLTPFLETSKGEFFSNFQVLKLLESLT |
| | LLNDDKSNKSLHLPSAFSECLPNLKKLTLSKTRFDWNQAYRLGQ |
| | VKNLQVLKLKENAFTGPSWRMEPGGFKKLQVLWIEMADFVSWE |
| | ASNCPFPRLRSLFLISCLNLEAVPLDLSHLDNLQEMTLENTSKASK |
| | SAREIECEKKKKQADHPETGKFKLTIPY |
| SEQ ID NO: | ATGGATCCGTTTACGGCGGCTGCATTAACCACTGCGGTGACAG |
| 24: CDS | CCACGGTGAGCCTTCTGGTGGAGAACTTGTCGCATCTTATAAG |
| NRC6-2b | TTATAATTGGAAGTTGTATACAGGATTGAAGAAATCATGCGAA |
| | GATTTGTATGATGAAGTGAAGCGATTAAATGCATTTTTAGTCG |
| | ATAACGCGAATCAGAGAAGTAATAGTACGCAATGGGATGTATT |
| | AGTCGATAAAATTCGACGTACAGTATATAAAGCAGAGGATGTT |
| | GTTGATAAATTATTGATTCAGGGCAAGTTAGACCAAGAGAGTA |
| | ATATAGCTAAAAAGATGTTTCACAAAACTTACAAAAACAGGA |
| | ATTTTACTGAGGAAATCAATGAGATACTTGTAGAGGTGAGGAA |
| | AATCCTTGAGGAAAATCAACATCTGTTTGAGGCAAACCCGACG |
| | ATTGATCATCATCAGCCTGAAAAAGTTGTCCAGGAGGAACAGG |
| | GTTCGTCATTGGAAAATCACGAAGTGGTTGGATTTGATGAAGA |
| | AGCAACGAAAGTGATCAATCGTCTGGTTGAAGGAGCAGAGTG |
| | TCTAGATGTTATCCCGGTTGTAGGAATGCCGGGACTTGGTAAA |
| | ACCACACTGGCAAGAAAAATCTTTAATGATCCTAAGATTTCGC |
| | GAGAATTTTTCAGCTACATTTGGGTTTTCATCGGACAATCAACG |
| | TGTGTAAAAAGGGATATCCTTTTTAATATTCTGAAAGGGTTCA |
| | CAAATTCATTTGATGAATTCAAAAACAGAAATGAGGCAAGCAT |
| | AACTGATGAAATACGTAAGCGTGTGGCTAATGGAGGTAAATGT |
| | CTCATTGTCTTGGATGATGTGTGGGATACAAATGTTGTAGATAT |
| | TGTCAAGACAGTTTTCCCTGATAACAAAAAAGCCCACAGGATC |
| | ATGATGACCACTCGACACGAAGACATTGCTAGATCTGTCAATA |
| | AATATCCCCACAATCTGAAATTTCTGGATGGAGATGAAAGTTT |
| | CCAGCTGCTAGAAAAGAGAGCTTTTGGCGTTAGCCGTTGTCCT |
| | GTTGAGTTAGTAGAACATGGAGAAGCCATTGTAGCAAAATGTA |
| | GTGGAGTACCACTTACAATTGTGGTAATTGCAGGAGCTTTAAG |
| | AGGTCGTACGAGTGAAATTGATTGGAAAGTAGTTAGGGAAAA |
| | TGTGGGGAAGCATCTTATACAAGAAGACAAACTTCAGAGATGT |
| | GTGAATGTTGTGAGATTGAGTTACAATCATTTGCCTCAAGAAA |
| | AAAAATCTTGCTTCTTGTATTTTGGTGCCTTTCCTCAAGGATTT |
| | GATATCCCCGCTTGGAAATTGATTCGACTCTGGATTGCTGAGG |
| | GACTCATAATGTCCAAGTTGTCGGGCAACGAAATTGAAGAGAT |
| | AGCAGAGTATTATCTTAATGACTTTGCCAACAGGAACTTAGTG |
| | ATGGTGATGAAAAAGAAATCTAATGATCGAATAAAAACATGT |
| | CGTGTTCACGACATGTTACATGAGTTTTGCGTTGAAGAGGCTA |
| | CTAGATTGACTCTTTTCAAACAAGTATGTCTCACATCTGATCAA |
| | GACATACAGAACTCAATTACTTGTCGTCGTGTCTCTATTCAATC |
| | ATCTGTTCCTCAAAACTTCATCTCAAAAAAGACAGTTGAAGAA |
| | CATGTTAGGTCATTGTTATGTTTTTCCTCAAAACAAAAACAAGT |
| | TGACTTTTCTAATATCGACGTCAAGCTCATCCCTACCGCATTTC |
| | CACTTATGAGAGTCTTAGACATTGAATCCGTCAAGTTTAGTATT |
| | CCCAGGGAATTTTACCAGCTATTGCACTTGAGGTATATCGCTAT |
| | GTCAGGTGATTTCGAGCAACTTCCTAAACTCTTTACTTCTTTCT |
| | GTAATGCACAAACTCTCATTCTAAATACTTCCAAGCCCACCCTT |
| | GATATAAAAGCTGACATATGGAACATGCCACGTTTGCGTCATC |
| | TGCACACCAACAAACCTGCAATCTTACCACCCCCTACAAGTAG |
| | TAGTAGTAGTAGTAGTACAAATTCTTGTTTGTTGCAAACTCTAT |
| | CTCTGGTTACACCAGAAAGCTGCAAGGGAAACGTTCTTTCAAA |
| | GGCTCGTAATGTCAAAAAAATGAGTGTTAAAGGTAATTTGACG |
| | CCTTTTCTTGAAACTAGCAAGGGTGAATTTTTCAGCAATTTTCA |
| | AGTGCTAAAGCTCCTGGAAAGTTTAACACTGCTAAATGATGAT |
| | AAGAGTAATAAATCTCTTCACCTTCCGTCAGCATTCTCCGAATG |
| | TTTACCAAATTTGAAGAAGTTAACTCTATCAAAAACAAGGTTT |
| | GACTGGAATCAGGCATATAGATTAGGGCAGGTGAAAAATCTCC |
| | AGGTCCTAAAACTGAAAGAAAATGCATTCACGGGGCCGTCCTG |
| | GAGGATGGAGCCAGGAGGTTTCAAGAAACTTCAGGTCTTGTGG |
| | ATTGAAATGGCAGATTTCGTGTCGTGGGAGGCATCAAACTGTC |
| | CTTTCCCAAGACTTAGGAGCCTTTTCCTGATCTCCTGTCTTAAT |
| | CTTGAGGCTGTGCCACTCGATCTTTCCCATTTGGATAACCTTCA |
| | AGAGATGACGTTGGAAAACACAAGCAAAGCAAGCAAATCTGC |
| | AAGAGAAATAGAATGCGAGAAAAAGAAGAAGCAAGCTGATCA |
| | TCCAGAAACTGGCAAATTCAAGCTCACTATTCCCTACTGA |
| SEQ ID NO: | MDPFTAAALTTAVTATVSLLVENLSHLISYNWKLYTGLKKSCEDL |
| 25: | YDEVKRLNAFLVDNANQRSNSTQWDVLVDKIRRTVYKAEDVVD |
| Protein | KLLIQGKLDQESNIAKKMFHKTYKNRNFTEEINEILVEVRKILEEN |
| NRC6-2b | QHLFEANPTIDHHQPEKVVQEEQGSSLENHEVVGFDEEATKVINR |
| | LVEGAECLDVIPVVGMPGLGKTTLARKIFNDPKISREFFSYIWVFI |
| | GQSTCVKRDILFNILKGFTNSFDEFKNRNEASITDEIRKRVANGGK |
| | CLIVLDDVWDTNVVDIVKTVFPDNKKAHRIMMTTRHEDIARSVN |
| | KYPHNLKFLDGDESFQLLEKRAFGVSRCPVELVEHGEAIVAKCSG |
| | VPLTIVVIAGALRGRTSEIDWKVVRENVGKHLIQEDKLQRCVNVV |
| | RLSYNHLPQEKKSCFLYFGAFPQGFDIPAWKLIRLWIAEGLIMSKL |
| | SGNEIEEIAEYYLNDFANRNLVMVMKKKSNDRIKTCRVHDMLHE |
| | FCVEEATRLTLFKQVCLTSDQDIQNSITCRRVSIQSSVPQNFISKKT |
| | VEEHVRSLLCFSSKQKQVDFSNIDVKLIPTAFPLMRVLDIESVKFSI |
| | PREFYQLLHLRYIAMSGDFEQLPKLFTSFCNAQTLILNTSKPTLDIK |
| | ADIWNMPRLRHLHTNKPAILPPPTSSSSSSSTNSCLLQTLSLVTPES |
| | CKGNVLSKARNVKKMSVKGNLTPFLETSKGEFFSNFQVLKLLESL |
| | TLLNDDKSNKSLHLPSAFSECLPNLKKLTLSKTRFDWNQAYRLGQ |
| | VKNLQVLKLKENAFTGPSWRMEPGGFKKLQVLWIEMADFVSWE |
| | ASNCPFPRLRSLFLISCLNLEAVPLDLSHLDNLQEMTLENTSKASK |
| | SAREIECEKKKKQADHPETGKFKLTIPY |
| SEQ ID NO: | ATGGATCCGTTTACGGCCGCTGCCTTAACCACTGCAGTGACAG |
| 26: CDS | CCACGGTGAGTCTTCTGGTGGAGAACTTGTCGCATCTTATAAG |
| NRC6-3 | TTATAATTGGAAGTTGTATACAGGATTGAAGAAATCATGCGAA |
| | GATTTGTATGATGAAGTGAAGCGATTAAATGCATTTTTAGTCG |
| | ATAACGCGAATCAGAGAAGTAATAGTACGCAATGGGATGTATT |
| | AGTCGATAAAATTCGACGTACAGTATATAAAGCAGAGGATGTT |
| | GTTGATAAATTATTGATTCAGGGCAAGTTAGACCAAGAGAGTA |
| | ATATAGCTAAAAAGATGTTTCACAAAACTTACAAAAACAGGA |
| | ATTTTACTGAGGAAATCAATGAGATACTTGTAGAGGTGAGGAA |
| | AATCCTTGAGGAAAATCAACATCTGTTTGAGGCAAACCCGACG |
| | ATTGATCATCATCAGCCTGAAAAAGTTGTCCAGGAGGAACAGG |
| | GTTCATCGTTGGAAAATCACGAAGTGGTAGGATTTGATGAAGA |
| | AGCAACGAAAGTGATCAATCGTCTGGTTGAAGGAGCAGAGCG |
| | TCTAGATGTTATCCCGGTTGTAGGAATGCCGGGACTTGGTAAA |
| | ACCACACTGGCAAGAAAAATCTTTAATGATCCGAAGATTTCGC |
| | GAGAATTTTTCAGCTACATTTGGGTTTTCATCGGACAATCAACG |
| | TGTGTAAAAAGGGATATCCTTTTTAATATTCTGAAAGGGTTCA |
| | CAAATTCATTTGATGAATTCAAAAACAGAAATGAGGCAGACAT |
| | AACTGATGAAATACGTAAGCGTGTGGCTAATGGAGGTAAATGT |
| | CTCATTGTCTTGGATGATGTGTGGGATCCAAATGTTGTAGATAT |
| | TGTCAAGACAGTTTTCCCTGATAACAAAAAAGCCCACAGGATC |
| | ATGATGACCACTCGACACGAAGACATTGCTAGATCTGTCAATA |
| | AATATCCTCACAATCTGAAATTTCTGGATGGAGATGAAAGTTT |
| | CCAGCTGCTAGAAAAGAGAGCTTTTGGCGTTAGCCGTTGTCCA |
| | GTTGAGTTAGTAGAACATGGAGAAGCCATTGTAGCAAAATGTA |
| | GTGGAGTACCACTTACAATTGTGGTAATTGCAGGAGCTTTAAG |
| | AGGTCGTACGAGTGAAATTGATTGGAAAGTAGTTAGGGAAAA |
| | TGTGGGGAAGCATCTTATACAAGAAGACAAACTTCAGAGATGT |
| | GTGAATGTTGTGAGATTGAGTTACAATCATTTGCCTCAAGAAA |
| | AAAAATCTTGCTTCTTGTATTTTGGTGCCTTTCCTCAAGGATTC |
| | GATATCCCCGCTTGGAAGTTGATTCGACTCTGGATTGCTGAGG |
| | GACTCATAATGTCCAAGTTGTCAGGCAACGAAATTGAAGAGAT |
| | AGCAGAGTATTATCTTAATGACTTTGCCAACAGGAACTTAGTG |
| | ATGGTGATGAAAAAGAAATCTAATGATCGAATAAAAACATGT |
| | CGTGTTCACGACATGTTACATGAGTTTTGCGTTGAAGAGGCTA |
| | CTAGATTGACTCTTTTCAAACAAGTATGTCTCACATCTGATCAA |
| | GACATACAAAACTCAATTGCTTGTCGTCGTGTCTCTATTCAATC |
| | ATCTGTTCCTCAAAACTTCATCTCAAAAAAGACAGTTGAAGAG |
| | CATGTTAGGTCATTTTTATGTTTTTCCTCAAAACAAAAACAAGT |
| | TGACTTTTCTAATATCGACGTCAAGCTCATCCCTGCCGCATTTC |
| | CACTTATGAGAGTCTTAGACATTGAATCCGTCAAGTTTAGTATT |
| | CCCAGGGAATTTTACCAGCTATTGCACTTGAGGTATATCGCTAT |
| | CTCAGGTGATTTCGAGCAACTTCCTAAACTCTTTACTTCTTTCT |
| | GTAATGCACAAACTCTCATTCTAAATACTTCCAAGCCCACCCTT |
| | GATATAAAAGCTGACATATGGAACATGCCACGTTTGCGTCATC |
| | TGCGCACCAACAAACCTGCAATCTTACCACCCCCTACAAGTAG |
| | TAGTAGTAGTAGTAGTACAAATTCTTGTTTTTTGCAAACTCTAT |
| | CTCTGGTTACACCAGAAAGCTGCAAGGGAAATGTTCTTTCAAA |
| | AGCTCGTAATGTCAAAAAAATGAGTGTTAAAGGTAATTTGACG |
| | CCTTTTCTTGAAACTAGCAAGGGTGAATTTTTCAGCAATTTTCA |
| | AGTGCTAAAGCTCCTGGAAAGTTTAACACTGCTAAATGATGAT |
| | AAGAGTAACAAATCTCTTCACCTTCCGTCAGCATTCTCCGAAT |
| | GTTTACCAAATTTGAAGAAGTTAACTCTATCAAAAACAAGGTT |
| | TGACTGGAATCAGGCATATAGATTGGGGCAGGTGAAAAATCTC |
| | CAGGTCCTAAAACTGAAAGAAAATGCATTCATGGGGCCGTCCT |
| | GGAGGATGGAGCCAGGAGGTTTCAAGAAACTTCAGGTCTTGTG |
| | GATTGAAATGGCAGATTTCGTGTCGTGGGAGGCATCAAACTGT |
| | CCTTTCCCAAGACTTAGGAGCCTTTTCCTGATCTCCTGTCTTAA |
| | TCTTGAGGCTGTGCCACTCGATCTTGCCCATTTGGATAACCTTC |
| | AAGAGATGACGTTGGAAAACACAAGCAAAGCAAGCAAATCTG |
| | CAAGAGAAATAGAATGCGAGAAAAAGAAGAAGCAAGCTGATC |
| | ATCCAGAAAGTGGCAAATTCAAGCTCACTATTCCCTACTGA |
| SEQ ID NO: | MDPFTAAALTTAVTATVSLLVENLSHLISYNWKLYTGLKKSCEDL |
| 27: | YDEVKRLNAFLVDNANQRSNSTQWDVLVDKIRRTVYKAEDVVD |
| Protein | KLLIQGKLDQESNIAKKMFHKTYKNRNFTEEINEILVEVRKILEEN |
| NRC6-3 | QHLFEANPTIDHHQPEKVVQEEQGSSLENHEVVGFDEEATKVINR |
| | LVEGAERLDVIPVVGMPGLGKTTLARKIFNDPKISREFFSYIWVFI |
| | GQSTCVKRDILFNILKGFTNSFDEFKNRNEADITDEIRKRVANGGK |
| | CLIVLDDVWDPNVVDIVKTVFPDNKKAHRIMMTTRHEDIARSVN |
| | KYPHNLKFLDGDESFQLLEKRAFGVSRCPVELVEHGEAIVAKCSG |
| | VPLTIVVIAGALRGRTSEIDWKVVRENVGKHLIQEDKLQRCVNVV |
| | RLSYNHLPQEKKSCFLYFGAFPQGFDIPAWKLIRLWIAEGLIMSKL |
| | SGNEIEEIAEYYLNDFANRNLVMVMKKKSNDRIKTCRVHDMLHE |
| | FCVEEATRLTLFKQVCLTSDQDIQNSIACRRVSIQSSVPQNFISKKT |
| | VEEHVRSFLCFSSKQKQVDFSNIDVKLIPAAFPLMRVLDIESVKFSI |
| | PREFYQLLHLRYIAISGDFEQLPKLFTSFCNAQTLILNTSKPTLDIK |
| | ADIWNMPRLRHLRTNKPAILPPPTSSSSSSSTNSCFLQTLSLVTPES |
| | CKGNVLSKARNVKKMSVKGNLTPFLETSKGEFFSNFQVLKLLESL |
| | TLLNDDKSNKSLHLPSAFSECLPNLKKLTLSKTRFDWNQAYRLGQ |
| | VKNLQVLKLKENAFMGPSWRMEPGGFKKLQVLWIEMADFVSWE |
| | ASNCPFPRLRSLFLISCLNLEAVPLDLAHLDNLQEMTLENTSKASK |
| | SAREIECEKKKKQADHPESGKFKLTIPY |
| SEQ ID NO: | ATGGATCCGTTTACGGCCGCTGCATTAACCACTGCGGTGACAG |
| 28: CDS | CCACGGTGAGCCTTCTGGTGGAGAACTTGTCGCATCTTATAAG |
| NRC6-1a | TTATAATTGGAAGTTGTATACAGGATTGAAGAAATCATGCGAA |
| | GATTTGTATGATGAAGTGAAGCGATTAAATGCATTTTTAGTCG |
| | ATAACGCGAATCAGAGAAGTAATAGTACGCAATGGGATGTAC |
| | TAGTCGATAAAATTCGACGTACAGTATATAAAGCAGAGGATGT |
| | TGTTGATAAATTATTGATTCAGGGCAAGTTAGACCAAGAGAGT |
| | AATATAGCTAAAAAGATGTTTCACAAAACTTACAAAAACAGG |
| | AATTTTACTGAGGAAATCAATGAGATACTTGTAGAGGTGAGGA |
| | AAATCCTTGAGGAAAATCAACATCTGTTTGAGGCAAACCCGAC |
| | GATTGATCATCATCAGCCTGAAAAAGTTGTCCAGGAGGAACAG |
| | GGTTCGTCATTGGAAAATCACGAAGTGGTTGGATTTGATGAAG |
| | AAGCAACGAAAGTGATCAATCGTCTGGTTGAAGGAGCAGAGT |
| | GTCTAGATGTTATCCCGGTTGTAGGAATGCCGGGACTTGGTAA |
| | AACCACACTGGCAAGAAAAATCTTTAATGATCCTAAGATTTCG |
| | CGAGAATTTTTCAGCTACATTTGGGTTTTCATCGGACAATCAAC |
| | GTGTGTAAAAAGGGATATCCTTTTTAATATTCTGAAAGGGTTC |
| | ACAAATTCATTTGATGAATTCAAAAACAGAAATGAGGCAAGC |
| | ATAACTGATGAAATACGTAAGCGTGTGGCTAATGGAGGTAAAT |
| | GTCTCATTGTCTTGGATGATGTGTGGGATCCAAATGTTGTAGAT |
| | ATTGTCAAGACAGTTTTCCCTGATAACAAAAAAGCCCACAGGA |
| | TCATGATGACCACTCGACACGAAGACATTGCTAGATCTGTCAA |
| | TAAATATCCCCACAATCTGAAATTTCTGGATGGAGATGAAAGT |
| | TTCCAGCTGCTAGAAAAGAGAGCTTTTGGCGTTAGCCGTTGTC |
| | CTGTTGAGTTAGTAGAACATGGAGAAGCCATTGTAGCAAAATG |
| | TAGTGGAGTACCACTTACAATTGTGGTAATTGCAGGAGCTTTA |
| | AGAGGTCGTACGAGTGAAATTGATTGGAAAGTAGTTAGGGAA |
| | AATGTGGGGAAGCATCTTATACAAGAAGACAAACTTCAGAGA |
| | TGTGTGAATGTTGTGAGATTGAGTTACAATCATTTGCCTCAAG |
| | AAAAAAAATCTTGCTTCTTGTATTTTGGTGCCTTTCCTCAAGGA |
| | TTTGATATCCCCGCTTGGAAATTGATTCGACTCTGGATTGCTGA |
| | GGGACTCATAATGTCCAAGTTGTCAGGCAACGAAATTGAAGAG |
| | ATAGCAGAGTATTATCTTAATGACTTTGCCAACAGGAACTTAG |
| | TGATGGTGATGAAAAAGAAATCTAATGATCGAATAAAAACAT |
| | GTCGTGTTCACGACATGTTACATGAGTTTTGCGTTGAAGAGGC |
| | TACTAGATTGACTCTTTTCAAACAAGTATGTCTCACATCTGATC |
| | AAGACATACAGAACTCAATTACTTGTCGTCGTGTCTCTATTCAA |
| | TCATCTGTTCCTCAAAACTTCATCTCAAAAAAGACAGTTGAAG |
| | AACATGTTAGGTCATTGTTATGTTTTTCCTCAAAACAAAAACA |
| | AGTTGACTTTTCTAATATCGACGTCAAGCTCATCCCTACCGCAT |
| | TTCCACTTATGAGAGTCTTAGACATTGAATCCGTCAAGTTTAGT |
| | ATTACCAGGGAATTTTACCAGCTATTGCACTTGAGGTATATCG |
| | CTATCTCAGGTGATTTCGAGCAACTTCCTAAACTCTTTACTTCT |
| | TTCTGTAATGCACAAACTCTCATTCTAAATACTTCCAAGCCCAC |
| | CCTTGATATAAAAGCTGACATATGGAACATGCCACGTTTGCGT |
| | CATCTGCGCACCAACAAACCTGCAATCTTACCACCCCCTACAA |
| | GTAGTAGTAGTAGTAGTAGTACAAATTCTTGTTTTTTGCAAACT |
| | CTATCTCTGGTTACACCAGAAAGCTGCAAGGGAAATGTTCTTT |
| | CAAAAGCTCGTAATGTCAAAAAAATGAGTGTTAAAGGTAATTT |
| | GACGCCTTTTCTTGAAACTAGCAAGGGTGAATTTTTCAGCAATT |
| | TTCAAGTGCTAAAGCTCCTGGAAAGTTTAACACTGCTAAATGA |
| | TGATAAGAGTAACAAATCTCTTCACCTTCCGTCAGCATTCTCCG |
| | AATGTTTACCAAATTTGAAGAAGTTAACTCTATCAAAAACAAG |
| | GTTTGACTGGAATCAGGCATATAGATTGGGGCAGGTGAAAAAT |
| | CTCCAGGTCCTAAAAATGAAAGAAAATGCATTCATGGGGCCGT |
| | CCTGGAGGATGGAGCCAGGAGGTTTCAAGAAACTTCAGGTCTT |
| | GTGGATTGAAATGGCAGATTTCGTGTCGTGGGAGGCATCAAAC |
| | TGTCCTTTCCCAAGACTTAGGAGCCTTTTCCTGATCTCCTGTCT |
| | TAATCTTGAGGCTGTGCCACTCGATCTTGCCCATTTGGATAACC |
| | TTCAAGAGATGACGTTGGAAAACACAAGCAAAGCAAGCAAAT |
| | CTGCAAGAGAAATAGAATGCGAGAAAAAGAAGAAGCAAGCTG |
| | ATCATCCAGAAAGTGGCAAATTCAAGCTCACTATTCCCTACTG |
| | A |
| SEQ ID NO: | MDPFTAAALTTAVTATVSLLVENLSHLISYNWKLYTGLKKSCEDL |
| 29: | YDEVKRLNAFLVDNANQRSNSTQWDVLVDKIRRTVYKAEDVVD |
| Protein | KLLIQGKLDQESNIAKKMFHKTYKNRNFTEEINEILVEVRKILEEN |
| NRC6-1a | QHLFEANPTIDHHQPEKVVQEEQGSSLENHEVVGFDEEATKVINR |
| | LVEGAECLDVIPVVGMPGLGKTTLARKIFNDPKISREFFSYIWVFI |
| | GQSTCVKRDILFNILKGFTNSFDEFKNRNEASITDEIRKRVANGGK |
| | CLIVLDDVWDPNVVDIVKTVFPDNKKAHRIMMTTRHEDIARSVN |
| | KYPHNLKFLDGDESFQLLEKRAFGVSRCPVELVEHGEAIVAKCSG |
| | VPLTIVVIAGALRGRTSEIDWKVVRENVGKHLIQEDKLQRCVNVV |
| | RLSYNHLPQEKKSCFLYFGAFPQGFDIPAWKLIRLWIAEGLIMSKL |
| | SGNEIEEIAEYYLNDFANRNLVMVMKKKSNDRIKTCRVHDMLHE |
| | FCVEEATRLTLFKQVCLTSDQDIQNSITCRRVSIQSSVPQNFISKKT |
| | VEEHVRSLLCFSSKQKQVDFSNIDVKLIPTAFPLMRVLDIESVKFSI |
| | TREFYQLLHLRYIAISGDFEQLPKLFTSFCNAQTLILNTSKPTLDIK |
| | ADIWNMPRLRHLRTNKPAILPPPTSSSSSSSTNSCFLQTLSLVTPES |
| | CKGNVLSKARNVKKMSVKGNLTPFLETSKGEFFSNFQVLKLLESL |
| | TLLNDDKSNKSLHLPSAFSECLPNLKKLTLSKTRFDWNQAYRLGQ |
| | VKNLQVLKMKENAFMGPSWRMEPGGFKKLQVLWIEMADFVSW |
| | EASNCPFPRLRSLFLISCLNLEAVPLDLAHLDNLQEMTLENTSKAS |
| | KSAREIECEKKKKQADHPESGKFKLTIPY |

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: M. enterolobii Disease Testing

Determining resistance of a tomato plant was done by counting egg masses. The tomato plants to be tested were sown in trays in a greenhouse, after 2 weeks the plants were transferred to 660 cc pots filled with a sandy soil mixture. As a susceptible control plant Dometica RZ was introduced in the test and as a positive control plants grown from the deposit NCIMB 43515 were introduced in the test. Next to the roots, holes were made with a 1 ml pipet tip and filled with 1 ml of suspension comprising 500J2 of the M. enterolobii inoculum. After inoculation the plants were grown in a growth chamber (23° C., 14 h. light) and watered three times a week. Six weeks after inoculation the plants were removed from the soil and the roots were washed with tap water in order to remove the soil. The egg masses were counted and the scoring was done according to the symptoms as presented in Table 2. An M. enterolobii resistant tomato plant has a score of 0, 1 or 2, preferably 0 or 1, when scoring according to Table 2 is used.

TABLE 2
scales M. enterolobii resistance scores

| Score | Number of Egg Masses | Type of resistance |
|---|---|---|
| 0 | 0 | Highly resistant |
| 1 | 1-3 | Resistant |
| 2 | 4-20 | Intermediate resistant |
| 3 | 21-100 | Susceptible |
| 4 | >100 | Highly Susceptible |

### Example 2: Identification of the Nucleic Acid Molecule Capable of Conferring M. enterolobii Resistance

Various internally developed Solanum lycopersicum populations that segregated for M. enterolobii resistance were finemapped to a small region on chromosome 4 that contained only 10 potential genes, which were likely to contribute to the M. enterolobii resistance. Whole genome sequences were available in-house for the backgrounds of the resistant and susceptible lines that were used in the development of these populations. Some of the genes had a premature stop codon and therefore a functional protein was not expressed. Only the genes present in the region of interest and expressing a functional protein were considered to be candidate genes. Among the remaining genes in the region of interest was the MeR1 gene of the invention, which had various polymorphisms between susceptible and resistant material. Segregating material showed that the MeR1 gene of the invention segregates together with resistance. Through analysis of the correlation in segregation of phenotypes and genotypes it was determined that the specific the MeR1 gene was the cause of the M. enterolobii resistance of the resistant Solanum lycopersicum plants. During the analysis it was also seen that NRC6 segregates together with the MeR1 gene of the invention for the resistance to the M. enterolobii resistance. NRC6 comprise the MADA motif as disclosed in Adachi et al. ((2019) An N-terminal motif in NLR immune receptors is functionally conserved across distantly related plant species; Elife. 27; 8) and was therefore considered to be NLR helper protein.

The expression of the MeR1 protein encoded by the MeR1 gene of the invention was determined by RNA sequencing of material of the leaves and the roots of infected plants as well as non-infected plants of Solanum lycopersicum plants that are susceptible to M. enterolobii and plants of the invention. This lead to the conclusion that the MeR1 protein is expressed in the roots of the plant and is not present in the leaves of the plant.

### Example 3: Modifying the MeR1 Gene of the Invention

A functional MeR1 protein in a plant is required for having the resistance to nematodes and in particular the resistance to M. enterolobii. Therefore, inhibiting the expression of the MeR1 protein by mutating the MeR1 gene of the invention of a resistant plant will lead to a plant which is susceptible to M. enterolobii.

For the inhibition of the expression of the MeR1 protein a CRISPR/Cas experiment was performed. Two designed guide RNAs were incorporated in a plasmid. The two guide RNAs were designed to target two different locations in the coding sequence of the MeR1 gene. An Agrobacterium tumefaciens-mediated transformation with the CRISPR/Cas construct was performed on young tomato (Solanum lycopersicum) plantlets after growing them in a sterile environment for 6 days. The transformation was performed on plantlets which are resistant and which were obtained by crossing the Solanum pimpinellifolium source with a susceptible Solanum lycopersicum line to obtain a F1 generation. Subsequently, the F1 plants were back-crossed 3 times to obtain a BC3 population and finally selfed two times to obtain a F3BC3 population.

In the resistant plantlets, one mutant plant with a 236 bp deletion in the coding sequence of the MeR1 gene was found and selected. The deletion led to the expression of a non-functional MeR1 protein, thereby inhibiting its expression. The selected plant was selfed in order to obtain a segregating population. Confirmation of the deletion was performed by using a simple PCR experiment. For the PCR experiment, the isolated genomic DNA was used together with the forward primer AGGAATGTATAAATACTCTGACA (SEQ ID NO:31) and reverse primer GCCTAGCAACTAATGTATCT (SEQ ID NO:32). Said primer pair has been designed to amplify the MeR1 amplicon with and without the mutation. The conditions were as follows:

Primer pair with SEQ ID NO:31 and SEQ ID NO:32:
3 minutes at 95° C. (initial denaturing step)
40 amplification cycles, each cycle consisting of 30 seconds denaturation at 95° C., 30 seconds annealing at 60° C., and 75 seconds extension at 75° C.
10 minutes at 72° C. (final extension step).

The PCR products were visualized on agarose gel; plants with the mutation showed an amplicon size of 820 bp and plants without the mutation showed an amplicon of 1100 bp.

By this PCR experiment, 9 plants homozygous for the mutation and 16 plants with a wild-type version of the MeR1 gene were selected for the experiment.

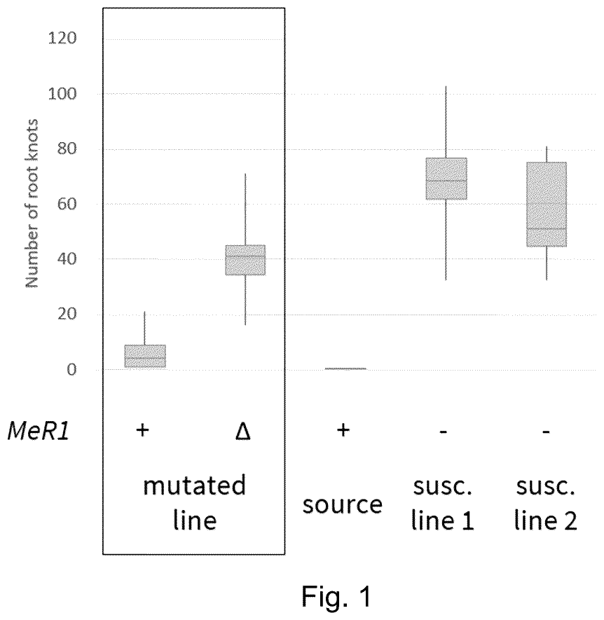

Mutant plants comprising the deletion in the MeR1 gene homozygously as well as plants comprising a functional MeR1 gene in the same genetic background were tested for resistance by counting the number of root knots. Counting the number of root knots is different of counting egg masses as described in Example 1 as it is performed at a different time after inoculation. The tomato plants to be tested were sown in trays in a greenhouse, after 2 weeks the plants were transferred to 900 cc square pots filled with a sandy soil mixture. Next to the roots, holes were made with a 1 ml pipet tip and filled with 1 ml of suspension comprising 500J2 of the M. enterolobii inoculum. After inoculation the plants were grown in a controlled greenhouse (23° C., minimum 14 h. light) and watered three times a week. 28 days after inoculation the plants were removed from the soil and the roots were tapped free of soil as much as possible. Plants with the deletion had a significant increase in susceptibility to M. enterolobii i.e. an increase in numbers of root knots, as compared to a plant with the same background but without the deletion in the MeR1 gene. The results represented in FIG. 1 show that the inhibition of the expression of a functional MeR1 gene of a resistant plant leads to a more susceptible plant. Therefore the MeR1 gene of the invention confers resistance to M. enterolobii to tomato plants. A resistant tomato plant can for example be grown from the deposit NCIMB 43515.

The invention is further described by the following numbered paragraphs:

1. A nucleic acid molecule encoding an MeR1 protein, which confers resistance to root-knot nematode when present in a plant of the Solanaceae family, wherein said nucleic acid molecule has a nucleotide sequence which is:
a) a nucleotide sequence comprising a coding sequence according to SEQ ID NO: 2, 3, 4, 5, 6, 7, 8 or 9, or a coding sequence which has at least 90% sequence identity with the sequence according to SEQ ID NO: 2, 3, 4, 5, 6, 7, 8 or 9; or
b) a nucleotide sequence encoding a protein having an amino acid sequence according to SEQ ID NO: 10, 11, 12, 13, 14, 15, 16 or 17, or a protein having an amino acid sequence which has at least 85% sequence identity with the amino acid sequence according to SEQ ID NO: 10, 11, 12, 13, 14, 15, 16 or 17.

2. The nucleic acid of paragraph 1, wherein the sequence identity with the sequence according to SEQ ID NO: 2, 3, 4, 5, 6, 7, 8 or 9 in order of increased preference is 91%, 92%, 93%, 94%, 95%, 96%, 97%, 98%, 99%.

3. The nucleic acid of paragraph 1 or paragraph 2, wherein the sequence identity with the amino acid sequence according to SEQ ID NO: 10, 11, 12, 13, 14, 15, 16 or 17 in order of increased preference is 85%, 86%, 87%, 88%, 89%, 90%, 91%, 92%, 93%, 94%, 95%, 96%, 97%, 98%, 99%.

4. A nucleic acid molecule encoding a NRC6 protein, which contributes to resistance to root-knot nematode when present in a plant of the Solanaceae family together with the nucleic acid molecule of any one on the paragraphs 1-3, wherein said nucleic acid molecule has a nucleotide sequence which is:
a) a nucleotide sequence comprising a coding sequence according to SEQ ID NO: 19, or a coding sequence having at least 98% sequence identity with the sequence according to SEQ ID NO: 19; or
b) a nucleotide sequence encoding a protein having an amino acid sequence according to SEQ ID NO: 20, or a protein having an amino acid sequence which has at least 98% sequence identity with the amino acid sequence according to SEQ ID NO: 20.

5. The nucleic acid molecule of any one of the paragraphs 1-4, which confers resistance to root-knot nematode when present in a Solanum lycopersicum plant.

6. A plant of the Solanaceae family, in particular a Solanum lycopersicum plant, comprising the nucleic acid molecule of any one of the paragraphs 1-3 or 5, wherein the plant is resistant to a root-knot nematode.

7. A plant of the Solanaceae family, in particular a Solanum lycopersicum plant, comprising the nucleic acid molecule of any one of the paragraphs 1-3 or 5 and at least one nucleic acid molecule of paragraph 4 or 5, wherein the plant is resistant to a root-knot nematode.

8. A plant of the Solanaceae family, in particular a Solanum lycopersicum plant, comprising the nucleic acid molecule of any one of the paragraphs 1-3 or 5 and two nucleic acid molecules of paragraph 4 or 5, wherein the plant is resistant to a root-knot nematode.

9. The plant of any one of the paragraphs 6-8, wherein the plant is resistant to Meloidogyne enterolobii.

10. The plant of paragraph 8 or 9, which is a Solanum lycopersicum plant, wherein the nucleic acid molecules are as comprised in the genome of a Solanum lycopersicum plant representative seed of which was deposited with the NCIMB under deposit number NCIMB 43515.

11. A rootstock of a plant of the Solanaceae family comprising the nucleic acid molecule of any one of the paragraphs 1-3 or 5, and optionally a nucleic acid molecule of paragraph 4 or 5.

12. A seed, wherein a plant of the Solanaceae family, in particular a Solanum lycopersicum plant, grown from the seed comprises the nucleic acid molecule as defined in any one of the paragraphs 1-3 or 5, and optionally a nucleic acid molecule of any one of the paragraphs 4 and 5.

13. The seed of paragraph 11, wherein the Solanum lycopersicum plant grown from the seed is resistant to Meloidogyne enterolobii,

14. Method for selecting a plant of the Solanaceae family carrying the nucleic acid molecule of in any one of the paragraphs 1-3 or 5 and optionally a nucleic acid molecule of paragraph 4 or 5, comprising determining the presence of the nucleic acid molecule as defined in any one of the paragraphs 1-3 or 5 and optionally a nucleic acid molecule of paragraph 4 or 5, or parts thereof, in the plant of the Solanaceae family, and selecting the Solanaceae plant if it comprises the nucleic acid molecule of any one of the paragraphs 1-3 or 5 and optionally the nucleic acid molecule of paragraph 4 or 5.

15. The method of paragraph 14, further comprising the step of determining whether the plant shows resistance against a root-knot nematode.

16. The method of paragraph 14 or 15, wherein the plant is a Solanum lycopersicum plant.

17. The method of any one of the paragraphs 14 to 16, wherein the root-knot nematode is Meloidogyne enterolobii.

18. A method for producing a plant of the Solanaceae family, in particular a Solanum lycopersicum plant, having resistance to root-knot nematode comprising:
(a) crossing a plant comprising the nucleic acid molecule of any one of the paragraphs 1-3 and 5 and optionally a nucleic acid molecule of paragraph 4 or 5, with another plant;
(b) optionally performing one or more rounds of selfing and/or crossing;
(c) selecting after one or more rounds of selfing and/or crossing for a plant that comprises said nucleic acid molecule of any one of the paragraphs 1-3 or 5 and optionally a nucleic acid molecule of paragraph 4 or 5.

19. The method of paragraph 18, wherein the selection of a plant of the Solanaceae family which is resistant to root-knot nematode and comprises the nucleic acid molecule of any one of the paragraphs 1-3 or 5 and optionally a nucleic acid molecule of paragraph 4 or 5, further comprises determining the presence of the nucleic acid molecule according the method of paragraph 14.

20. The method of any one of the paragraphs 18 to 19, wherein the root-knot nematode is Meloidogyne enterolobii.

21. Use of the nucleic acid molecule of any one of the paragraphs 1-3 or 5 and optionally together with a nucleic acid molecule of paragraph 4 or 5 for producing a plant of the Solanaceae family, in particular a Solanum lycopersicum plant, which in resistant to root-knot nematode.

22. Use of paragraph 21, wherein the plant that is resistant is produced by introducing the nucleic acid molecule of any one of the paragraphs 1-3 or 5 optionally together with a nucleic acid molecule of paragraph 4 or 5 into its genome, in particular by means of introgression.

23. Use of any one of the paragraphs 21 or 22, wherein the root-knot nematode is Meloidogyne enterolobii.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A nucleic acid molecule encoding an MeR1 protein, which confers resistance to root-knot nematode when present in a plant of the Solanaceae family, wherein said nucleic acid molecule has a nucleotide sequence which is:
a) a nucleotide sequence comprising a coding sequence according to SEQ ID NO: 2, 3, 4, 5, 6, 7, 8 or 9, or a coding sequence which has at least 90% sequence identity with the sequence according to SEQ ID NO: 2, 3, 4, 5, 6, 7, 8 or 9; or
b) a nucleotide sequence encoding a protein having an amino acid sequence according to SEQ ID NO: 10, 11, 12, 13, 14, 15, 16 or 17, or a protein having an amino acid sequence which has at least 85% sequence identity with the amino acid sequence according to SEQ ID NO: 10, 11, 12, 13, 14, 15, 16 or 17.

2. The nucleic acid of claim 1, wherein the sequence identity with the sequence according to SEQ ID NO: 2, 3, 4, 5, 6, 7, 8 or 9 in order of increased preference is 91%, 92%, 93%, 94%, 95%, 96%, 97%, 98%, 99%.

3. The nucleic acid of claim 1, wherein the sequence identity with the amino acid sequence according to SEQ ID NO: 10, 11, 12, 13, 14, 15, 16 or 17 in order of increased preference is 85%, 86%, 87%, 88%, 89%, 90%, 91%, 92%, 93%, 94%, 95%, 96%, 97%, 98%, 99%.

4. A nucleic acid molecule encoding a NRC6 protein, which contributes to resistance to root-knot nematode when present in a plant of the Solanaceae family together with the nucleic acid molecule of claim 1, wherein said nucleic acid molecule has a nucleotide sequence which is:
a) a nucleotide sequence comprising a coding sequence according to SEQ ID NO: 19, or a coding sequence having at least 98% sequence identity with the sequence according to SEQ ID NO: 19; or
b) a nucleotide sequence encoding a protein having an amino acid sequence according to SEQ ID NO: 20, or a protein having an amino acid sequence which has at least 98% sequence identity with the amino acid sequence according to SEQ ID NO: 20.

5. The nucleic acid molecule of claim 1, which confers resistance to root-knot nematode when present in a Solanum lycopersicum plant.

6. A plant of the Solanaceae family, in particular a Solanum lycopersicum plant, comprising the nucleic acid molecule of claim 1, wherein the plant is resistant to a root-knot nematode.

7. A plant of the Solanaceae family, in particular a Solanum lycopersicum plant, comprising the nucleic acid molecule of claim 1 and at least one nucleic acid molecule of claim 4, wherein the plant is resistant to a root-knot nematode.

8. A plant of the Solanaceae family, in particular a Solanum lycopersicum plant, comprising the nucleic acid molecule of claim 1 and two nucleic acid molecules of claim 4, wherein the plant is resistant to a root-knot nematode.

9. The plant of claim 6, wherein the plant is resistant to Meloidogyne enterolobii.

10. The plant of claim 8, which is a Solanum lycopersicum plant, wherein the nucleic acid molecules are as comprised in the genome of a Solanum lycopersicum plant representative seed of which was deposited with the NCIMB under deposit number NCIMB 43515.

11. The plant of claim 9, which is a Solanum lycopersicum plant, wherein the nucleic acid molecules are as comprised in the genome of a Solanum lycopersicum plant representative seed of which was deposited with the NCIMB under deposit number NCIMB 43515.

12. A rootstock of a plant of the Solanaceae family comprising the nucleic acid molecule of claim 1, and optionally a nucleic acid molecule as claimed in claim 4.

13. A seed, wherein a plant of the Solanaceae family, in particular a Solanum lycopersicum plant, grown from the seed comprises the nucleic acid molecule as defined in claim 1.

14. The seed of claim 13, wherein the Solanum lycopersicum plant grown from the seed is resistant to Meloidogyne enterolobii.

15. A method for selecting a plant of the Solanaceae family carrying the nucleic acid molecule of claim 1, comprising
determining the presence of the nucleic acid molecule of claim 1, or parts thereof, in the plant of the Solanaceae family, and
selecting the Solanaceae plant if it comprises the nucleic acid molecule of claim 1.

16. The method of claim 15, further comprising the step of determining whether the plant shows resistance against a root-knot nematode.

17. The method of claim 15, wherein the plant is a Solanum lycopersicum plant.

18. The method of claim 15, wherein the root-knot nematode is Meloidogyne enterolobii.

19. A method for producing a plant of the Solanaceae family, in particular a Solanum lycopersicum plant, having resistance to root-knot nematode comprising:
(a) crossing a plant comprising the nucleic acid molecule of claim 1, with another plant;
(b) optionally performing one or more rounds of selfing and/or crossing;
(c) selecting after one or more rounds of selfing and/or crossing for a plant that comprises said nucleic acid molecule of claim 1.

20. (canceled)

21. The method of claim 19, wherein the root-knot nematode is Meloidogyne enterolobii.
